(19) United States
(12) Patent Application Publication
(10) Pub. No.: US 2022/0273526 A1
(43) Pub. Date: Sep. 1, 2022

(54) RECOMBINANT SPIDER SILK EXTRUDATE FORMULATIONS

(71) Applicant: Bolt Threads, Inc., Emeryville, CA (US)

(72) Inventors: Lindsay WRAY, Benicia, CA (US); Nour Eldien EL-DIFRAWY, Berkeley, CA (US); Paul Andre GUERETTE, Oakland, CA (US); Maxime BOULET-AUDET, El Cerrito, CA (US); Gregory Wilson RICE, South San Francisco, CA (US); Joshua Tyler KITTLESON, Pleasant Hill, CA (US)

(21) Appl. No.: 17/626,258

(22) PCT Filed: Jul. 11, 2020
(86) PCT No.: PCT/US2020/041735

§ 371 Date: Jan. 11, 2022

Related U.S. Application Data
(60) Provisional application No. 62/975,647, filed on Feb. 12, 2020.
(60) Provisional application No. 62/873,395, filed on Jul. 12, 2019.

(51) Int. Cl. A61K 8/02; A61K 8/04; A61K 8/06; A61K 8/64; A61Q 19/00

(52) U.S. Cl. CPC A61K 8/0204; A61K 8/042; A61K 8/06; A61K 8/64; A61Q 19/00

(57) ABSTRACT

Disclosed herein are recombinant spider silk compositions formed from a silk-based extrudate, such as stable films that adsorb to the skin, and methods for making these compositions. In some embodiments, provided herein is a method of making a silk-based emulsion, comprising: mixing a composition comprising a recombinant spider silk polypeptide powder and glycerol by applying pressure and shear force to the composition, thereby transforming the composition to an extrudate; suspending at least a portion of said extrudate in an aqueous solvent to form an aqueous extrudate suspension; and mixing said aqueous extrudate suspension into an emulsion to form said silk-based emulsion.

### CROSS-REFERENCE TO RELATED APPLICATIONS

This application claims the benefit of U.S. Provisional Application No. 62/873,395, filed Jul. 12, 2019, and U.S. Provisional Application No. 62/975,647, filed Feb. 12, 2020, the contents of which are each incorporated by reference in their entirety

### FIELD OF THE INVENTION

The present disclosure relates to recombinant spider silk compositions formed from a silk-based extrudate, such as stable films that adsorb to the skin.

### BACKGROUND OF THE INVENTION

Silk is a structural protein that has many qualities that make it desirable for use in applications such as skincare and cosmetics. Recent technology has resulted in the scalable production of various recombinant spider silk polypeptides and polypeptides that are derived from recombinant spider silk polypeptides using various host organisms. However, difficulties with solubilizing recovered silk powder in a solution to yield desirable formulations, such as full length silk-based solid or gel compositions has been a significant challenge.

Most cosmetics and skincare product that do incorporate silk try to overcome the solubility problem by using silk that has been hydrolyzed into small amino acid chains. However, these compositions comprising fragments of degraded silk proteins lose the desirable properties of silk. Furthermore, use of harmful solvents is undesirable for use in silk formulations meant to contact the skin.

While new methods of producing sericin-depleted silkworm silk (referred to herein as “silk fibroin”) have resulted in various skincare product that do manage to incorporate full-length (i.e. non-hydrolyzed) silk proteins, the self-aggregation properties of silk can affect the shelf-stability of these products. Specifically, full-length silk fibroin molecules tend to aggregate and precipitate out of solution. Furthermore, these processes are not scalable, and thus are not commercially viable.

Since recombinant spider silk polypeptides form similar secondary and tertiary structures to silk fibroin, it is equally desirable for use in cosmetics and skincare formulations but also can exhibit similar stability and solubility issues due to self-aggregation.

Therefore, what is needed are scalable methods of increasing the solubility and stability of recombinant spider silk polypeptides in silk formulations (e.g., cosmetic and skincare formulations) that do not use harmful solvents and maintain the desirable properties of full length silk proteins.

### SUMMARY

In some embodiments, provided herein is a method of making a silk-based emulsion, comprising: mixing a composition comprising a recombinant spider silk polypeptide powder and glycerol by applying pressure and shear force to the composition, thereby transforming the composition to an extrudate; suspending at least a portion of said extrudate in an aqueous solvent to form an aqueous extrudate suspension; and mixing said aqueous extrudate suspension into an emulsion to form said silk-based emulsion.

In some embodiments, the extrudate is substantially homogenous. In some embodiments, the silk-based emulsion is a cosmetic or skincare formulation.

Also provided herein, in some embodiments, is a method of making a silk-based solid or gel, comprising: mixing a composition comprising a recombinant spider silk polypeptide powder and glycerol by applying pressure and shear force to the composition, thereby transforming the composition to an extrudate; suspending said extrudate in an aqueous solvent to form an aqueous extrudate suspension; and drying said aqueous extrudate suspension to form a silk-based solid or gel. In some embodiments, the method further comprises coagulating said aqueous extrudate suspension to form aggregated silk in said suspension.

In some embodiments, the silk-based solid or gel is a film. In some embodiments, the silk-based solid is a cosmetic or skincare formulation.

Also provided herein, according to some embodiments of the invention, is a method of making a silk-based formulation, comprising: providing a composition comprising a silk protein and a plasticizer; applying pressure and shear force to the composition, thereby transforming the composition to an extrudate; and suspending said extrudate in an aqueous solvent to form an aqueous extrudate suspension.

In some embodiments, the method further comprises drying said aqueous extrudate suspension to form a silk-based solid or gel. In some embodiments, the method further comprises mixing said aqueous extrudate suspension into an emulsion to form said silk-based emulsion. In some embodiments, the method further comprises drying said silk-based emulsion to form a silk-based solid or gel. In some embodiments, the method further comprises adding a coagulant or an additive to said silk-based solid or gel to form a more solid gel or solid. In some embodiments, the method further comprises coagulating said aqueous extrudate suspension to form aggregated silk in said suspension.

In some embodiments, the aqueous extrudate suspension comprises a gel phase, a colloidal phase, and a solution phase. In some embodiments, the method further comprises separating said gel phase, said colloidal phase, or said solution phase from said aqueous extrudate suspension. In some embodiments, the method further comprises drying said gel phase, said colloidal phase, or said solution phase to form a silk-based solid or gel. In some embodiments, the method further comprises separating a mixture of said colloidal phase and said solution phase from said aqueous extrudate suspension. In some embodiments, the method further comprises drying said mixture of said colloidal phase and said solution phase to form a silk-based solid or gel.

In some embodiments, the silk is recombinant spider silk. In some embodiments, the recombinant spider silk comprises full length proteins. In some embodiments, the silk-based solid or gel is a skincare or cosmetic formulation. In some embodiments, the silk-based emulsion is a skincare or cosmetic formulation.

In some embodiments, the plasticizer is glycerin. In some embodiments, the aqueous solution is water. In some embodiments, the coagulant is methanol.

In some embodiments, the extrudate is in a flowable state.

In some embodiments, the silk-based solid or gel is non-toxic. In some embodiments, the silk-based emulsion is non-toxic.

In some embodiments, the applied shear force is at least 1.5 Newton meters. In some embodiments, the applied pressure is at least 1 MPa.

In some embodiments, the method further comprises agitating said aqueous extrudate suspension. In some embodiments, the method further comprises applying heat to said aqueous extrudate suspension.

In some embodiments, the silk-based solid or gel is a film. In some embodiments, the film disperses upon contact with skin or water or gentle rubbing. In some embodiments, the film disperses into a liquid at a temperature of less than 37° C., but more than 23° C.

Also provided herein, according to some embodiments, is a method of making a silk-based gel, colloid or solution, comprising: mixing a composition comprising a silk protein and a plasticizer by applying pressure and shear force to the composition, thereby transforming the composition to an extrudate; suspending said extrudate in an aqueous solvent to form an aqueous suspended extrudate; heating and/or agitating said aqueous suspended extrudate to form a gel phase, a colloidal phase, and solution phase; and separating said phases to generate a silk-based gel, colloid or solution.

In some embodiments, provided herein is a composition comprising: an extrudate comprising a recombinant silk protein and a plasticizer, wherein said extrudate is suspended in an aqueous solution.

In some embodiments, the extrudate suspended in said aqueous solution forms a colloid solution. In some embodiments, the extrudate is is evenly dispersed as particles in said aqueous solution. In some embodiments, the particles in said aqueous solution have a polydispersity index from 0.1 to 0.9. In some embodiments, the particles in said aqueous solution have a z-average of about 600 to 1,000 nm.

In some embodiments, the composition further comprises a coagulant.

In some embodiments, the plasticizer is glycerol.

In some embodiments, the composition is a film. In some embodiments, the film is stable at room temperature and disperses upon contact with skin or water.

In some embodiments, the recombinant silk protein is substantially full length protein. In some embodiments, the recombinant silk protein is not substantially aggregated in said composition. In some embodiments, the recombinant silk protein has a decreased, similar, or increased crystallinity as compared to the recombinant silk protein in powder form.

Also provided herein, according to some embodiments, is a spider silk cosmetic or skincare product comprising an extrudate comprising a silk protein and a plasticizer, wherein said extrudate is dispersed in an aqueous solvent or coagulant in a gel, colloid, or solution phase.

In some embodiments, the extrudate is dispersed in said aqueous solvent and said coagulant. In some embodiments, the said spider silk cosmetic or skincare product is an emulsion or an aqueous solution.

Also provided herein, according to some embodiments, is a spider silk cosmetic or skincare product comprising a solid or semi-solid, wherein said solid or semi-solid comprises dispersed non-aggregated recombinant silk protein and a plasticizer.

In some embodiments, the solid or semi-solid dissolves upon contact with skin. In some embodiments, the solid or semi-solid is a film.

### BRIEF DESCRIPTION OF THE DRAWINGS

The foregoing and other objects, features and advantages will be apparent from the following description of particular embodiments of the invention, as illustrated in the accompanying drawings.

FIG. 1 shows Size Exclusion Chromatography data for P49W21G30 melt compositions extruded under selected heat and RPM conditions, according to various embodiments of the present invention.

FIG. 2 shows Size Exclusion Chromatography data for P65W20G15 melt compositions extruded under selected heat and RPM conditions, according to various embodiments of the present invention

FIG. 3 shows Size Exclusion Chromatography data for P71W19G10 melt compositions extruded under selected heat and RPM conditions, according to various embodiments of the present invention.

FIG. 4 shows a chart of water loss during extrusion for P49W21G30 melt compositions extruded under selected heat and RPM conditions as measured by thermogravimetric analysis (TGA), according to various embodiments of the invention. The data shows % water content of the starting pellet before extrusion and in samples extruded under selected conditions after extrusion.

FIG. 5 shows a chart of water loss during extrusion for P65W20G15 melt compositions extruded under selected heat and RPM conditions as measured by thermogravimetric analysis (TGA), according to various embodiments of the invention. The data shows % water content of the starting pellet before extrusion and in samples extruded under selected conditions after extrusion.

FIG. 6 shows a chart of water loss during extrusion for P71W19G10 melt compositions extruded under selected heat and RPM conditions as measured by thermogravimetric analysis (TGA), according to various embodiments of the invention. The data shows % water content of the starting powder before extrusion and in samples extruded under selected conditions after extrusion.

FIG. 7 shows beta sheet content for P49W21G30 samples extruded under selected heat and RPM conditions as measured by Fourier Transform Infrared Spectroscopy (FTIR). The samples were compared to reference controls of starting protein powder and starting pellets.

FIG. 8 shows beta sheet content for P65W20G15 samples extruded under selected heat and RPM conditions as measured by Fourier Transform Infrared Spectroscopy (FTIR). The samples were compared to reference controls of starting protein powder and starting pellets.

FIG. 9 shows beta sheet content for P71W19G10 samples extruded under selected heat and RPM conditions as measured by Fourier Transform Infrared Spectroscopy (FTIR). The samples were compared to reference controls of starting protein powder and starting pellets.

FIG. 10 shows images of selected extrusion products produced at 20° C. at 10, 100, 200 or 300 RPM captured using polarized light microscopy.

FIG. 11 shows images of selected extrusion products produced at 95° C. at 10, 100, 200 or 300 RPM captured using polarized light microscopy.

FIG. 12 shows a chart of glycerol loss during extrusion for P49W21G30 extrudates extruded under selected heat and RPM conditions as measured by HPLC, according to various embodiments of the invention. The data shows % glycerol content of the starting powder or pellet before extrusion and in samples after extrusion under selected conditions.

FIG. 13 shows a chart of glycerol loss during extrusion for P65W20G15 extrudates extruded under selected heat and RPM conditions as measured by HPLC, according to various embodiments of the invention. The data shows % glycerol content of the starting powder or pellet before extrusion and in samples after extrusion under selected conditions.

FIG. 14 shows a chart of glycerol loss during extrusion for P71W19G10 extrudates extruded under selected heat and RPM conditions as measured by HPLC, according to various embodiments of the invention. The data shows % glycerol content of the starting powder or pellet before extrusion and in samples after extrusion under selected conditions.

FIG. 15 shows a microscope view and macro view (inset) of silk/glycerin extrudate prepared using an Xplore MC15 conical twin screw extruder (Xplore TCE) at 10% silk, 17% silk, or 25% silk in glycerin for the duration and at the temperature shown. An undissolved silk powder in glycerin is shown for reference.

FIG. 16 shows light microscopy images of extrudate circulated in an Xplore TSE extruder at 90° C. for 30 sec, 4 min, 5 min, 10 min, 20 min, 0.5 hours, 1 hour and 1.5 hours.

FIG. 17 shows light microscopy images of extrudate, extrudate resuspended in water at different concentrations, and extrudate resuspended in water after agitation at room temperature or at 90° C.

FIG. 17 shows light microscopy images of extrudate, extrudate resuspended in water at different concentrations, and extrudate resuspended in water after agitation at room temperature or at 90° C.

FIG. 18 shows a macroscopic view and a microscopic view of i) a solution of extrudate suspended in water before phase separation, ii) a gel pellet phase, a colloidal supernatant (i.e., ‘colloidal supe’), and iii) a colloidal phase and a solution phase separated from the colloidal supernatant. Dried film generated from i) the extrudate suspended in water before phase separation, ii) the gel pellet, iii) the colloidal supernatant, and iv) the solution phase is also shown.

FIG. 19 shows a process of making a silk-glycerol emulsion film and applying the film to the skin of a test subject, according to an embodiment of the invention.

FIG. 20 shows a process of making a silk-glycerol emulsion lyophilized film and applying the film to the skin of a test subject, according to an embodiment of the invention.

FIG. 21 shows the process of making and drying i) a suspension of silk-glycerin extrudate and ii) a suspension of silk glycerin slurry (non-extrudate). Also shown are the results of drying each suspension and representative film formation potential of each.

FIG. 22 shows the process of making and drying i) an emulsion comprising silk-glycerin extrudate and ii) an emulsion comprising silk-glycerin slurry (non-extrudate). Also shown are the results of drying each suspension and representative film formation potential of each.

FIG. 23 shows a macroscopic and microscopic view of i) aqueous resuspended extrudate diluted 5× with water and ii) aqueous resuspended extrudate diluted 5× with methanol.

FIG. 24 shows the result of application of a silk-glycerin extrudate dried film i) not exposed to methanol and ii) exposed to methanol to the skin (left). Also shown are the results of the same film compositions rubbed on the skin (right).

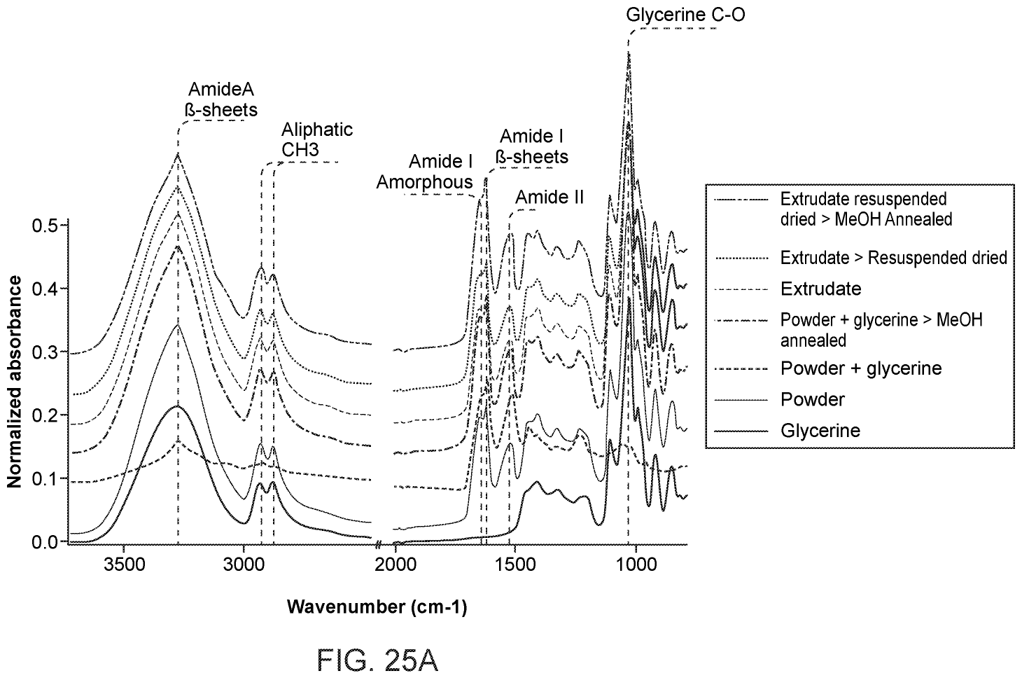

FIG. 25A shows FTIR spectra analyzed for beta-sheet content for selected silk extrudate and non-extrudate compositions described herein. FIG. 25B shows quantitation of relative beta-sheet content of these compositions as determined by FTIR spectra. FIG. 25C shows quantitation of relative amino acid content to glycerin of these compositions as determined by FTIR spectra.

FIG. 26 shows the viscosity of dried down suspensions of 20%, 15%, 10% and 5% extrudate suspended in water and their respective FTIR peaks corresponding to beta sheet content.

FIG. 27 shows a graph of protein concentration (wt %) of aggregate, full-length, and low molecular weight proteins as measured in powder, powder supernatant, extrudate, and extrudate supernatant as measured by size exclusion chromatography.

FIG. 28 shows a size distribution of particles in a extrudate supernatant as measured by a Malvin instrument Zetasizer Nano.

FIG. 29 is an image of a solution of 5% silk powder mixture (left) and 5% silk extrudate supernatant (right) after 24 hours of incubation at 4° C.

FIG. 30 shows a plot of trans epidermal water loss as measured by a vapometer for skin before tape stripping (baseline), after tape stripping (post stripping) and 30 minutes and 2 hours after application of i) no treatment, ii) 15% glycerin in water (vehicle control), and iii) 5% silk protein extrudate mixtures (5% extrudate) to the tape stripped skin.

### DETAILED DESCRIPTION

The details of various embodiments of the invention are set forth in the description below. Other features, objects, and advantages of the invention will be apparent from the description. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

### Definitions

The following terms, unless otherwise indicated, shall be understood to have the following meanings:

The term “stability”, as used herein with respect to silk proteins, refers to the ability of the product not to form a gelation, discoloration or turbidity that is due to the self-aggregation of silk proteins. For example, U.S. Patent Publication No. 2015/0079012 (Wray et al.) is directed to the use of humectant, including glycerol to increase the shelf-stability of skincare products comprising full-length silk fibroin. U.S. Patent Publication No. U.S. Pat. No. 9,187,538 is directed to a skincare formulation comprising full-length silk fibroin that is shelf stable for up to 10 days. Both of these publications are incorporated herein by reference in their entirety.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

The term “glass transition” as used herein refers to the transition of a substance or composition from a hard, rigid or “glassy” state into a more pliable, “rubbery” or “viscous” state.

The term “glass transition temperature” as used herein refers to the temperature at which a substance or composition undergoes a glass transition.

The term “melt transition” as used herein refers to the transition of a substance or composition from a rubbery state to a less-ordered liquid phase or flowable state.

The term “melting temperature” as used herein refers to the temperature range over which a substance undergoes a melt transition.

The term “plasticizer” as used herein refers to any molecule that interacts with a polypeptide sequence to prevent the polypeptide sequence from forming tertiary structures and bonds and/or increases the mobility of the polypeptide sequence.

The term “flowable state” as used herein refers to a composition that has characteristics that are substantially the same as liquid (i.e. has transitioned from a rubbery state into a more liquid state).

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Recombinant Silk Proteins

The present disclosure describes embodiments of the invention including fibers synthesized from synthetic proteinaceous copolymers (i.e., recombinant polypeptides). Suitable proteinaceous co-polymers are discussed in U.S. Patent Publication No. 2016/0222174, published August 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides capable of molded body formation.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is>100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011).

For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent molded bodies that have properties that recapitulate the properties of corresponding molded bodies made from natural silk polypeptides.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of molded body formation.

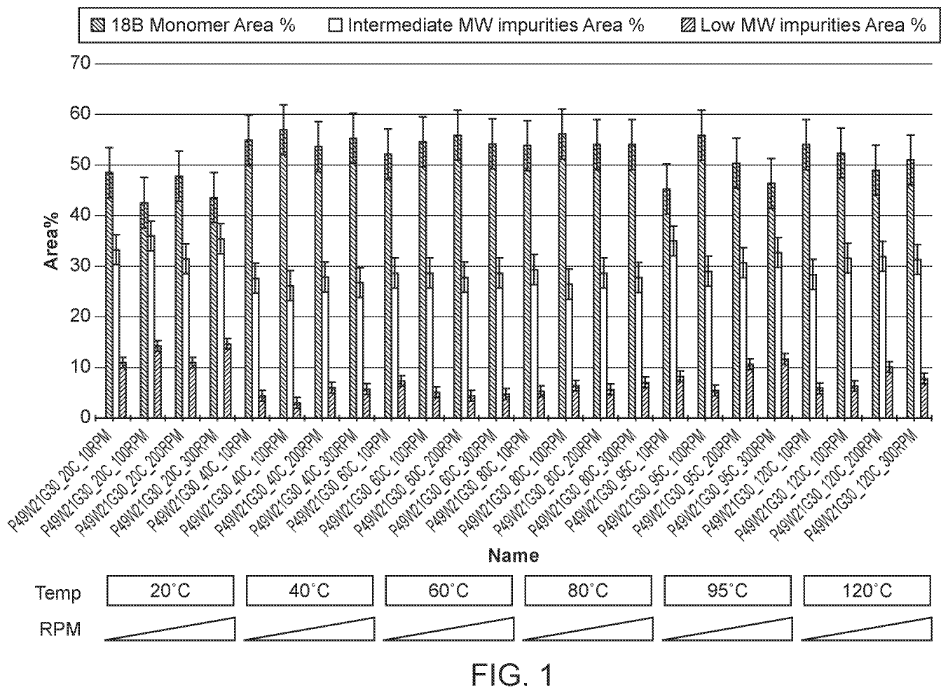

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture, as depicted in FIG. 1. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1A lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 35) is, for the purposes of the present invention, the same as GSGAG (SEQ ID NO: 36) and the same as GGSGA (SEQ ID NO: 37); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1A
Samples of Block Sequences

| Species | Silk Type | Representative Block Amino Acid Sequence |
|---|---|---|
| Aliatypus | Fibroin 1 | GAASSSSTIITTKSASASAAADASAAATASAASRSSANAAASAFAQS |
| gulosus | | FSSILLESGYFCSIFGSSISSSYAAAIASAASRAAAESNGYTTHAYA |
| | | CAKAVASAVERVTSGADAYAYAQAISDALSHALLYTGRLNTANANSL |
| | | ASAFAYAFANAAAQASASSASAGAASASGAASASGAGSAS |
| | | (SEQ ID NO: 8) |
| Plectreurys | Fibroin 1 | GAGAGAGAGAGAGAGAGSGASTSVSTSSSSGSGAGAGAGSGAGSGAG |
| tristis | | AGSGAGAGAGAGGAGAGFGSGLGLGYGVGLSSAQAQAQAQAAAQAQA |
| | | QAQAQAYAAQAQAQAQAQAQAAAAAAAAAAA |
| | | (SEQ ID NO: 9) |
| Plectreurys | Fibroin 4 | GAAQKQPSGESSVATASAAATSVTSGGAPVGKPGVPAPIFYPQGPLQ |
| tristis | | QGPAPGPSNVQPGTSQQGPIGGVGGSNAFSSSFASALSLNRGFTEVI |
| | | SSASATAVASAFQKGLAPYGTAFALSAASAAADAYNSIGSGANAFAY |
| | | AQAFARVLYPLVQQYGLSSSAKASAFASAIASSFSSGTSGQGPSIGQ |
| | | QQPPVTISAASASAGASAAAVGGGQVGQGPYGGQQQSTAASASAAAA |
| | | TATS |
| | | (SEQ ID NO: 10) |
| Araneus | TuSp | GNVGYQLGLKVANSLGLGNAQALASSLSQAVSAVGVGASSNAYANAV |
| gemmoides | | SNAVGQVLAGQGILNAANAGSLASSFASALSSSAASVASQSASQSQA |
| | | ASQSQAAASAFRQAASQSASQSDSRAGSQSSTKTTSTSTSGSQADSR |
| | | SASSSASQASASAFAQQSSASLSSSSSFSSAFSSATSISAV |
| | | (SEQ ID NO: 11) |
| Argiope | TuSp | GSLASSFASALSASAASVASSAAAQAASQSQAAASAFSRAASQSASQ |
| aurantia | | SAARSGAQSISTTTTTSTAGSQAASQSASSAASQASASSFARASSAS |
| | | LAASSSFSSAFSSANSLSALGNVGYQLGFNVANNLGIGNAAGLGNAL |
| | | SQAVSSVGVGASSSTYANAVSNAVGQFLAGQGILNAANA |
| | | (SEQ ID NO: 12) |
| Deinopis | TuSp | GASASAYASAISNAVGPYLYGLGLFNQANAASFASSFASAVSSAVAS |
| spinosa | | ASASAASSAYAQSAAAQAQAASSAFSQAAAQSAAAASAGASAGAGAS |
| | | AGAGAVAGAGAVAGAGAVAGASAAAASQAAASSSASAVASAFAQSAS |
| | | YALASSSAFANAFASATSAGYLGSLAYQLGLTTAYNLGLSNAQAFAS |
| | | TLSQAVTGVGL |
| | | (SEQ ID NO: 13) |
| Nephila | TuSp | GATAASYGNALSTAAAQFFATAGLLNAGNASALASSFARAFSASAES |
| clavipes | | QSFAQSQAFQQASAFQQAASRSASQSAAEAGSTSSSTTTTTSAARSQ |
| | | AASQSASSSYSSAFAQAASSSLATSSALSRAFSSVSSASAASSLAYS |
| | | IGLSAARSLGIADAAGLAGVLARAAGALGQ |
| | | (SEQ ID NO: 14) |
| Argiope | Flag | GGAPGGGPGGAGPGGAGFGPGGGAGFGPGGGAGFGPGGAAGGPGGPG |
| trifasciata | | GPGGPGGAGGYGPGGAGGYGPGGVGPGGAGGYGPGGAGGYGPGGSGP |
| | | GGAGPGGAGGEGPVTVDVDVTVGPEGVGGGPGGAGPGGAGFGPGGGA |
| | | GFGPGGAPGAPGGPGGPGGPGGPGGPGGVGPGGAGGYGPGGAGGVGP |
| | | AGTGGFGPGGAGGFGPGGAGGFGPGGAGGFGPAGAGGYGPGGVGPGG |
| | | AGGFGPGGVGPGGSGPGGAGGEGPVTVDVDVSV |
| | | (SEQ ID NO: 15) |
| Nephila | Flag | GVSYGPGGAGGPYGPGGPYGPGGEGPGGAGGPYGPGGVGPGGSGPGG |
| clavipes | | YGPGGAGPGGYGPGGSGPGGYGPGGSGPGGYGPGGSGPGGYGPGGSG |
| | | PGGYGPGGYGPGGSGPGGSGPGGSGPGGYGPGGTGPGGSGPGGYGPG |
| | | GSGPGGSGPGGYGPGGSGPGGFGPGGSGPGGYGPGGSGPGGAGPGGV |
| | | GPGGFGPGGAGPGGAAPGGAGPGGAGPGGAGPGGAGPGGAGPGGAGP |
| | | GGAGGAGGAGGSGGAGGSGGTTIIEDLDITIDGADGPITISEELPIS |
| | | GAGGSGPGGAGPGGVGPGGSGPGGVGPGGSGPGGVGPGGSGPGGVGP |
| | | GGAGGPYGPGGSGPGGAGGAGGPGGAYGPGGSYGPGGSGGPGGAGGP |
| | | YGPGGEGPGGAGGPYGPGGAGGPYGPGGAGGPYGPGGEGGPYGP |
| | | (SEQ ID NO: 16) |
| Latrodectus | AcSp | GINVDSDIGSVTSLILSGSTLQMTIPAGGDDLSGGYPGGFPAGAQPS |
| hesperus | | GGAPVDFGGPSAGGDVAAKLARSLASTLASSGVFRAAFNSRVSTPVA |
| | | VQLTDALVQKIASNLGLDYATASKLRKASQAVSKVRMGSDTNAYALA |
| | | ISSALAEVLSSSGKVADANINQIAPQLASGIVLGVSTTAPQFGVDLS |
| | | SINVNLDISNVARNMQASIQGGPAPITAEGPDFGAGYPGGAPTDLSG |
| | | LDMGAPSDGSRGGDATAKLLQALVPALLKSDVFRAIYKRGTRKQVVQ |
| | | YVTNSALQQAASSLGLDASTISQLQTKATQALSSVSADSDSTAYAKA |
| | | FGLAIAQVLGTSGQVNDANVNQIGAKLATGILRGSSAVAPRLGIDLS |
| | | (SEQ ID NO: 17) |
| Argiope | AcSp | GAGYTGPSGPSTGPSGYPGPLGGGAPFGQSGFGGSAGPQGGFGATGG |
| trifasciata | | ASAGLISRVANALANTSTLRTVLRTGVSQQIASSVVQRAAQSLASTL |
| | | GVDGNNLARFAVQAVSRLPAGSDTSAYAQAFSSALFNAGVLNASNID |
| | | TLGSRVLSALLNGVSSAAQGLGINVDSGSVQSDISSSSSFLSTSSSS |
| | | ASYSQASASSTS |
| | | (SEQ ID NO: 18) |
| Uloborus | AcSp | GASAADIATAIAASVATSLQSNGVLTASNVSQLSNQLASYVSSGLSS |
| diversus | | TASSLGIQLGASLGAGFGASAGLSASTDISSSVEATSASTLSSSASS |
| | | TSVVSSINAQLVPALAQTAVLNAAFSNINTQNAIRIAELLTQQVGRQ |
| | | YGLSGSDVATASSQIRSALYSVQQGSASSAYVSAIVGPLITALSSRG |
| | | VVNASNSSQIASSLATAILQFTANVAPQFGISIPTSAVQSDLSTISQ |
| | | SLTAISSQTSSSVDSSTSAFGGISGPSGPSPYGPQPSGPTFGPGPSL |
| | | SGLTGFTATFASSFKSTLASSTQFQLIAQSNLDVQTRSSLISKVLIN |
| | | ALSSLGISASVASSIAASSSQSLLSVSA |
| | | (SEQ ID NO: 19) |
| Euprosthenops | MaSp1 | GGQGGQGQGRYGQGAGSSAAAAAAAAAAAAAA |
| australis | | (SEQ ID NO: 20) |
| Tetragnatha | MaSp1 | GGLGGGQGAGQGGQQGAGQGGYGSGLGGAGQGASAAAAAAAA |
| kauaiensis | | (SEQ ID NO: 21) |
| Argiope | MaSp2 | GGYGPGAGQQGPGSQGPGSGGQQGPGGLGPYGPSAAAAAAAA |
| aurantia | | (SEQ ID NO: 22) |
| Deinopis | MaSp2 | GPGGYGGPGQQGPGQGQYGPGTGQQGQGPSGQQGPAGAAAAAAAAA |
| spinosa | | (SEQ ID NO: 23) |
| Nephila | MaSp2 | GPGGYGLGQQGPGQQGPGQQGPAGYGPSGLSGPGGAAAAAAA |
| clavata | | (SEQ ID NO: 24) |
| Deinopis | MiSp | GAGYGAGAGAGGGAGAGTGYGGGAGYGTGSGAGYGAGVGYGAGAGAGG |
| Spinosa | | GAGAGAGGGTGAGAGGGAGAGYGAGTGYGAGAGAGGGAGAGAGAGAGA |
| | | GAGAGSGAGAGYGAGAGYGAGAGAGGVAGAGAAGGAGAAGGAGAAGGA |
| | | GAAGGAGAGAGAGSGAGAGAGGGARAGAGG |
| | | (SEQ ID NO: 25) |
| Latrodectus | MiSp | GGGYGRGQGAGAGVGAGAGAAAGAAAIARAGGYGQGAGGYGQGQGAGA |
| hesperus | | AAGAAAGAGAGGYGQGAGGYGRGQGAGAGAGAGAGARGYGQGAGAGAA |
| | | AGAAASAGAGGYGQGAGGYGQGQGAGAAAGAAASAGAGGYGQGAGGYG |
| | | QGQGA |
| | | (SEQ ID NO: 26) |
| Nephila | MiSp | GAGAGGAGYGRGAGAGAGAAAGAGAGAAAGAGAGAGGYGGQGGYGAGA |
| clavipes | | GAGAAAAAGAGAGGAAGYSRGGRAGAAGAGAGAAAGAGAGAGGYGGQG |
| | | GYGAGAGAGAAAAAGAGSGGAGGYGRGAGAGAAAGAGAAAGAGAGAGG |
| | | YGGQGGYGAGAGAAAAA |
| | | (SEQ ID NO: 27) |
| Nephilengys | MiSp | GAGAGVGGAGGYGSGAGAGAGAGAGAASGAAAGAAAGAGAGGAGGYGT |
| cruentata | | GQGYGAGAGAGAGAGAGGAGGYGRGAGAGAGAGAGGAGGYGAGQGYGA |
| | | GAGAGAAAAAGDGAGAGGAGGYGRGAGAGAGAGAAAGAGAGGAGGYGA |
| | | GQGYGAGAGAGAAAGAGAGGAGGYGAGQGYGAGAGAGAAAAA |
| | | (SEQ ID NO: 28) |
| Uloborus | MiSp | GSGAGAGSGYGAGAGAGAGSGYGAGSSASAGSAINTQTVTSSTTTSSQ |
| diversus | | SSAAATGAGYGTGAGTGASAGAAASGAGAGYGGQAGYGQGAGASARAA |
| | | GSGYGAGAGAAAAAGSGYGAGAGAGAGSGYGAGAAA |
| | | (SEQ ID NO: 29) |
| Uloborus | MiSp | GAGAGYRGQAGYIQGAGASAGAAAAGAGVGYGGQAGYGQGAGASAGAA |
| diversus | | AAAGAGAGRQAGYGQGAGASAGAAAAGAGAGRQAGYGQGAGASAGAAA |
| | | AGADAGYGGQAGYGQGAGASAGAAASGAGAGYGGQAGYGQGAGASAGA |
| | | AAAGAGAGYLGQAGYGQGAGASAGAAAGAGAGYGGQAGYGQGTGAAAS |
| | | AAASSA |
| | | (SEQ ID NO: 30) |
| Araneus | MaSp1 | GGQGGQGGYGGLGSQGAGQGGYGAGQGAAAAAAAAGGAGGAGRGGLGA |
| ventricosus | | GGAGQGYGAGLGGQGGAGQAAAAAAAGGAGGARQGGLGAGGAGQGYGA |
| | | GLGGQGGAGQGGAAAAAAAAGGQGGQGGYGGLGSQGAGQGGYGAGQGG |
| | | AAAAAAAAGGQGGQGGYGGLGSQGAGQGGYGGRQGGAGAAAAAAAA |
| | | (SEQ ID NO: 31) |
| Doloutedes | MaSp1 | GGAGAGQGSYGGQGGYGQGGAGAATATAAAAGGAGSGQGGYGGQGGLG |
| tenebrosus | | GYGQGAGAGAAAAAAAAAGGAGAGQGGYGGQGGQGGYGQGAGAGAAAA |
| | | AAGGAGAGQGGYGGQGGYGQGGGAGAAAAAAAASGGSGSGQGGYGGQG |
| | | GLGGYGQGAGAGAGAAASAAAA |
| | | (SEQ ID NO: 32) |
| Nephilengys | MaSp | GGAGQGGYGGLGGQGAGAAAAAAGGAGQGGYGGQGAGQGAAAAAASGA |
| cruentata | | GQGGYEGPGAGQGAGAAAAAAGGAGQGGYGGLGGQGAGQGAGAAAAAA |
| | | GGAGQGGYGGLGGQGAGQGAGAAAAAAGGAGQGGYGGQGAGQGAAAAA |
| | | AGGAGQGGYGGLGSGQGGYGRQGAGAAAAAAAA |
| | | (SEQ ID NO: 33) |
| Nephilengys | MaSp | GGAGQGGYGGLGGQGAGAAAAAAGGAGQGGYGGQGAGQGAAAAAASGA |
| cruentata | | GQGGYGGPGAGQGAGAAAAAAGGAGQGGYGGLGGQGAGQGAGAAAAAA |
| | | GGAGQGGYGGQGAGQGAAAAAAGGAGQGGYGGLGSGQGGYGGQGAGAA |
| | | AAAGGAGQGGYGGLGGQGAGQGAGAAAAAA |
| | | (SEQ ID NO: 34) |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3× FLAG nucleotide coding sequence. In some embodiments, the silk polypeptide nucleotide coding sequence is operatively linked to other affinity tags such as 6-8 His residues.

In some embodiments, the recombinant spider silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the spider silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, wherein the recombinant spider silk protein comprises repeat units wherein each repeat unit has at least 95% sequence identity to a sequence that comprises from 2 to 20 quasi-repeat units; each quasi-repeat unit comprises {GGY-[GPG-X₁]ₙ₁-GPS-(A)ₙ₂}, (SEQ ID NO: 3) wherein for each quasi-repeat unit; X₁ is independently selected from the group consisting of SGGQQ (SEQ ID NO: 4), GAGQQ (SEQ ID NO: 5), GQGOPY (SEQ ID NO: 6), AGQQ (SEQ ID NO: 7), and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant spider silk polypeptide comprises the polypeptide sequence of SEQ ID NO: 1 (i.e., 18B). In some embodiments, the repeat unit is a polypeptide comprising SEQ ID NO: 2. These sequences are provided in Table 1B:

TABLE 1B
Exemplary polypeptides sequences of recombinant protein and repeat unit

| SEQ ID | Polypeptide Sequence |
|---|---|
| SEQ ID | GGYGPGAGQQGPGSGGQQGPGGQGPYGSGQQGPGGAGQQGPGGQGPYGPGAAAAAAAAAG |
| NO: 1 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAAGGYGPGAGQQGPGSGGQQGPGGQGPYGSGQQGPGGAGQQGPGGQG |
| | PYGPGAAAAAAAAAGGYGPGAGQQGPGGAGQQGPGSQGPGGQGPYGPGAGQQGPGSQGPG |
| | SGGQQGPGGQGPYGPSAAAAAAAAAGGYGPGAGQRSQGPGGQGPYGPGAGQQGPGSQGPG |
| | SGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGP |
| | GSQGPGSGGQQGPGGQGPYGPSAAAAAAAAGGYGPGAGQQGPGSGGQQGPGGQGPYGSGQ |
| | QGPGGAGQQGPGGQGPYGPGAAAAAAAAAGGYGPGAGQQGPGGAGQQGPGSQGPGGQGPY |
| | GPGAGQQGPGSQGPGSGGQQGPGGQGPYGPSAAAAAAAAAGGYGPGAGQRSQGPGGQGPY |
| | GPGAGQQGPGSQGPGSGGQQGPGGQGPYGPSAAAAAAAAGGYGPGAGQQGPGSQGPGSGG |
| | QQGPGGQGPYGPGAAAAAAAVGGYGPGAGQQGPGSQGPGSGGQQGPGGQGPYGPSAAAAA |
| | AAAGGYGPGAGQQGPGSQGPGSGGQQGPGGQGPYGPSAAAAAAAA |
| SEQ ID | GGYGPGAGQQGPGSGGQQGPGGQGPYGSGQQGPGGAGQQGPGGQGPYGPGAAAAAAAAAG |
| NO: 2 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAA |

In some embodiments, the structure of fibers formed from the described recombinant spider silk polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. While not wishing to be bound by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of RPFs. For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities and Degradation

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include, but are not limited to, water and polyalcohols (polyols) such as glycerol, triglycerol, hexaglycerol, and decaglycerol. Other suitable plasticizers include, but are not limited to, Dimethyl Isosorbite; adiptic acid; amide of dimethylaminopropyl amine and caprylic/capric acid; acetamide and any combination thereof.

As hydrophilic portions of silk polypeptides can bind ambient water present in the air as humidity, water will almost always be present, the bound ambient water may plasticize silk polypeptides. In some embodiments, a suitable plasticizer may be glycerol, present either alone or in combination with water or other plasticizers. Other suitable plasticizers are discussed above.

In addition, in instances where recombinant spider silk polypeptides are produced by fermentation and recovered as recombinant spider silk polypeptide powder from the same, there may be impurities present in the recombinant spider silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant spider silk polypeptide powder or composition. Size Exclusion Chromatography separates molecules based on their relative size and can be used to analyze the relative amounts of recombinant spider silk polypeptide in its full-length polymeric and monomeric forms as well as the amount of high, low and intermediate molecular weight impurities in the recombinant spider silk polypeptide powder. Similarly, Rapid High Performance Liquid Chromatography may be used to measure various compounds present in a solution such as monomeric forms of the recombinant spider silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant spider silk polypeptide may have a purity calculated based on the amount of the recombinant spider silk polypeptide in is monomeric form by weight relative to the other components of the recombinant spider silk polypeptide powder. In various instances, the purity can range from 50% by weight to 90% by weight, depending on the type of recombinant spider silk polypeptide and the techniques used to recover, separate and post-process the recombinant spider silk polypeptide powder.

Both Size Exclusion Chromatography and Reverse Phase High Performance Liquid Chromatography are useful in measuring full-length recombinant spider silk polypeptide, which makes them useful techniques for determining whether processing steps have degraded the recombinant spider silk polypeptide by comparing the amount of full-length spider silk polypeptide in a composition before and after processing. In various embodiments of the present invention, the amount of full-length recombinant spider silk polypeptide present in a composition before and after processing may be subject to minimal degradation. The amount of degradation may be in the range 0.001% by weight to 10% by weight, or 0.01% by weight to 6% by weight, e.g. less than 10% or 8% or 6% by weight, or less than 5% by weight, less than 3% by weight or less than 1% by weight.

### Measuring Glass Transition Temperature (Tg), Secondary and Tertiary Structures

In some embodiments, Differential Scanning calorimetry is used to determine the glass transition and/or melt transition temperature of the recombinant spider silk polypeptide and/or fiber containing the same. In a specific embodiment, Modulated Differential Scanning calorimetry is used to measure the glass transition and/or melt transition temperature.

Depending on the embodiment and the type of recombinant spider silk polypeptide, the glass transition and/or melt transition temperatures may have range of values. However, a measured glass transition and/or melt transition temperature that is much lower than is typically observed for a recombinant spider silk polypeptide in its solid form may indicate that impurities or the presence of other plasticizers.

In addition, Fourier Transform Infrared (FTIR) spectroscopy data may be combined with rheology data to provide both direct characterization of tertiary structures in the recombinant silk powder and/or composition containing the same. FTIR can be used to quantify secondary structures in silk polypeptides and/or composition comprising the silk polypeptides as discussed below in the section entitled “Fourier Transform Infrared (FTIR) Spectroscopy.”

Depending on the embodiment, FTIR may be used to quantify beta-sheet structures present in the recombinant spider silk polypeptide powder and/or composition containing the same. In addition, in some embodiments, FTIR may be used to quantify impurities such as sugars and lipids present in the recombinant spider silk polypeptide powder. However, various chaotropes and solubilizers used in different protein pre-processing methods may diminish the number of tertiary structures in recombinant spider silk polypeptide powder or composition containing the same. Accordingly, there may be no correspondence between the amount of beta sheet structures in recombinant spider silk polypeptide powder before and after it is molded or spun into fiber. Similarly, there may be little to no correspondence between the glass transition temperature of a powder before and after it is molded or spun into fiber.

Fourier Transform Infrared (FTIR) spectra can be used to assess the tertiary structure of proteins present in polypeptide powder and/or fibers. Specifically, FTIR spectra can be used to determine the amount of beta sheets present in the fibers that are subject to different spinning and post-processing conditions. Thus, FTIR spectra may be used to determine the relative amount of beta sheet structures based on the different techniques. Alternately, the FTIR spectra may be compared to native insect silk.

Depending on the embodiment, FTIR spectra at different wavenumbers may be used to assess the different tertiary structures present in the fibers. In various embodiments, wavenumbers corresponding to Amide I and Amide II bands may be used to assess various protein structures such as turns, beta-sheets, alpha helices, and side chains. Wavenumbers corresponding to these structures are well known in the art.

In most embodiments, FTIR spectra at wavenumbers corresponding to beta sheets will be used to assess the quantity of beta sheet structures in the polypeptide powder and/or fiber. In a specific embodiment, FTIR spectra at 982-949 cm⁻¹(CH₂ rocking (A)ₙ), 1695-1690 cm⁻¹ (Amide I) 1620-1625 cm⁻¹ (Amide I), 1440-1445 cm⁻¹ (asymmetric CH₃ bending) and/or 1508 cm⁻¹ (Amide II) are used to determine the amount of beta sheets present. Depending on the embodiment, the different wavenumbers and ranges can be measured to determine the amount of beta sheets present. In some embodiments the FTIR spectra at 982-949 cm⁻¹ is used in order to eliminate interference from corresponding peaks. Exemplary methods of obtaining spectra at these wavenumbers are discussed in detail in Boudet-Audet et al, Identification and classification of silks using infrared spectroscopy, Journal of Experimental Biology, 218:3138-3149 (2015), the entirety of which is herein incorporated by reference.

Similarly, various methods of characterizing impurities in the recombinant silk powder may be combined with rheological and/or FTIR data to analyze the relationship between the presence of impurities and the formation of secondary and/or tertiary structures.

### Recombinant Spider Silk Melt Compositions

It is an object of this invention to make various recombinant spider silk compositions that are capable of being transformed into a melted or flowable state (i.e., capable of being transformed into a recombinant spider silk melt composition) according to the methods described herein. In various embodiments, the concentration of recombinant spider silk polypeptide powder and plasticizer in the composition may be varied based on the properties of the recombinant spider silk polypeptide powder (e.g., the purity of the recombinant spider silk polypeptide powder), the type of plasticizer used, and the desired properties of the fiber. In some embodiments, concentrations may be adjusted based on rheological data such as the data from a Capillary Rheometer.

Depending on the embodiment, suitable concentrations of recombinant spider silk polypeptide powder by weight in the recombinant spider silk composition ranges from: 1 to 25% by weight, 1 to 30% by weight, to 70% by weight, 10 to 60% by weight, 15 to 50% by weight, 18 to 45% by weight, or 20 to 41% by weight.

In the instance where glycerol is used as a plasticizer, suitable concentration of glycerol by weight in the recombinant spider silk composition ranges from: 1 to 90% by weight, 10 to 90% by weight, 10 to 50% by weight, 10 to 40% by weight, 15 to 40% by weight, 10 to 30% by weight, or 15 to 30% by weight.

In the instance where water is used as a plasticizer, a suitable concentration of water by weight in the recombinant spider silk composition ranges from: 5 to 80% by weight, 15 to 70% by weight, 20 to 60% by weight, 25 to 50% by weight, 19 to 43% by weight, or 19 to 27% by weight. Where water is used in combination with another plasticizer, it may be present in the range 5 to 50% by weight, 15 to 43% by weight or 19 to 27% by weight.

In some embodiments, water may be evaporated during extrusion and/or cooling process depending the treatment and/or the die size used. In some embodiments, water loss after molding may range from 1 to 50% by weight, 3 to 40% weight, 5 to 30% weight, 7 to 20% weight, 8 to 18% weight, or 10-15% based on the total water amount. Often loss will be less than 15%, in some cases less than 10%, for instance 1 to 10% by weight. Evaporation may be intentional or as a result of the treatment applied. The degree of evaporation can be easily controlled, for instance by selection of operating temperatures, flow rates and pressures applied, as would be understood in the art.

In some embodiments, suitable plasticizers may include polyols (e.g., glycerol), water, lactic acid, ascorbic acid, phosphoric acid, ethylene glycol, propylene glycol, triethanolamine, acid acetate, propane-1,3-diol or any combination thereof.

In various embodiments, the amount of plasticizer can vary according to the purity and relative composition of the recombinant spider silk polypeptide powder. For example, a higher purity powder may have less impurities such as a low molecular weight compounds that may act as plasticizers and therefore require the addition of a higher percentage by weight of plasticizer.

Without intending to be limited by theory, in various embodiments of the present invention, inducing the recombinant spider silk composition to transition into a flowable state (e.g. inducing a recombinant spider silk melt composition) may be used as a pre-processing step in any formulation in circumstances where it is beneficial to include the recombinant spider silk polypeptide in its monomeric form. More specifically, inducing the recombinant spider silk melt composition may be used in applications where it is desirable to prevent the aggregation of the monomeric recombinant spider silk polypeptide into its crystalline polymeric form or to control the transition of the recombinant spider silk polypeptide into its crystalline polymeric form at a later stage in processing.

According some embodiments of the present invention, the recombinant spider silk composition is transformed into melted or flowable state through the application of shear force and/or pressure, typically both. Suitable means for generating a combination of shear force and pressure include but are not limited to: single screw extruders, twin screw extruders, melt flow extruders, and capillary rheometers.

In some embodiments, a twin screw extruder is used to provide the necessary pressure and shear force to transform the recombinant spider silk composition into a melted or flowable composition. In some embodiments, the twin screw extruder is configured to provide a shear force ranging from: 1.5 Newton meters (Nm) to 13 Newton meters, 2 Newton meters to 10 Newton meters, 2 Newton meters to 8 Newton meters, or 2 Newton meters to 6 Newton meters. In some embodiments, the shear force provided by the twin screw extruder depends, in part, on the rotations per minute of the twin screw extruder. In various embodiments and configurations the rotations per minute (RPMs) of the twin screw extruder may range from 10 RPMs to 1,000 RPMs. In various embodiments, the twin screw extruder is configured to provide a pressure ranging from 1 MPa to 300 MPa in conjunction with the shear force.

In optional embodiments, the twin screw extruder is configured to apply heat to the recombinant spider silk composition before and/or after it is transformed into a recombinant spider silk melt composition. In some embodiments, the barrel of the twin screw extruder (i.e. the cylinder in which the twin screws mix a composition) is subject to heating. In other embodiments, a portion of the twin screw extruder proximal to a spinneret (i.e. orifice through which the recombinant spider silk melt composition is extruded) is subject to heating. Alternatively, no heat is applied, the melt/flowable state being induced entirely through heat generated from the shearing forced applied to the recombinant spider silk composition in the twin screw extruder. For example, in some embodiments, the amount of heat applied to obtain a melt/flowable state would be similar to equal to ambient room temperature (e.g. approximately than 20° C.).

In various embodiments, the temperature to which the recombinant spider silk melt composition is heated will be minimized in order to minimize or entirely prevent degradation of the recombinant spider silk polypeptide. In specific embodiments, the recombinant spider silk melt will be heated to a temperature of less than 120° C., less than 100° C., less than 80° C., less than 60° C., less than 40° C., or less than 20° C. Often the melt will be at a temperature in the range 10° C. to 120° C., 10° C. to 100° C., 15° C. to 80° C., 15° C. to 60° C., 18° C. to 40° C. or 20±2° C. during processing.

In other embodiments, other devices may be used to provide pressure and shear force necessary to transform the recombinant spider silk composition into a melted or flowable state. As discussed above, a capillary rheometer may also be used to provide the necessary shear force and pressure to transform the recombinant spider silk composition into a flowable or melted state.

In some embodiments, the recombinant spider silk composition is optionally heated after it is in a melted or flowable state and/or prior to extrusion of the melted or flowable recombinant spider silk melt composition. Where heating is required, perhaps because the recombinant spider silk composition is of high glass transition temperature, the device used to provide shear force and pressure to transform the recombinant spider silk composition into a melted or flowable state may be coupled, either directly or indirectly to a heated extrusion device. In a specific embodiment, a twin screw cylinder mixer is coupled (either directly or indirectly) to a heated extrusion device. Depending on the embodiment and configuration of the heated extrusion device, the heated extrusion device may be maintained at temperatures ranging from 20 to 120° C., 80 to 110° C., 85 to 100° C., 85 to 95° C. and/or 90 to 95° C.

The extruded recombinant spider silk melt composition is herein referred to as a “recombinant spider silk extrudate.” Depending on the application of the recombinant spider silk extrudate, the spinneret through which the extrudate is extruded may vary in diameter. For example, in embodiments where the recombinant spider silk extrudate is extruded into a mold to form a molded object, the spinneret may have a diameter greater than 200 mm, greater than 150 mm, greater than 100 mm, greater than 50 mm for instance in the range 100 mm to 500 mm, 150 mm to 400 mm or 200 mm to 300 mm. As discussed below, in some embodiments the recombinant spider silk extrudate can be processed into pellets that may be re-processed by again subjecting the pellets to shear force and pressure sufficient to transform the spider silk extrudate into a recombinant spider silk melt composition. In embodiments where the recombinant spider silk extrudate is processed into pellets, the spinneret may have a diameter greater than 2 mm, greater than 1.5 mm or greater than 1 mm, for instance, the diameter may be in the range 1 mm to 5 mm, 1.5 mm to 4 mm, or 2 mm to 3 mm.

In most embodiments of the present invention, both the recombinant spider silk melt composition and the recombinant spider silk extrudate will be substantially homogeneous meaning that the material, as inspected, e.g., by light microscopy or UV/VIS of resuspended extrudate, does not have any inclusions or precipitates. In some embodiments, light microscopy may be used to measure birefringence which can be used as a proxy for alignment of the recombinant spider silk into a three-dimensional lattice. Birefringence is the optical property of a material having a refractive index that depends on the polarization and propagation of light. Specifically, a high degree of axial order as measured by birefringence can be linked to high tensile strength. In some embodiments, recombinant spider silk melt extrudate will have minimal birefringence.

According to the present invention, a homogeneous flowable state can be induced through the application of shear force and pressure only, although optionally heat may be applied. The combination of shear force and pressure alone, without the application of heat or with optional heat, has been found to provide compositions which do not degrade during processing of the recombinant spider silk polypeptide in the recombinant spider silk melt composition and the recombinant spider silk extrudate. This is desirable and beneficial as retaining the full length recombinant spider silk polypeptide in the extrudate composition produces optimal material properties, such as crystallinity, resulting in higher quality products. In embodiments of the present invention, the recombinant spider silk melt extrudate achieved from the application of shear force and pressure (and optionally heat) has minimal or negligible degradation.

The amount of degradation of the recombinant spider silk polypeptide may be measured using various techniques. As discussed above, the amount of degradation of the recombinant spider silk polypeptide may be measured using Size Exclusion Chromatography to measure the amount of full-length recombinant spider silk polypeptide present. In various embodiments, the composition is degraded in an amount of less than 6.0 weight % after it is formed into a molded body. In another embodiment, the composition is degraded in an amount of less than 4.0 weight % after molding, less than 3.0 weight %, less than 2.0 weight %, or less than 1.0 weight % (such that the amount of degradation may be in the range 0.001% by weight to 10%, 8%, 6%, 4%, 3%, 2% or 1% by weight, or 0.01% by weight to 6%, 4%, 3%, 2% or 1% by weight). In another embodiment, the recombinant spider silk protein in the extrudate and/or melt composition is substantially non-degraded.

Compounding Extrudate into Cosmetics Formulations

In various embodiments, the recombinant spider silk extrudate will be compounded into a spider silk cosmetic or skincare product (e.g., solutions applied to the skin or hair). Specifically, the recombinant spider silk extrudate may be used as a base for a cosmetic or skincare product where the recombinant spider silk polypeptide is present in the base in its monomeric or less-crystalline form. Without intending to be limited by theory, subjecting the recombinant spider silk polypeptide to shear force and pressure in the presence of a plasticizer such as glycerol coverts the recombinant spider silk polypeptide into an “open-form recombinant spider silk polypeptide” in which the recombinant spider silk polypeptide unfolds and forms interactions with the glycerol. Due to the interactions with glycerol, this “open-form recombinant spider silk polypeptide” forms less intermolecular and intramolecular beta-sheet interactions. Specifically, the open form recombinant spider silk polypeptide is prevented from forming intermolecular interactions to form an irreversible three-dimensional lattice.

Without intending to be limited by theory, compounding the open-form recombinant spider silk polypeptide in a skincare formulation allows for the controlled aggregation of the recombinant spider silk polypeptide into its crystalline polymeric form upon contact with skin or through various other chemical reactions. Similarly, maintaining the open-form recombinant spider silk polypeptide in its less-crystalline form may increase stability of the recombinant spider silk polypeptide in the cosmetic or skincare product by preventing self-aggregation of the recombinant spider silk polypeptide. As described below, in various embodiments, the recombinant spider silk extrudate may form a semi-solid or gel-like structure that is dispersible at a relatively low melting temperature (Tm). In various embodiments where the recombinant spider silk extrudate is compounded into a skin care formulation, the recombinant spider silk extrudate may form a reversible three-dimensional structure such as a gel or film that melts into a dispersible liquid upon the surface of the skin.

In various embodiments, the recombinant spider silk extrudate may be suspended in water (“aqueous suspended extrudate”) to form a gel or base that can be incorporated (i.e. compounded) in a cosmetic or skincare formulation. Depending on the embodiment, the amount of recombinant spider silk extrudate to water in the aqueous suspended extrudate can vary, as can the relative ratio of recombinant spider silk polypeptide powder to glycerol in the recombinant spider silk extrudate. In some embodiments, the extrudate composition will comprise 10-33% recombinant silk polypeptide powder by weight and 67-90% glycerol by weight. In some embodiments, a different plasticizer than glycerol will be used. In some embodiments, the recombinant spider silk extrudate is suspended in water to create an aqueous suspended extrudate that is 1-40% recombinant spider silk extrudate and 60-99% water. In a specific embodiment, the extrudate composition is suspended in water to create an aqueous suspended extrudate that is 10% recombinant silk polypeptide powder by weight, 30% glycerol by weight and 60% water by weight. In a specific embodiment, the extrudate is suspended in water to create an aqueous suspended extrudate that is 6% recombinant silk polypeptide powder by weight, 18% glycerol by weight and 76% water by weight.

Depending on the embodiment, the aqueous suspended extrudate may be optionally heated and agitated when it is re-suspended in water. In some embodiments, heating and agitating the aqueous suspended extrudate may result in a phase transformation of the recombinant spider silk polypeptides in the aqueous suspended extrudate. Specifically, heating and agitating the aqueous suspended extrudate results in three distinct phases that are assessed by centrifugation: 1) a gel phase that is distinct from the supernatant after centrifugation; 2) a colloidal phase that can be filtered from the supernatant after centrifugation; and 3) a solution phase that remains after filtering the colloidal phase from the supernatant. Various combinations of heat, agitation and centrifugation may be used, provided that the aqueous suspended extrudate must not be subject to prolonged heat in order to prevent degradation of the recombinant spider silk polypeptides. In a specific embodiment, the extrudate is subjected to gentle agitation at 90° C. for 5 minutes and centrifuged at 16,000 RCF for 30 minutes.

In some embodiments, either the various phases of the aqueous suspended extrudate (i.e. colloidal phase, gel phase and solution) or the aqueous suspended extrudate may be incorporated in a cosmetic or skincare formulation to provide a source of open-form recombinant spider silk protein. Depending on the embodiment, the aqueous suspended extrudate may subject to agitation with or without heat before incorporating into a skincare formulation. Optionally, the aqueous suspended extrudate may be separated in the above-discussed phases by centrifugation and/or filtering. Depending on the embodiment, the skincare formulation may be an emulsion (e.g. a cream or serum) or a primarily aqueous solution (e.g. a gel). In certain embodiments, the recombinant spider silk extrudate may be incorporated into any of the above-discussed cosmetic or skincare formulation without aqueous resuspension. In these compositions, a homogenizer or similar equipment may be used to ensure that the recombinant spider silk extrudate is uniformly distributed in the composition.

In some embodiments, the colloid phase (i.e., colloid suspension) comprises particles of various sizes comprising recombinant spider silk protein. In some embodiments, the particle sizes range in diameter from 1 nm to 10,000 nm, from 10 nm to 5,000 nm, or from 20 nm to 3000 nm. In some embodiments, the majority of particles in the colloid suspension range from 50 nm to 2,000 nm. In some embodiments, the colloid suspension has an average particle diameter of about 350 nm. In some embodiments, the average particle diameter is from 300 nm to 400 nm, from 200 nm to 500 nm, or from 100 nm to 1,000 nm. In some embodiments, the colloid suspension has a polydispersity index as measured by a Malvern instrument Zetasizer Nano of about 0.5. In some embodiments, the polydispersity index is from 0.4 to 0.6, from 0.3 to 0.7, from 0.2 to 0.8, or from 0.1 to 1.0. In some embodiments, the polydispersity index is greater than 0.05, greater than 0.1, greater than 0.2, greater than 0.3, or greater than 0.4. In some embodiments, the distribution of particles in the colloid suspension comprises two or more peaks.

In some embodiments, the aqueous suspended extrudate may be subject to heat and agitation, then cast onto a flat surface and dried into a film. In some embodiments, the aqueous suspended extrudate may be incorporated into an emulsion, then cast onto a flat surface and dried into a film. Depending on the embodiment, various different drying conditions may be used. Suitable drying conditions include drying at 60° C. with and without a vacuum. In embodiments that use a vacuum, 15 Hg is a suitable amount of vacuum. Other methods of drying are well established in the art.

In various embodiments, the films comprising the aqueous suspended extrudate alone and in an emulsion have a low melting temperature. In various embodiments, the films comprising the aqueous suspended extrudate alone and in an emulsion have melting temperature that is less than body temperature (around 34-36 C) and melts upon contract with skin. Without intending to be limited by theory, the open form recombinant spider silk polypeptide forms enough intermolecular interactions to make a semi-solid structure (i.e. film), however this structure is reversible upon skin contact and can be re-formed after dispersion on the skin surface. As discussed below, a slurry of recombinant silk polypeptide powder and glycerol and then suspended in an aqueous solution, does not form a film upon drying but forms the same slurry as before suspension. In various embodiments, the film will have reduced crystallinity compared to the recombinant spider silk polypeptide powder or the recombinant spider silk extrudate, as measured by FTIR.

In another specific embodiment, the aqueous suspended extrudate or the extrudate may be incorporated (e.g., homogenized) into an emulsion, then cast on a flat surface and lyophilized to create a porous film. Depending on the embodiment, various techniques may be used for lyophilization, including freezing the film at −80° C. for 30 minutes. Other lyophilization techniques will be well known to those skilled in the art. In various embodiments, the lyophilized porous films comprising the aqueous suspended extrudate alone and in an emulsion have melting temperature that is less than body temperature (around 34-36° C.) and melts upon contract with skin.

In various embodiments, the above-described films that can be used as a topical skincare agent. This film may be applied directly to the skin and can be re-hydrated to form a dispersible viscous substance that is incorporated into the skin. As discussed below, various emollients, humectants, active agents and other cosmetic adjuvants may be incorporated into the film. This film may be applied directly to the skin and adsorb to the skin due to contact with the skin, or after gently rubbing the mask into the skin. In some embodiments, the extrudate resuspended in an aqueous solution may be applied to the face and then exposed to a coagulant such as propylene glycol via mist to form a gellable mask.

Depending on the embodiment, the film that is cast may be a flat film (i.e. with no surface variability) may be cast on a mold that incorporates microstructures. In a specific embodiment, the film that is cast on a mold that incorporates microneedle structures to prick the surface of the skin and assist in delivery of active agents.

In an alternate embodiment, the aqueous suspended extrudate may be added to an emulsion that is used as a skin care product. The emulsion may be applied to skin and then allowed to form a film on the surface of the skin upon drying. As discussed below, various emollients, humectants, active agents and other cosmetic adjuvants may be incorporated into the emulsion.

### Compositions Comprising Emulsions and Films

The emulsions and films discussed above may contain various humectants, emollients, occlusive agents, active agents and cosmetic adjuvants, depending on the embodiment and the desire efficacy of the formulation.

The term “humectant” as used herein refers to a hygroscopic substance that forms a bond with water molecules. Suitable humectants include but are not limited to glycerol, propylene glycol, polyethylene glycol, pentalyene glycol, tremella extract, sorbitol, dicyanamide, sodium lactate, hyaluronic acid, aloe vera extract, alpha-hydroxy acid and pyrrolidonecarboxylate (NaPCA). The term “emollient” as used herein refers to a compound that provide skin a soft or supple appearance by filling in cracks in the skin surface. Suitable emollients include but are not limited to shea butter, cocao butter, squalene, squalane, octyl octanoate, sesame oil, grape seed oil, natural oils containing oleic acid (e.g. sweet almond oil, argan oil, olive oil, avocado oil), natural oils containing gamma linoleic acid (e.g. evening primrose oil, borage oil), natural oils containing linoleic acid (e.g. safflower oil, sunflower oil), or any combination thereof. The term “occlusive agent” refers to a compound that forms a barrier on the skin surface to retain moisture. In some instances, emollients or humectants may be occlusive agents. Other suitable occlusive agents may include but are not limited to beeswax, canuba wax, ceramides, vegetable waxes, lecithin, allantoin. Without being limited to theory, the film-forming capabilities of the recombinant spider silk compositions presented herein make an occlusive agent that forms a moisture retaining barrier because the recombinant spider silk polypeptides act attract water molecules and also act as humectants.

In some embodiments, the emulsions and films described herein form a barrier on the skin surface that prevents reduces trans epidermal water loss from damaged skin. In some embodiments, the trans epidermal loss as measured by a vapometer is less than 10 after application of a said barrier on the skin surface. In some embodiments, the trans epidermal water loss is reduced by more than 25%, more than 30%, more than 35%, more than 40%, more than 45%, more than 50%, more than 55%, more than 60%, more than 65%, more than 70%, or more than 75% as compared to untreated damaged skin.

The term “active agent” refers to any compound that has a known beneficial effect in skincare formulation or sunscreen. Various active agents may include but are not limited to acetic acid (i.e. vitamin C), alpha hydroxyl acids, beta hydroxyl acids, zinc oxide, titanium dioxide, retinol, niacinamide, other recombinant proteins (either as full length sequences or hydrolyzed into subsequences or “peptides”), copper peptides, curcuminoids, glycolic acid, hydroquinone, kojic acid, l-ascorbic acid, alpha lipoic acid, azelaic acid, lactic acid, ferulic acid, mandelic acid, dimethylaminoethanol (DMAE), resveratrol, natural extracts containing antioxidants (e.g. green tea extract, pine tree extract), caffeine, alpha arbutin, coenzyme Q-10, and salicylic acid. The term “cosmetic adjuvant” refers to various other agents used to create a cosmetic product with commercially desirable properties including without limitation surfactants, emulsifiers, preserving agents and thickeners.

### Coagulants

In some embodiments, a silk-based composition produced herein is exposed to a coagulant. This can change the properties of the composition to facilitate controlled aggregation of silk in the silk-based composition. In some embodiments, the silk-based composition is submerged in a coagulant. In some embodiments, the silk-based composition is exposed to a coagulant mist or vapor. In one embodiment, an aqueous extrudate composition comprises or is submerged with or mixed with a coagulant. In some embodiments, a silk-based solid or semi-solid, such as a film is submerged in or exposed to a vapor comprising coagulant. In some embodiments, methanol is used as an effective coagulant.

In some embodiments, alcohol can be used as a coagulant, such as isopropanol, ethanol, or methanol. In some embodiments, 60%, 70%, 80%, 90% or 100% alcohol is used as a coagulant. In some embodiments, a salt can be used as a coagulant, such as ammonium sulfate, sodium chloride, sodium sulfate, or other protein precipitating salts effective at a temperature from 20 to 60° C.

In some embodiments, a combination of one or more of water, acids, solvents and salts, including but not limited to the following classes of chemicals of Brönsted-Lowry acids, Lewis acids, binary hydride acids, organic acids, metal cation acids, organic solvents, inorganic solvents, alkali metal salts, and alkaline earth metal salts can be used as a coagulant. In some embodiments, the acids comprise dilute hydrochloric acid, dilute sulfuric acid, formic acid or acetic acid. In some embodiments the solvents comprise ethanol, methanol, isopropanol, t-butyl alcohol, ethyl acetate, propylene glycol, or ethylene glycol. In some embodiments, the salts comprise LiCl, KCl, BeCl₂, MgCl₂, CaCl₂, NaCl, ZnCl₂, FeCl₃, ammonium sulfate, sodium sulfate, sodium acetate, and other salts of nitrates, sulfates or phosphates. In some embodiments, the coagulant is at a pH from 2.5 to 7.5.

### EXAMPLES

### Example 1: Purity of Recombinant 18B Polypeptide Powder

Recombinant spider silk—18B polypeptide sequences (SEQ ID NO: 1) comprising the FLAG tag—were produced through various lots of large-scale fermentation, recovered and dried in powders (“18B powder”). Reverse Phase High Performance Liquid Chromatography (“RP-HPLC”) was used to measure the amount by weight of 18B polypeptide monomer in the powder. The samples were dissolved using a 5M Guanidine Thiocyanate (GdSCN) reagent and injected onto an Agilent Poroshell 300SB C3 2.1×75 mm 5 μm column to separate constituents on the basis of hydrophobicity. The detection modality was UV absorbance of peptide bond at 215 nm (360 nm reference). The sample concentration of 18B-FLAG monomer was determined by comparison with an 18B-FLAG powder standard, for which the 18B-FLAG monomer concentration had been previously determined using Size Exclusion Chromatography (SEC-HPLC)

The sample powder was found to include 57.964 Mass % of 18B monomer.

### Example 2: Silk Powder Extrudate Mixtures

Silk extrudate mixtures were formed as follows: The recombinant silk powder of Example 1 was mixed using a household spice grinder. Ratios of water and glycerol were added to the recombinant silk powder (“18B powder”) to generate recombinant spider silk compositions with different ratios of protein powder to plasticizer as tabulated below in Table 2.

Batches of 10 to 100 grams of the recombinant spider silk compositions (i.e., “formulations”) listed below in Table 2 were mixed using a Xceptional Instruments Twin Screw Extruder (TSE) (item number TT-ZE5-MSMS-3HT) which was used for all TSE experiments. The stainless steel (S316) extruder barrel had 3 heating zones ˜5 cm in length each. The screws used were a standard pair of stainless steel (S316) co-rotating screws 180 mm in length and 9 mm in diameter and (L/D ratio of 20:1). The screws had a pitch of 9 mm.

For the P49W21G30 and P65W20G15 formulations listed below, recombinant spider silk compositions were first extruded into pellets that were re-processed in the following experiments by re-extruding the pellets. To make pellets, recombinant spider silk compositions comprising 18B/Water/Glycerol mixtures were introduced to the TSE using a metallic funnel and pushed into contact with the twin screws using a tamping device continuously for several minutes while the TSE was running at 300 RPM with a temperature of ˜90-95° C. across all three barrel regions including the start, middle and end barrel regions. The material was extruded in the melt state (i.e., as a recombinant spider silk melt composition) through a 0.5 mm die whose orifice was at a 180° angle to the screw axis to form a recombinant spider silk extrudate.

The 0.5 mm recombinant spider silk extrudates emerged from the die as continuous, elastomeric “noodles” ˜>10 meters in length. Pellets were generated by sequentially placing 5-10 g quantities of corresponding extrudates compositions into a kitchen spice grinder and subjecting them to 5 second pulses for a total of 6 pulses (30 seconds total). The pellets were inspected to ensure they had lengths of no more than 5 mm, with average lengths of pellets being about 2.5 mm.

For the P71W19G10 formulation listed below, the 18B/water/glycerol recombinant spider silk mixture was pre-mixed and extruded directly (i.e. without first extruding as a pellet) under the conditions described in Example 2 to form recombinant spider silk extrudate.

TABLE 2
Recombinant Spider Silk Formulations Composition by Weight

| | 18 B | Water | Glycerol |
|---|---|---|---|
| | Powder % | % by | % |
| Formulation | by weight | weight | by weight |
| P49W21G30 | 49% | 21% | 30% |
| P65W20G15 | 65% | 20% | 15% |
| P71W19G10 | 71% | 19% | 10% |

### Example 3: Generating Recombinant Silk Extrudates with Minimal Degradation—P49W21G30

To assess degradation over a number of different conditions, the recombinant spider silk formulations listed in Example 2 were subject to various temperatures during extrusion and various amounts of pressure and shear force. Specifically, the rotations per minute of the twin screw extruded pellets were varied to provide a variable amount of torque and shear force. Various temperature and RPM combinations used to transform the recombinant spider silk formulation into the melt state and extrude the different samples are included below.

The extruded pellets of the P49W21G30 and P65W20G15 formulation listed in Table 1 were again subject to extrusion at various RPM and temperatures using the Xceptional Instruments TSE. Other parameters for operating the Xceptional Instruments TSE were the same as those described above with respect to Example 2.

As described in Example 2, the P71W19G10 formulation was also extruded at various RPM and temperatures using the Xceptional Instruments TSE. Other parameters for operating the Xceptional Instruments TSE were the same as those described above with respect to Example 2.

Data characterizing the relative amounts of high, low and intermediate molecular weight impurities, monomeric 18B and aggregate 18B was collected using Size Exclusion Chromatography (SEC) as follows: 18B powder was dissolved in 5M Guanidine Thiocyanate and injected onto a Yarra SEC-3000 SEC-HPLC column to separate constituents on the basis of molecular weight. Refractive index was used as the detection modality. 18B aggregates, 18B monomer, low molecular weight (1-8 kDa) impurities, intermediate molecular weight impurities (8-50 kDa) and high molecular weight impurities (110-150 kDa) were quantified. Relevant composition was reported as mass % and area %. BSA was used as a general protein standard with the assumption that>90% of all proteins demonstrate do/dc values (the response factor of refractive index) within ˜7% of each other. Poly(ethylene oxide) was used as a retention time standard, and a BSA calibrator was used as a check standard to ensure consistent performance of the method.

Tables 3-5 below lists the various SEC analyses for the extrudates produced under various RPMs and temperatures. The fifth column includes either the difference in 18B monomer (area %) reported in the starting pellets and extrudates (P49W21G30 and P65W20G15) or the difference in 18B monomer (area %) reported in the starting powder and extrudates (P71W19G10). FIGS. 1-3 are described in detail below and include graphs corresponding to Tables 3-5, respectively. From these it can be seen that degradation is minimal across all temperatures and RPMs tested, indicating a flexibility of processing conditions and a general robustness to processing using extrusion methods.

TABLE 3
SEC analysis for P49W21G30

| | | | | Difference between | | | |
|---|---|---|---|---|---|---|---|
| | | | | 18B monomer % | | | |
| | | | 18B | starting pellets | High | Int. | Low |
| Sample ID | Temp. | RPM | monomer % | and samples | MW | MW | MW |
| P49W21G30-1 | 20° C. | 10 | 48.4 | 10.91 | 1.55 | 33.17 | 10.88 |
| P49W21G30-2 | 20° C. | 100 | 42.53 | 16.78 | 1.81 | 35.82 | 14.14 |
| P49W21G30-3 | 20° C. | 200 | 47.77 | 11.54 | 3.55 | 31.28 | 10.73 |
| P49W21G30-4 | 20° C. | 300 | 43.52 | 15.79 | 1.46 | 35.46 | 14.75 |
| P49W21G30-5 | 40° C. | 10 | 54.78 | 4.53 | 4.69 | 27.53 | 4.2 |
| P49W21G30-6 | 40° C. | 100 | 56.87 | 2.44 | 4.82 | 26.18 | 3.07 |
| P49W21G30-7 | 40° C. | 200 | 53.65 | 5.66 | 4.11 | 27.83 | 6 |
| P49W21G30-8 | 40° C. | 300 | 55.15 | 4.16 | 4.70 | 26.75 | 5.66 |
| P49W21G30-9 | 60° C. | 10 | 52.06 | 7.25 | 4.32 | 28.68 | 7.08 |
| P49W21G30-10 | 60° C. | 100 | 54.46 | 4.85 | 4.27 | 28.65 | 4.93 |
| P49W21G30-11 | 60° C. | 200 | 55.74 | 3.57 | 4.31 | 27.61 | 4.18 |
| P49W21G30-12 | 60° C. | 300 | 54.21 | 5.1 | 3.71 | 28.56 | 4.72 |
| P49W21G30-13 | 80° C. | 10 | 53.78 | 5.53 | 3.73 | 29.2 | 5.19 |
| P49W21G30-14 | 80° C. | 100 | 55.97 | 3.34 | 3.53 | 26.32 | 6.36 |
| P49W21G30-15 | 80° C. | 200 | 53.94 | 5.37 | 3.77 | 28.69 | 5.58 |
| P49W21G30-16 | 80° C. | 300 | 54.02 | 5.29 | 3.50 | 27.65 | 6.99 |
| P49W21G30-17 | 95° C. | 10 | 45.16 | 14.15 | 3.58 | 34.9 | 8.18 |
| P49W21G30-18 | 95° C. | 100 | 55.76 | 3.55 | 2.25 | 28.98 | 5.4 |
| P49W21G30-19 | 95° C. | 200 | 50.2 | 9.11 | 2.17 | 30.64 | 10.53 |
| P49W21G30-20 | 95° C. | 300 | 46.31 | 13 | 2.72 | 32.65 | 11.55 |
| P49W21G30-21 | 120° C. | 10 | 53.91 | 5.4 | 3.68 | 28.35 | 5.88 |
| P49W21G30-22 | 120° C. | 100 | 52.11 | 7.2 | 3.97 | 31.65 | 6.19 |
| P49W21G30-23 | 120° C. | 200 | 48.85 | 10.46 | 2.89 | 31.83 | 10.15 |
| P49W21G30-24 | 120° C. | 300 | 51.09 | 8.22 | 3.51 | 31.37 | 7.8 |

TABLE 4
SEC analysis for P65W20G15

| | | | | Difference between | | | |
|---|---|---|---|---|---|---|---|
| | | | | 18B monomer % | | | |
| | | | 18B | in samples and | High | Int. | Low |
| Sample ID | Temp. | RPM | monomer % | starting pellets | MW | MW | MW |
| P65W20G15-1 | 20° C. | 10 | 53.58 | 5.73 | 3.368 | 30.29 | 4.23 |
| P65W20G15-2 | 20° C. | 100 | 53.76 | 5.55 | 3.514 | 28.89 | 6.17 |
| P65W20G15-3 | 20° C. | 200 | 53 | 6.31 | 3.272 | 30.55 | 5.3 |
| P65W20G15-4 | 20° C. | 300 | 52.62 | 6.69 | 3.558 | 30.28 | 5.63 |
| P65W20G15-5 | 40° C. | 10 | 54.35 | 4.96 | 3.186 | 30.3 | 4.88 |
| P65W20G15-6 | 40° C. | 100 | 53.68 | 5.63 | 4.279 | 27.96 | 4.32 |
| P65W20G15-7 | 40° C. | 200 | 54.13 | 5.18 | 3.462 | 28.44 | 5.48 |
| P65W20G15-8 | 40° C. | 300 | 52.01 | 7.3 | 3.933 | 30.01 | 6.11 |
| P65W20G15-9 | 60° C. | 10 | 55.78 | 3.53 | 3.332 | 27.92 | 5.03 |
| P65W20G15-10 | 60° C. | 100 | 58.05 | 1.26 | 3.814 | 26.08 | 3.55 |
| P65W20G15-11 | 60° C. | 200 | 57.47 | 1.84 | 3.308 | 27.06 | 4.25 |
| P65W20G15-12 | 60° C. | 300 | 58.55 | 0.76 | 2.874 | 26.54 | 3.9 |
| P65W20G15-13 | 95° C. | 10 | 52.02 | 7.29 | 2.47 | 29.51 | 8.32 |
| P65W20G15-14 | 95° C. | 100 | 49.92 | 9.39 | 2.48 | 29.3 | 11.24 |
| P65W20G15-15 | 95° C. | 200 | 44.02 | 15.29 | 1.96 | 32.37 | 15 |
| P65W20G15-16 | 95° C. | 300 | 51.31 | 8 | 1.84 | 31.52 | 8.22 |
| P65W20G15-17 | 140° C. | 10 | 50.49 | 8.82 | 5.53 | 28.04 | 4.6 |
| P65W20G15-18 | 140° C. | 100 | 59.4 | −0.09 | 3.241 | 24.7 | 3.4 |
| P65W20G15-19 | 140° C. | 200 | 54.96 | 4.35 | 4.245 | 27.17 | 3.78 |
| P65W20G15-20 | 140° C. | 300 | 54.85 | 4.46 | 4.353 | 26.14 | 5.12 |

TABLE 5
SEC analysis for P71W19G10

| | | | | Difference between | | | |
|---|---|---|---|---|---|---|---|
| | | | | 18B monomer % | | | |
| | | | 18B | in samples and | High | Int. | Low |
| Sample ID | Temp. | RPM | monomer % | starting powder | MW | MW | MW |
| P71W19G10-1 | 90° C. | 10 | 48.61 | 10.7 | 2.90 | 29.95 | 11.01 |
| P71W19G10-2 | 90° C. | 100 | 55.17 | 4.14 | 2.47 | 28.87 | 5.64 |
| P71W19G10.5-3 | 90° C. | 200 | 42.27 | 17.04 | 3.44 | 34.84 | 11.89 |
| P71W19G10-4 | 90° C. | 300 | 31.41 | 27.9 | 4.02 | 39.24 | 17.53 |
| P71W19G10-5 | 120° C. | 10 | 37.23 | 22.08 | 4.32 | 38.32 | 7.73 |
| P71W19G10-6 | 120° C. | 100 | 33.1 | 26.21 | 5.42 | 38.23 | 8.74 |
| P71W19G10-7 | 120° C. | 200 | 32.61 | 26.7 | 5.01 | 38.46 | 11.38 |
| P71W19G10-8 | 120° C. | 300 | 49.58 | 9.73 | 2.20 | 32.5 | 8.72 |

FIG. 1 shows SEC data for P49W21G30 samples listed above in Table 3 under extrusion conditions at 20, 40, 60, 80, 95 or 120° C., where extrudates were obtained for each temperature using operating parameters of 10, 100, 200 or 300 RPM. 18B monomers (black bars), intermediate molecular weight impurities (grey bars) and low molecular weight impurities (cross hatched bars) are shown as area %.

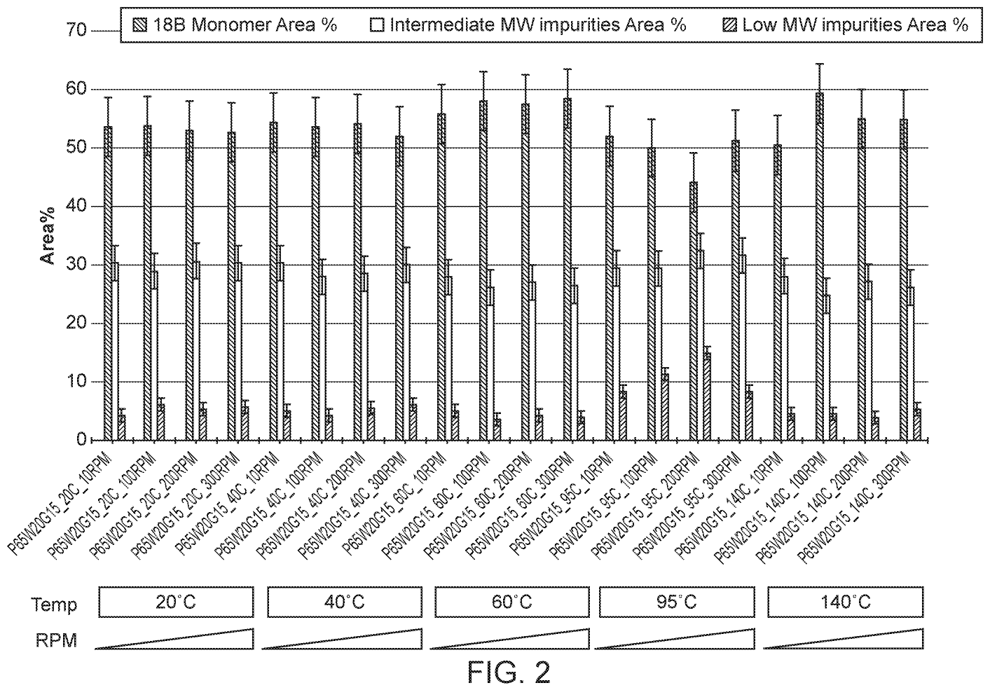

FIG. 2 shows SEC data for P65W20G15 samples listed above in Table 4 under extrusion conditions at 20, 40, 60, 95 or 140° C., where extrudates were obtained for each temperature using operating parameters of 10, 100, 200 or 300 RPM. 18B monomers (black bars), intermediate molecular weight impurities (grey bars) and low molecular weight impurities (cross hatched bars) are shown as area %.

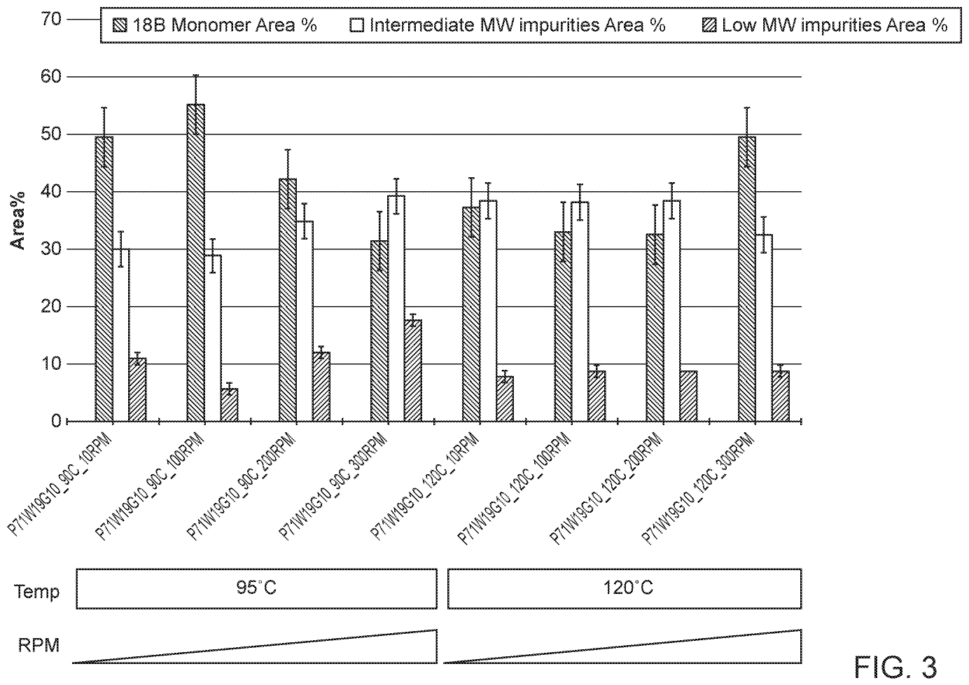

FIG. 3 shows SEC data for P71W19G10 samples listed above in Table 5 under extrusion conditions at 90 or 120° C., where extrudates were obtained for each temperature using operating parameters of 10, 100, 200 or 300 RPM. 18B monomers (black bars), intermediate molecular weight impurities (grey bars) and low molecular weight impurities (cross hatched bars) are shown as area %.

### Example 4: Thermogravimetric Analysis—P49W21G30

In order to analyze water loss during extrusion, the water content of the recombinant spider silk compositions before extrusion and the recombinant spider silk extrudates after extrusion was analyzed by TGA (thermogravimetric analysis) using a TA brand TGA Q500 instrument. For the P49W21G30 and P65W20G15 samples, the water content of the pellets used for the extrusion experiments described in Example 3 was used as a reference sample to measure water loss. For the P71W19G10 samples, the water content of the recombinant spider silk compositions used for the extrusion experiments described in Example 3 was used as a reference sample to measure water loss.

For each sample, 10 mg, +/−1 mg of powders or pellets comprising the formulations listed above were analyzed. To measure water content, samples were run “in air” as opposed to “in nitrogen.” Samples were sequentially introduced into the TGA furnace using the equipped autosampler. The temperature was programmed to increase at a rate of 20° C./minute from room temperature, until it reached 110° C. using the TA brand software suite. The samples were then kept at this temperature for 45 minutes. The samples were then removed from the furnace, and the furnace was flushed with air for 15 minutes before starting the next run.

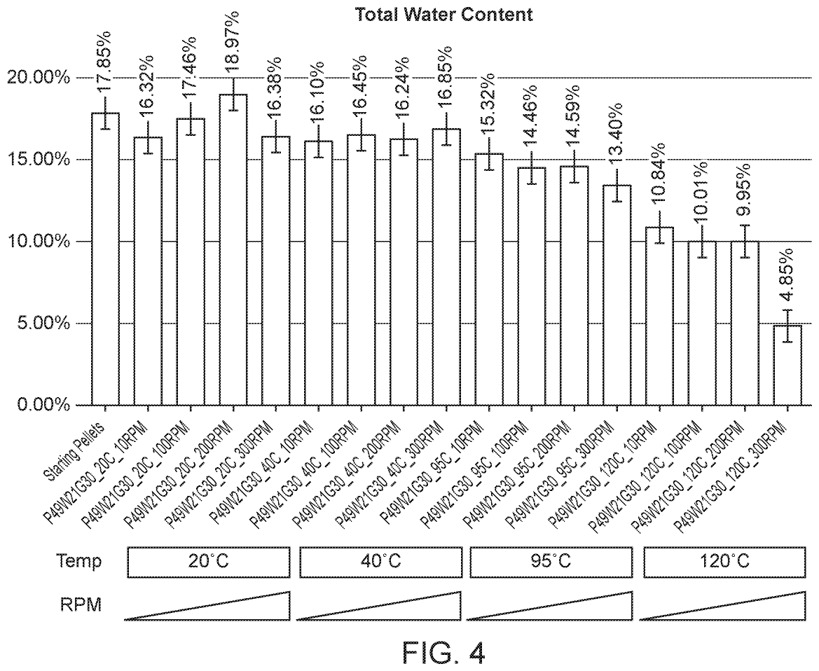

Tables 6-8 below lists the various measurements for the reference samples (i.e. starting pellets or powder) and the extruded samples. FIGS. 4-6 include graphs of the data included in Tables 6-8, respectively. From this data it can be seen that water loss during extrusion is low, and well within acceptable limits for an extrusion process. Typically water loss is in the range 2-18%.

TABLE 6
Water loss in P49W21G30

| | | | Water in | Water | |
|---|---|---|---|---|---|
| | | | Starting | In | Δ |
| Sample ID | Temp. | RPM | Pellets | Extrudates | Water |
| P49W21G30-1 | 20° C. | 10 | 17.95% | 16.32% | 1.63% |
| P49W21G30-2 | 20° C. | 100 | 17.95% | 17.46% | 0.49% |
| P49W21G30-4 | 20° C. | 300 | 17.95% | 16.38% | 1.57% |
| P49W21G30-5 | 40° C. | 10 | 17.95% | 16.10% | 1.85% |
| P49W21G30-6 | 40° C. | 100 | 17.95% | 16.45% | 1.50% |
| P49W21G30-7 | 40° C. | 200 | 17.95% | 16.24% | 1.71% |
| P49W21G30-8 | 40° C. | 300 | 17.95% | 16.85% | 1.10% |
| P49W21G30-9 | 60° C. | 10 | 17.95% | 8.22% | 9.73% |
| P49W21G30-10 | 60° C. | 100 | 17.95% | 11.93% | 6.02% |
| P49W21G30-11 | 60° C. | 200 | 17.95% | 10.59% | 7.36% |
| P49W21G30-12 | 60° C. | 300 | 17.95% | 9.92% | 8.04% |
| P49W21G30-13 | 80° C. | 10 | 17.95% | 9.18% | 8.77% |
| P49W21G30-14 | 80° C. | 100 | 17.95% | 9.08% | 8.87% |
| P49W21G30-15 | 80° C. | 200 | 17.95% | 8.63% | 9.32% |
| P49W21G30-16 | 80° C. | 300 | 17.95% | 8.82% | 9.14% |
| P49W21G30-17 | 95° C. | 10 | 17.95% | 15.32% | 2.63% |
| P49W21G30-18 | 95° C. | 100 | 17.95% | 14.46% | 3.49% |
| P49W21G30-19 | 95° C. | 200 | 17.95% | 14.59% | 3.36% |
| P49W21G30-20 | 95° C. | 300 | 17.95% | 13.40% | 4.55% |
| P49W21G30-21 | 120° C. | 10 | 17.95% | 10.84% | 7.11% |
| P49W21G30-22 | 120° C. | 100 | 17.95% | 10.01% | 7.94% |
| P49W21G30-23 | 120° C. | 200 | 17.95% | 9.95% | 8.00% |
| P49W21G30-24 | 120° C. | 300 | 17.95% | 4.85% | 13.10% |

TABLE 7
Water loss in P65W20G15

| | | | Water in | Water | |
|---|---|---|---|---|---|
| | | | Starting | In | Δ |
| Sample ID | Temp. | RPM | Pellets | Extrudates | Water |
| P65W20G15-1 | 20° C. | 10 | 11.63% | 8.79% | 2.84% |
| P65W20G15-2 | 20° C. | 100 | 11.63% | 8.08% | 3.55% |
| P65W20G15-3 | 20° C. | 200 | 11.63% | 7.78% | 3.85% |
| P65W20G15-4 | 20° C. | 300 | 11.63% | 7.43% | 4.20% |
| P65W20G15-5 | 40° C. | 10 | 11.63% | 7.34% | 4.30% |
| P65W20G15-6 | 40° C. | 100 | 11.63% | 7.07% | 4.56% |
| P65W20G15-7 | 40° C. | 200 | 11.63% | 7.20% | 4.43% |
| P65W20G15-8 | 40° C. | 300 | 11.63% | 7.10% | 4.53% |
| P65W20G15-9 | 60° C. | 10 | 11.63% | 7.17% | 4.46% |
| P65W20G15-10 | 60° C. | 100 | 11.63% | 6.82% | 4.81% |
| P65W20G15-11 | 60° C. | 200 | 11.63% | 6.81% | 4.82% |
| P65W20G15-12 | 60° C. | 300 | 11.63% | 6.47% | 5.16% |
| P65W20G15-16 | 95° C. | 300 | 11.63% | 11.43% | 0.20% |
| P65W20G15-17 | 140° C. | 10 | 11.63% | 6.83% | 4.80% |
| P65W20G15-18 | 140° C. | 100 | 11.63% | 6.22% | 5.41% |

TABLE 8
Water loss in P71W19G10

| | | | Water in | Water | |
|---|---|---|---|---|---|
| | | | Starting | In | Δ |
| Sample ID | Temp. | RPM | Powder | Extrudates | Water |
| P71W19G10-1 | 90° C. | 10 | 7.22% | 7.16% | 0.06% |
| P71W19G10-2 | 90° C. | 100 | 7.22% | 6.84% | 0.38% |
| P71W19G10-3 | 90° C. | 200 | 7.22% | 6.81% | 0.41% |
| P71W19G10-4 | 90° C. | 300 | 7.22% | 6.79% | 0.43% |
| P71W19G10-5 | 120° C. | 10 | 7.22% | 6.21% | 1.01% |
| P71W19G10-6 | 120° C. | 100 | 7.22% | 6.08% | 1.15% |
| P71W19G10-7 | 120° C. | 200 | 7.22% | 5.94% | 1.28% |

FIG. 4 shows TGA data for samples listed above in Table 6 which were generated under extrusion conditions at 20, 40, 95 and 120° C., where extrudates were obtained for each temperature using operating parameters of 10, 100, 200 and 300 RPM. FIG. 4 also shows TGA data for a reference sample of the starting pellets used to generate these samples. The data show % water content of the samples across all treatments, with water loss ranging from ˜1-13% when compared to starting pellets.

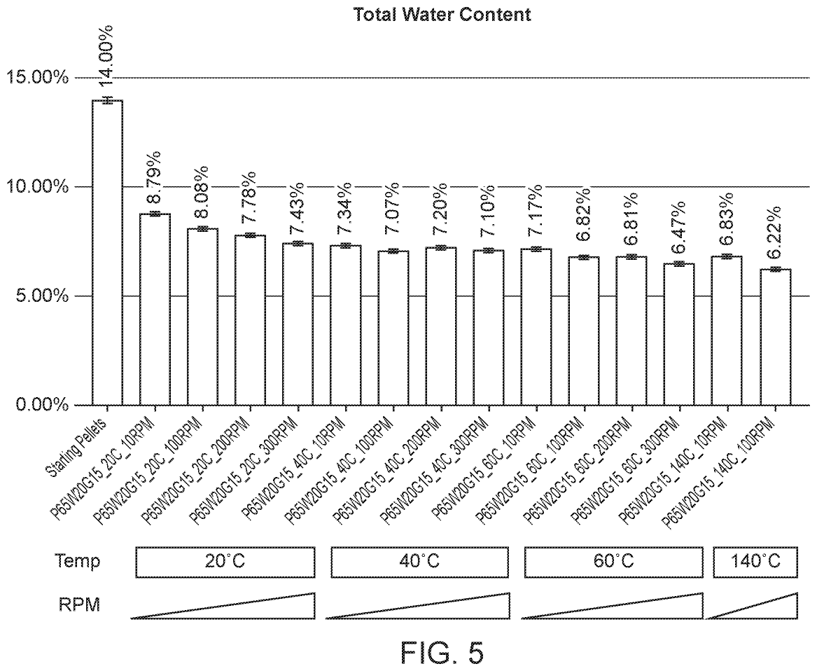

FIG. 5 shows TGA data for samples listed above in Table 7 which were generated under extrusion conditions at 20, 40, 60 and 140° C., where extrudates were obtained for each temperature using operating parameters of 10, 100, 200 and 300 RPM. FIG. 5 also shows TGA data for a reference sample of the starting pellets used to generate these samples. The data show % water content of the samples across all treatments, with water loss ranging from ˜1-8% when compared to starting pellets.

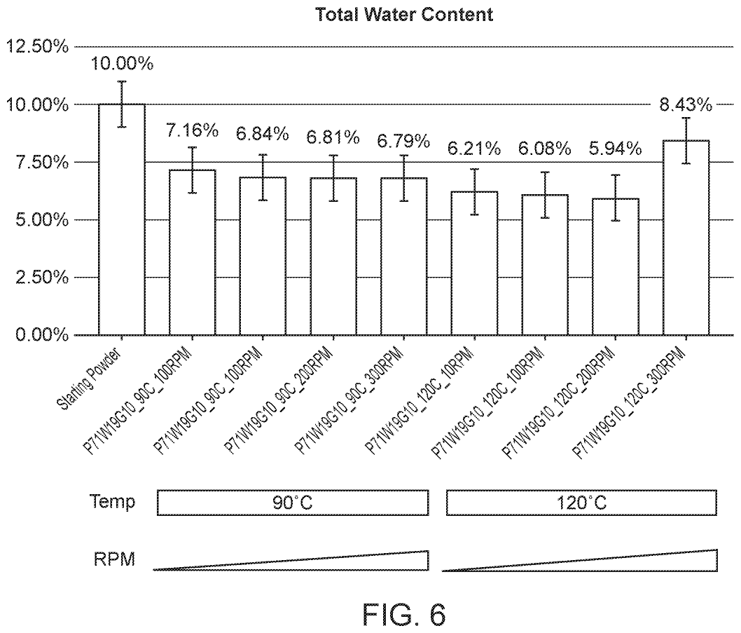

FIG. 6 shows TGA data for samples listed above in Table 8 which were generated under extrusion conditions at 90 and 120° C., where extrudates were obtained for each temperature using operating parameters of 10, 100, 200 and 300 RPM. FIG. 5 also shows TGA data for a reference sample of the starting powder used to generate these samples. The data show % water content of the samples across all treatments, with water loss ranging from ˜1.5-4% when compared to starting powder.

### Example 5: Beta Sheet Content Analysis Using Fourier Transform Infrared Spectroscopy

To assess the formation of secondary and tertiary structures in the extrudates, the beta-sheet content was measured by FTIR (Fourier Transform infrared spectroscopy). FTIR was performed on the extrudates using Bruker Alpha spectrometer equipped with a diamond attenuated total reflection accessory preceded by a wire grid polarizer selecting mostly S (perpendicular) polarized light. Recombinant polypeptide powder and the precursor fiber were included as controls. To quantify the molecular alignment three spectra of each orientation (0 and 90° relative to the polarization electric field) were collected with 32 scans at 4 cm⁻¹ resolution from 4000 to 600 cm⁻¹.

The average values for the peak corresponding to 982-949 cm⁻¹ were calculated based on the following steps. Absorbance values were offset by subtracting the average between 1900 and 1800 cm⁻¹ without bands. Spectra were then normalized by dividing the average between 1350 and 1315 cm⁻¹ corresponding to the isotropic (non-oriented) side chain vibration bands. The beta-sheet content metric was taken to be the average of the integrated absorbance values between 982 and 949 cm⁻¹.

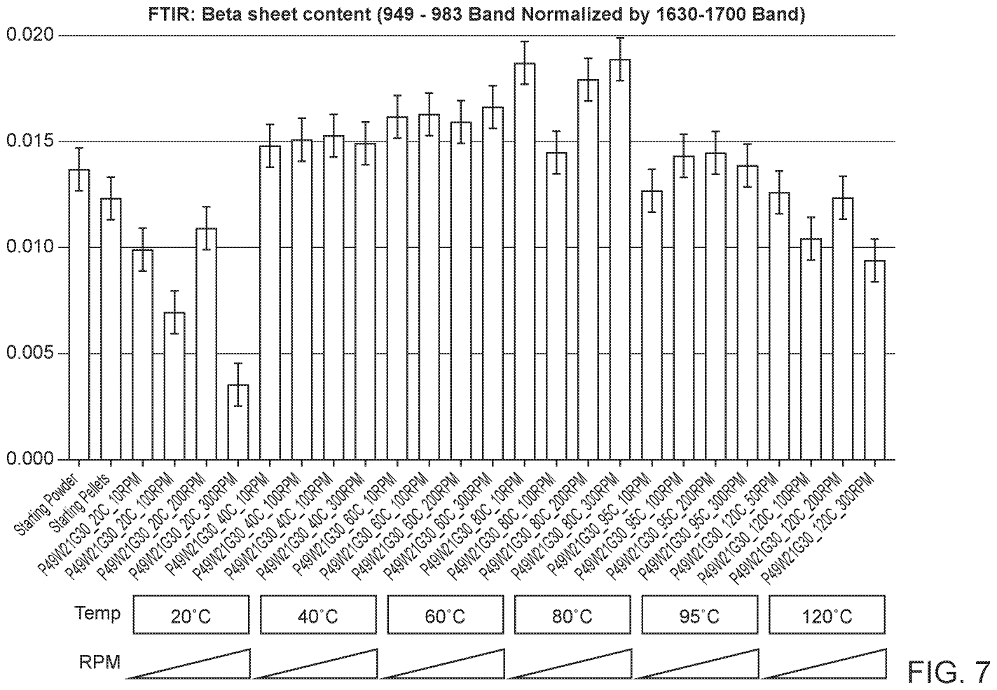

The beta sheet content of the recombinant spider silk extrudates (i.e., “Sample Beta Sheets”) were compared to i) the beta sheet content in the starting recombinant spider silk polypeptide powder used to generate the recombinant spider silk compositions (i.e., “Reference Pre-hydrated Powder”), and ii) the beta sheet content in the starting pellets (P49W21G30 and P65W20G15) (i.e., “Reference Pellets”) Tables 9-11 below lists the measurements for the reference samples and the extrudates produced under the conditions tabulated below. FIGS. 7-9 include graphs of the data shown in Tables 9-11. As can be seen, there is no significant change in the beta-sheet content of the materials from starting recombinant silk polypeptide powder to recombinant spider silk extrudate, indicating that this method enables plasticization and mobility of the amorphous protein domains without disruption to the beta-sheets as would be the case if solvent processing were used.

TABLE 9
Beta Sheet Formation in P49W21G30

| | | | Reference | Reference | |
|---|---|---|---|---|---|
| | | | Pre-hydrated | Pellets | Sample |
| | | | Powder Beta | Beta | Beta |
| | | | Sheets ~982- | Sheets ~982- | Sheets ~982- |
| Sample ID | Temp. | RPM | 949 nm | 949 nm | 949 nm |
| P49W21G30-1 | 20° C. | 10 | 0.01194 | .01229 | 0.009923 |
| P49W21G30-2 | 20° C. | 100 | 0.01194 | .01229 | 0.006975 |
| P49W21G30-3 | 20° C. | 200 | 0.01194 | .01229 | 0.010909 |
| P49W21G30-4 | 20° C. | 300 | 0.01194 | .01229 | 0.003502 |
| P49W21G30-5 | 40° C. | 10 | 0.01194 | .01229 | 0.014843 |
| P49W21G30-6 | 40° C. | 100 | 0.01194 | .01229 | 0.015117 |
| P49W21G30-7 | 40° C. | 200 | 0.01194 | .01229 | 0.015277 |
| P49W21G30-8 | 40° C. | 300 | 0.01194 | .01229 | 0.014973 |
| P49W21G30-9 | 60° C. | 10 | 0.01194 | .01229 | 0.016206 |
| P49W21G30-10 | 60° C. | 100 | 0.01194 | .01229 | 0.016281 |
| P49W21G30-11 | 60° C. | 200 | 0.01194 | .01229 | 0.015997 |
| P49W21G30-12 | 60° C. | 300 | 0.01194 | .01229 | 0.016674 |
| P49W21G30-13 | 80° C. | 10 | 0.01194 | .01229 | 0.018788 |
| P49W21G30-14 | 80° C. | 100 | 0.01194 | .01229 | 0.014512 |
| P49W21G30-15 | 80° C. | 200 | 0.01194 | .01229 | 0.017957 |
| P49W21G30-16 | 80° C. | 300 | 0.01194 | .01229 | 0.018933 |
| P49W21G30-17 | 95° C. | 10 | 0.01194 | .01229 | 0.012738 |
| P49W21G30-18 | 95° C. | 100 | 0.01194 | .01229 | 0.014334 |
| P49W21G30-19 | 95° C. | 200 | 0.01194 | .01229 | 0.014475 |
| P49W21G30-20 | 95° C. | 300 | 0.01194 | .01229 | 0.013899 |
| P49W21G30-21 | 120° C. | 10 | 0.01194 | .01229 | 0.012653 |
| P49W21G30-22 | 120° C. | 100 | 0.01194 | .01229 | 0.010467 |
| P49W21G30-23 | 120° C. | 200 | 0.01194 | .01229 | 0.012384 |
| P49W21G30-24 | 120° C. | 300 | 0.01194 | .01229 | 0.009402 |

TABLE 10
Beta Sheet Formation in P65W20G15

| | | | Reference | Reference | Sample |
|---|---|---|---|---|---|
| | | | Powder Beta | Pellets Beta | Beta |
| | | | Sheets ~982- | Sheets ~982- | Sheets ~982- |
| Sample ID | Temp. | RPM | 949 nm | 949 nm | 949 nm |
| P65W20G15-1 | 20° C. | 10 | 0.02411 | .01719 | 0.01802 |
| P65W20G15-2 | 20° C. | 100 | 0.02411 | .01719 | 0.02023 |
| P65W20G15-3 | 20° C. | 200 | 0.02411 | .01719 | 0.02022 |
| P65W20G15-4 | 20° C. | 300 | 0.02411 | .01719 | 0.01838 |
| P65W20G15-5 | 40° C. | 10 | 0.02411 | .01719 | 0.02021 |
| P65W20G15-6 | 40° C. | 100 | 0.02411 | .01719 | 0.01945 |
| P65W20G15-7 | 40° C. | 200 | 0.02411 | .01719 | 0.01955 |
| P65W20G15-8 | 40° C. | 300 | 0.02411 | .01719 | 0.02083 |
| P65W20G15-9 | 60° C. | 10 | 0.02411 | .01719 | 0.02292 |
| P65W20G15-10 | 60° C. | 100 | 0.02411 | .01719 | 0.01776 |
| P65W20G15-11 | 60° C. | 200 | 0.02411 | .01719 | 0.01926 |
| P65W20G15-12 | 60° C. | 300 | 0.02411 | .01719 | 0.01924 |
| P65W20G15-13 | 95° C. | 10 | 0.02411 | .01719 | 0.01971 |
| P65W20G15-14 | 95° C. | 100 | 0.02411 | .01719 | 0.01905 |
| P65W20G15-15 | 95° C. | 200 | 0.02411 | .01719 | 0.01980 |
| P65W20G15-16 | 95° C. | 300 | 0.02411 | .01719 | 0.02094 |
| P65W20G15-17 | 140° C. | 10 | 0.02411 | .01719 | 0.01956 |
| P65W20G15-18 | 140° C. | 100 | 0.02411 | .01719 | 0.01936 |
| P65W20G15-19 | 140° C. | 200 | 0.02411 | .01719 | 0.01914 |
| P65W20G15-20 | 140° C. | 300 | 0.02411 | .01719 | 0.01863 |

TABLE 11
Beta Sheet Formation in P71W19G10

| | | | Reference | Sample |
|---|---|---|---|---|
| | | | Powder Beta | Beta |
| | | | Sheets ~982- | Sheets ~982- |
| Sample ID | Temp. | RPM | 949 nm | 949 nm |
| P71W19G10-1 | 90° C. | 10 | 0.02411 | 0.02174 |
| P71W19G10-2 | 90° C. | 100 | 0.02411 | 0.01889 |
| P71W19G10-3 | 90° C. | 200 | 0.02411 | 0.02161 |
| P71W19G10-4 | 90° C. | 300 | 0.02411 | 0.01925 |
| P71W19G10-5 | 120° C. | 10 | 0.02411 | 0.02113 |
| P71W19G10-6 | 120° C. | 100 | 0.02411 | 0.02329 |
| P71W19G10-7 | 120° C. | 200 | 0.02411 | 0.02258 |
| P71W19G10-8 | 120° C. | 300 | 0.02411 | 0.02107 |

FIG. 7 shows FTIR data for samples listed above in Table 9 generated under extrusion conditions at 20, 40, 60, 80, 95 or 120° C., where extrudates were obtained for each temperature using operating parameters of 10, 100, 200 or 300 RPM. The data was extracted from the 949-982 and show no clear trends compared to starting pellets.

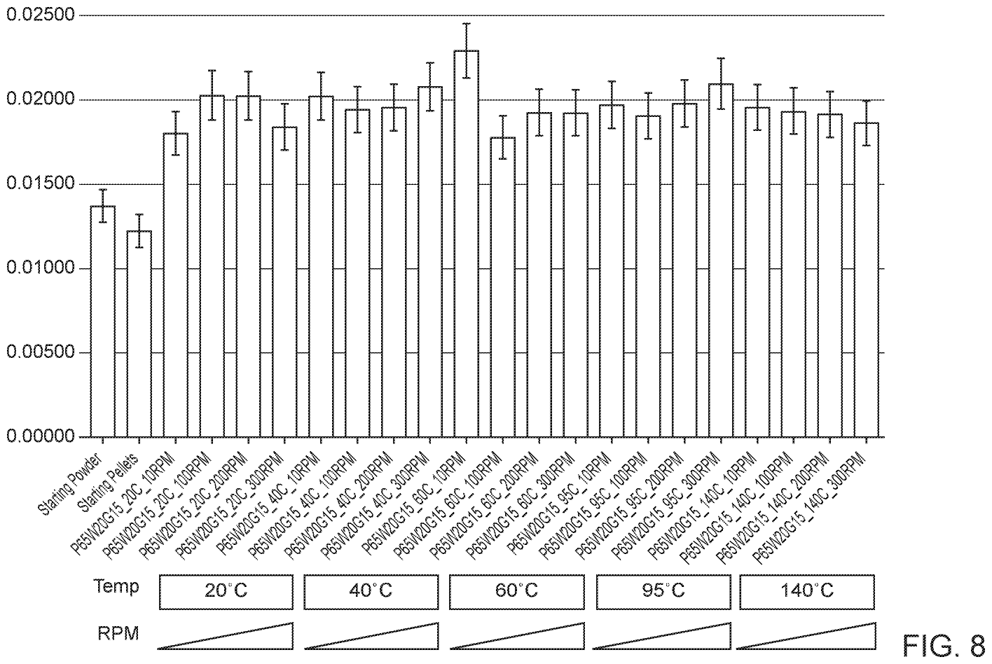

FIG. 8 shows FTIR data for samples for samples listed above in Table 10 which were generated under extrusion conditions at 20, 40, 60, 95 or 140° C., where extrudates were obtained for each temperature using operating parameters of 10, 100, 200 or 300 RPM. The data was extracted from the 949-982 band and show no clear trends compared to starting pellets

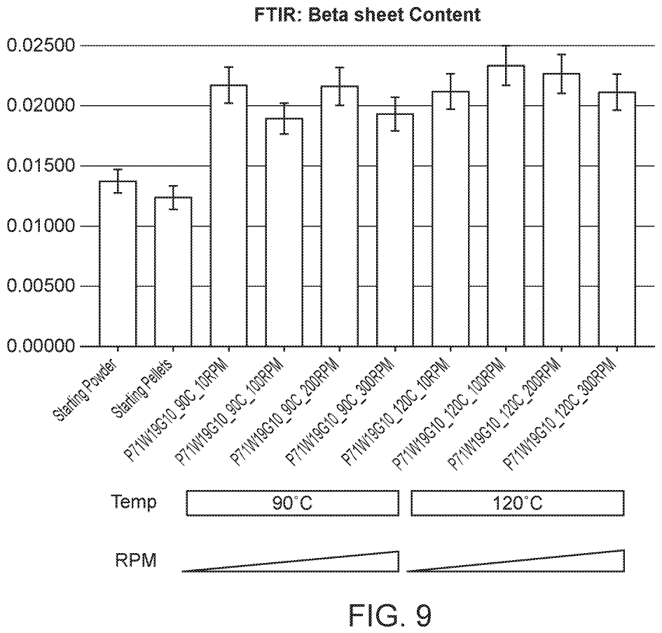

FIG. 9 shows FTIR data for samples for samples listed above in Table 11 which were generated under extrusion conditions at 90 or 120° C., where extrudates were obtained for each temperature using operating parameters of 10, 100, 200 or 300 RPM. The data was extracted from the 949-982 band to avoid artifacts incurred by the presence of water, and show no clear trends compared to starting pellets.

### Example 6: Polarized Light Microscopy—P49W21G30

Polarized Light Microscopy (PL) was used to examine the smoothness and homogeneity of the various extrudates. Light and Polarized Light (PL) images were obtained using a Leica DM750P polarized light microscope, using a 4× PL objective. The Microscope was coupled to the complementary PC based image analysis Leica Application Suite, LAS V4.9. ˜20-30 mm long TSE extrudates were carefully placed along the long axis of standard microscope slides and placed horizontally (East-West; i.e. 0°) above the microscope aperture. Sample edges were initially brought into focus, followed by overall focusing of the sample. The samples were initially viewed under white light, controlled by the illumination control knob, and images captured with the appropriate scale bars included. In all cases the auto-brightness feature of the LAS V4.9 software was switched to off.

Next, the Analyzer/Bertrand Lens module was engaged by flipping the lower rocker of the module to the right (the “A” position/Analyzer in), while ensuring the upper rocker of the Analyzer/Bertrand Lens Module was flipped to the left (the “O” position/Bertrand Lens out). This set up allows for analysis in “cross-polarization mode” which is a state of optical alignment in which the allowed oscillatory directions of the light passing through the polarizer and analyzer are oriented at 90°.

In order to control for background fluctuations in light intensity, all samples were initially viewed, and the brightness of the background was reduced with the illumination control knob until it just reached complete blackness. Each of the eyepieces was then covered with an eyepiece light-blocking accessory to prevent ambient light from passing through during the image capture sequence. Images were captured using the LAS V4.9 software package at 0° and 45° orientations. The 45° images where obtained by rotating the glass side to a 45° angle using the circular rotating stage that this microscope is equipped with.

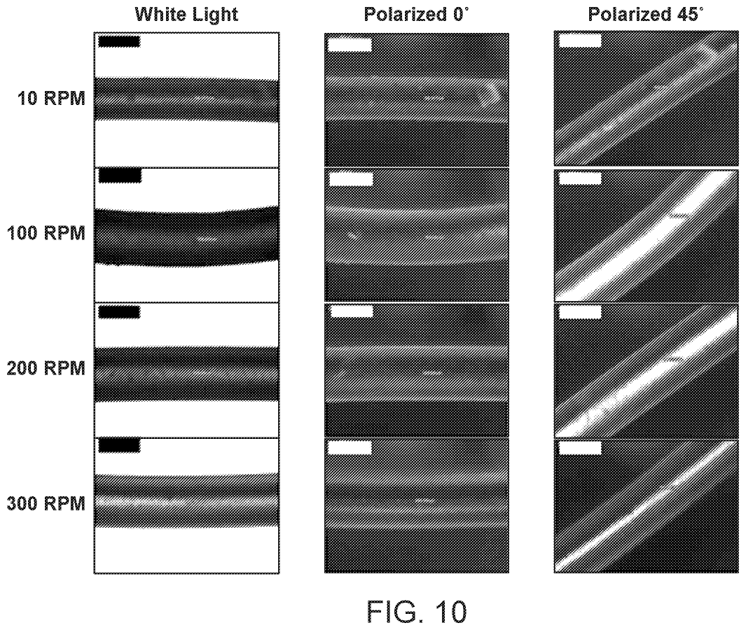

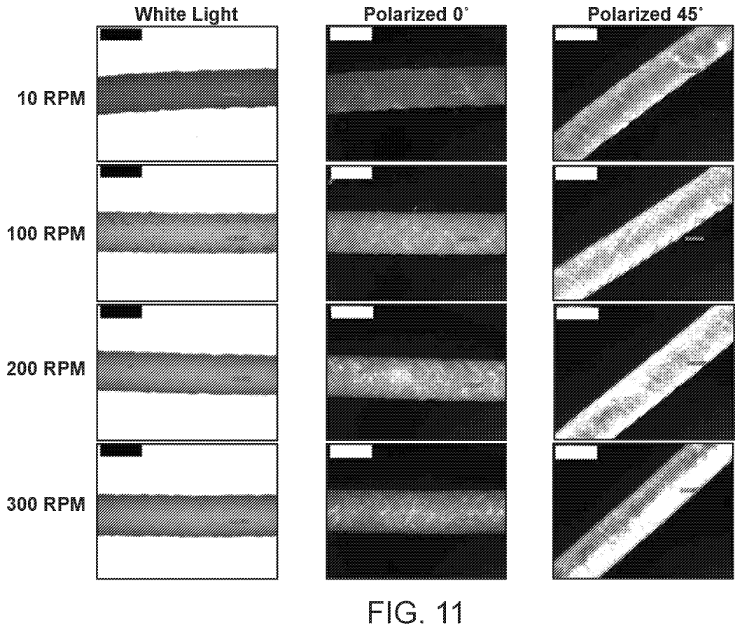

FIGS. 10 and 11 are images of the exemplary samples captured using polarized light microscopy. These show that fibers that are smooth with low melt fracture can be obtained using the claimed processes. Conditions are therefore clearly suitable for melt flow and extrusion. In addition, under many conditions qualitative birefringence was observed, as was axial alignment.

FIG. 10 shows pictures produced from samples P49W21G30-1, P49W21G30-2, P49W21G30-3 and P49W21G30-4 all of which were produced at 20° C. with varying RPMS. Under these conditions the extrudates were smooth with low melt fracture. Polarized Light Microscopy shows preferential axial alignment depending on conditions (examine 45° for differences), where 100 RPM yielded the greatest axial alignment.

FIG. 11 shows pictures produced from samples P49W21G30-17, P49W21G30-18, P49W21G30-19 and P49W21G30-20 all of which were produced at 95° C. with varying RPMS. The extrudates showed moderate melt fracture/surface imperfections. Polarized Light Microscopy showed an increase in axial alignment from 10-100 RPM. From 100-300 RPM the samples showed similar distinction to one another when examined at 0 and 45°.

### Example 7: Metabolites Analysis of Glycerol Content

In order to determine the loss of glycerol from the recombinant spider silk composition during extrusion, the glycerol content was analyzed using a Benson Polymeric 150×7.8 mm H+7110-0 HPLC column equipped with a Phenomenex Security Guard Carbo H+Guard Column, was used with a mobile phase of 0.004 M sulfuric acid. Glycerol calibrants were initially run to enable quantitation. In order to measure the amount of glycerol in the 18B based samples, glycerol present in the compositions was measured before (i.e. as pellets or powder) and after extrusion. For each sample, 25 mg of powder or pellets was dissolved in 1 ml of 0.004 M Sulfuric Acid, and sonicated for 1 hr. The samples were then vortexed and placed in HPLC vials for subsequent runs for each condition/treatment.

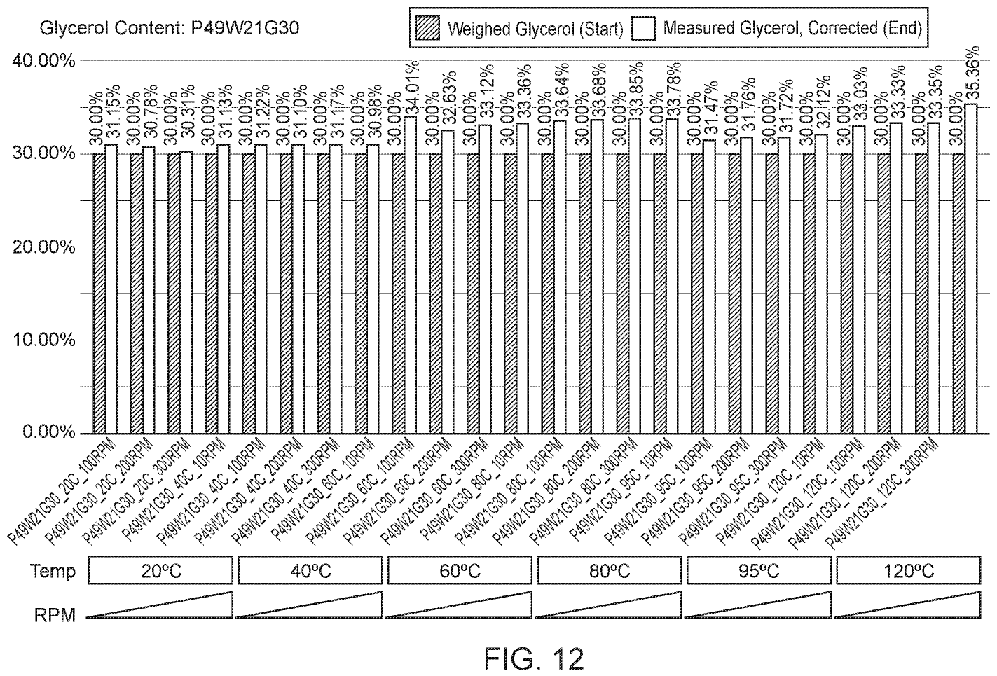

Tables 12-14 below list the various measurements for the extrudates produced under the conditions tabulated below. FIGS. 12-14 include graphs of the same samples. From these it can be seen that glycerol content in the compositions is stable across the range of conditions tested, as evidenced by minimal loss during testing.

TABLE 12
Glycerol Loss in Extrudates - P49W21G30

| | | | | Glycerol | | |
|---|---|---|---|---|---|---|
| | | | Weighed | Concentration, | Measured | |
| | | | Glycerol | corrected for | Glycerol | Δ |
| Sample ID | Temp. | RPM | Wt. % | water loss | Concentration | Glycerol |
| P49W21G30-1 | 20° C. | 10 | 30% | 31.15% | 38.99% | 1.15% |
| P49W21G30-2 | 20° C. | 100 | 30% | 30.78% | 39.14% | 0.78% |
| P49W21G30-3 | 20° C. | 200 | 30% | 30.31% | 39.28% | 0.31% |
| P49W21G30-4 | 20° C. | 300 | 30% | 31.13% | 39.37% | 1.13% |
| P49W21G30-5 | 40° C. | 10 | 30% | 31.22% | 32.74% | 1.22% |
| P49W21G30-6 | 40° C. | 100 | 30% | 31.10% | 33.16% | 1.10% |
| P49W21G30-7 | 40° C. | 200 | 30% | 31.17% | 32.90% | 1.17% |
| P49W21G30-8 | 40° C. | 300 | 30% | 30.98% | 32.90% | 0.98% |
| P49W21G30-9 | 60° C. | 10 | 30% | 34.01% | 32.87% | 4.01% |
| P49W21G30-10 | 60° C. | 100 | 30% | 32.63% | 33.36% | 2.63% |
| P49W21G30-11 | 60° C. | 200 | 30% | 33.12% | 32.90% | 3.12% |
| P49W21G30-12 | 60° C. | 300 | 30% | 33.36% | 33.23% | 3.36% |
| P49W21G30-13 | 80° C. | 10 | 30% | 33.64% | 33.29% | 3.64% |
| P49W21G30-14 | 80° C. | 100 | 30% | 33.68% | 33.65% | 3.68% |
| P49W21G30-15 | 80° C. | 200 | 30% | 33.85% | 34.24% | 3.85% |
| P49W21G30-16 | 80° C. | 300 | 30% | 33.78% | 33.44% | 3.78% |
| P49W21G30-17 | 95° C. | 10 | 30% | 31.47% | 39.85% | 1.47% |
| P49W21G30-18 | 95° C. | 100 | 30% | 31.76% | 39.99% | 1.76% |
| P49W21G30-19 | 95° C. | 200 | 30% | 31.72% | 39.65% | 1.72% |
| P49W21G30-20 | 95° C. | 300 | 30% | 32.12% | 40.28% | 2.12% |
| P49W21G30-21 | 100° C. | 10 | 30% | 33.03% | 33.44% | 3.03% |
| P49W21G30-22 | 100° C. | 100 | 30% | 33.33% | 34.22% | 3.33% |
| P49W21G30-23 | 100° C. | 200 | 30% | 33.35% | 34.94% | 3.35% |
| P49W21G30-24 | 100° C. | 300 | 30% | 35.36% | 34.72% | 5.36% |

TABLE 13
Glycerol Loss in Extrudates - P65W20G15

| | | | | Glycerol | | |
|---|---|---|---|---|---|---|
| | | | Weighed | Concentration, | Measured | |
| | | | Glycerol | corrected for | Glycerol | Δ |
| Sample ID | Temp. | RPM | Wt. % | water loss | Concentration | Glycerol |
| P65W20G15-1 | 20° C. | 10 | 15% | 16.89% | 16.88% | 1.89% |
| P65W20G15-2 | 20° C. | 100 | 15% | 17.03% | 16.77% | 2.03% |
| P65W20G15-3 | 20° C. | 200 | 15% | 17.09% | 16.97% | 2.09% |
| P65W20G15-4 | 20° C. | 300 | 15% | 17.16% | 16.88% | 2.16% |
| P65W20G15-5 | 40° C. | 10 | 15% | 17.18% | 17.26% | 2.18% |
| P65W20G15-6 | 40° C. | 100 | 15% | 17.23% | 17.17% | 2.23% |
| P65W20G15-7 | 40° C. | 200 | 15% | 17.20% | 17.44% | 2.20% |
| P65W20G15-8 | 40° C. | 300 | 15% | 17.22% | 17.55% | 2.22% |
| P65W20G15-9 | 60° C. | 10 | 15% | 17.21% | 17.61% | 2.21% |
| P65W20G15-10 | 60° C. | 100 | 15% | 17.28% | 17.48% | 2.28% |
| P65W20G15-11 | 60° C. | 200 | 15% | 17.28% | 17.69% | 2.28% |
| P65W20G15-12 | 60° C. | 300 | 15% | 17.35% | 17.57% | 2.35% |
| P65W20G15-13 | 95° C. | 10 | 15% | 15.66% | 21.73% | 0.66% |
| P65W20G15-14 | 95° C. | 100 | 15% | 15.66% | 20.53% | 0.66% |
| P65W20G15-15 | 95° C. | 200 | 15% | 15.72% | 20.29% | 0.72% |
| P65W20G15-16 | 95° C. | 300 | 15% | 16.41% | 21.43% | 1.41% |
| P65W20G15-17 | 140° C. | 10 | 15% | 17.27% | 18.06% | 2.27% |
| P65W20G15-18 | 140° C. | 100 | 15% | 17.40% | 18.00% | 2.40% |
| P65W20G15-19 | 140° C. | 200 | 15% | 16.04% | 18.04% | 1.04% |
| P65W20G15-20 | 140° C. | 300 | 15% | 16.13% | 18.37% | 1.13% |

TABLE 14
Glycerol Loss in Extrudates - P71W19G10

| | | | | Glycerol | | |
|---|---|---|---|---|---|---|
| | | | Weighed | Concentration, | Measured | |
| | | | Glycerol | corrected for | Glycerol | Δ |
| Sample ID | Temp. | RPM | Wt. % | water loss | Concentration | Glycerol |
| P71W19G10-1 | 90° C. | 10 | 10% | 10.82% | 13.86% | 0.82% |
| P71W19G10-2 | 90° C. | 100 | 10% | 10.76% | 13.83% | 0.76% |
| P71W19G10-3 | 90° C. | 200 | 10% | 10.87% | 14.07% | 0.87% |
| P71W19G10-4 | 90° C. | 300 | 10% | 9.58% | 14.09% | −0.42%* |
| P71W19G10-5 | 120° C. | 10 | 10% | 9.63% | 13.62% | −0.37%* |
| P71W19G10-6 | 120° C. | 100 | 10% | 9.58% | 13.64% | −0.42%* |
| P71W19G10-7 | 120° C. | 200 | 10% | 10.14% | 13.68% | 0.14% |
| P71W19G10-8 | 120° C. | 300 | 10% | 10.91% | 14.44% | 0.91% |

*Anomalous result within error range of testing instrument.

FIG. 12 shows Metabolites data for samples listed above in Table 12 generated under extrusion conditions at 20, 40, 60, 80, 95 and 120° C., where extrudates were obtained for each temperature using operating parameters of 10, 100, 200 and 300 RPM. Glycerol loss was negligible across all treatments.

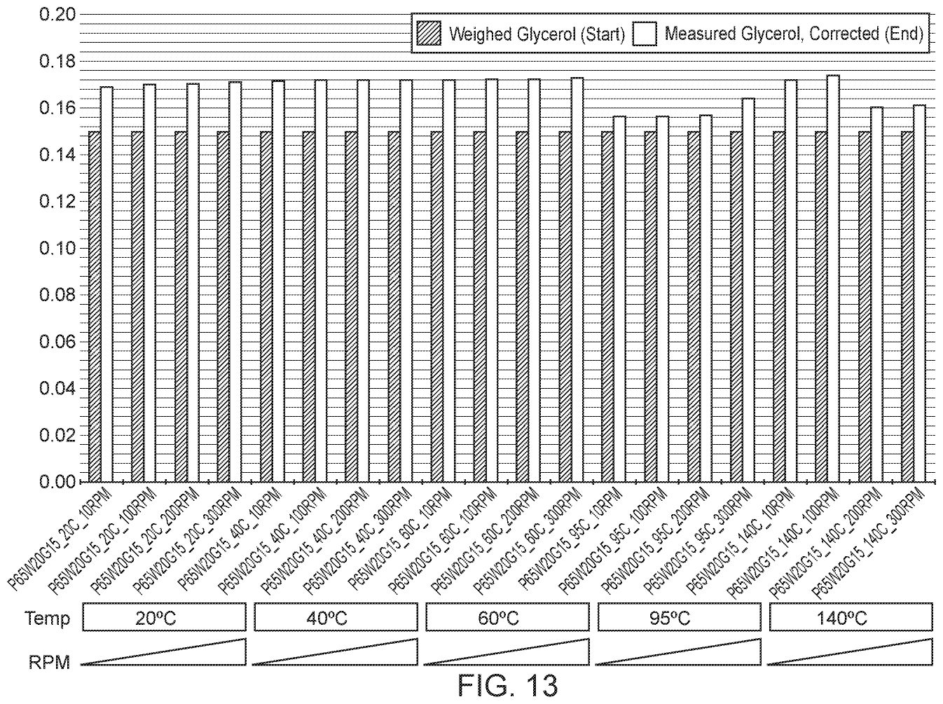

FIG. 13 shows Metabolites data for samples listed above in Table 13 generated under extrusion conditions at 20, 40, 60, 95 and 140° C., where extrudates were obtained for each temperature using operating parameters of 10, 100, 200 and 300 RPM. Glycerol loss was negligible across all treatments.

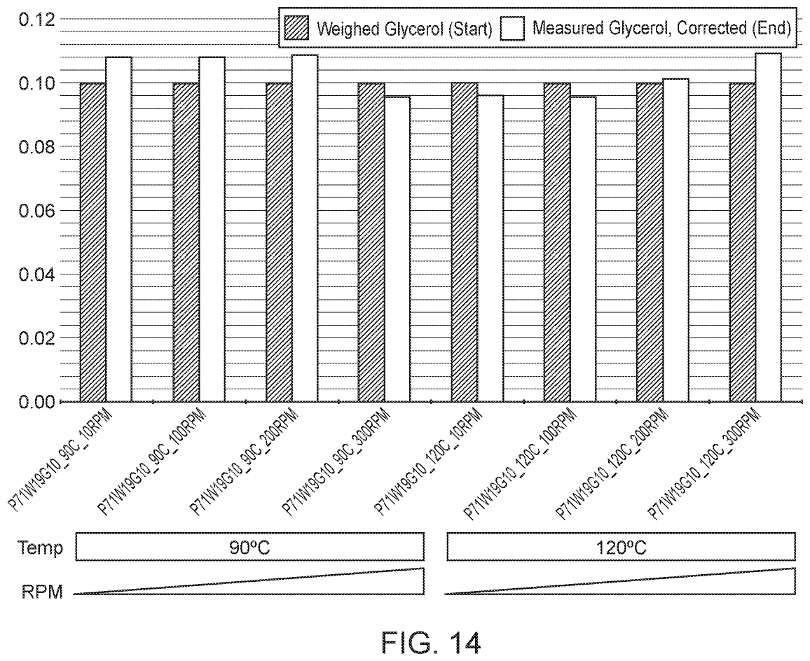

FIG. 14 shows Metabolites data for samples listed above in Table 14 generated under extrusion conditions at 90 and 120° C., where extrudates were obtained for each temperature using operating parameters of 10, 100, 200 and 300 RPM. Glycerol loss was negligible across all treatments.

### Example 8: Microscopic Analysis of Silk Glycerol Extrudates

To investigate the effects of the duration of circulation under shear force and pressure on the morphology of the recombinant spider silk in the extrudate, a recombinant spider silk polypeptide powder similar to that described in Example 1 was mixed with glycerin and subject to different durations of circulation in a Xplore MC 15 conical twin screw extruder (Xplore TSE) at a temperature of 90° C. for various durations of time.

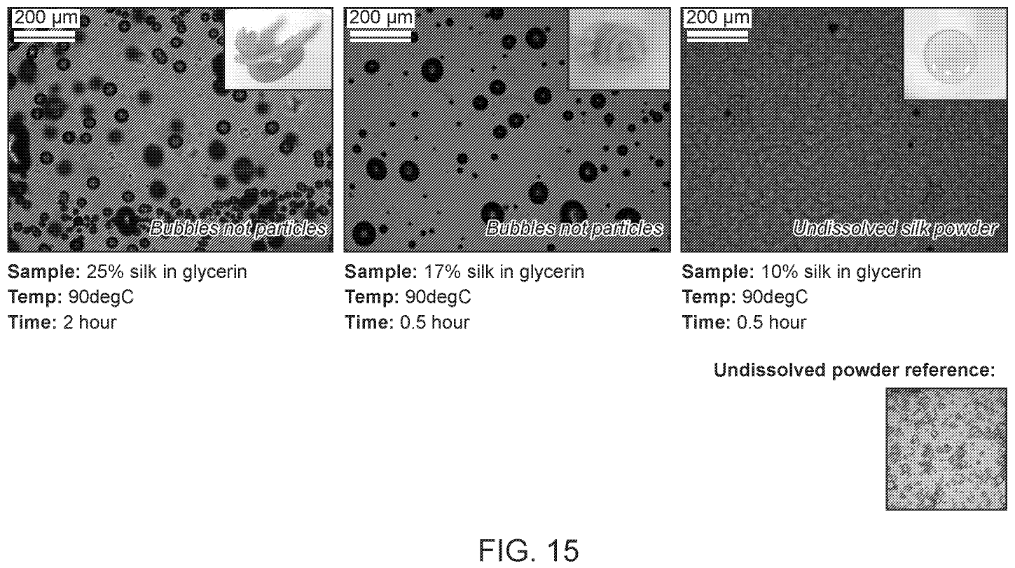

Weight by volume formulations including 10% silk and 90% glycerol (“10% silk”); 17% silk and 83% glycerol (“17% silk”) and 25% silk and 75% glycerol (“25% silk”) were subject to circulation in the XPlore TSE at 90° C. for respective durations of 0.5 hours, 0.5 hours and 2 hours and extruded from the XPlore TSE. The resulting extrudates were examined for morphology of the recombinant spider silk using a Leica 2700M light microscope and a series of visual references of dissolved recombinant spider silk, undissolved recombinant spider silk and recombinant, spider silk powder. FIG. 15 shows extrudate resulting from the above mixtures and methods, as well as an undissolved powder reference (i.e., a mixture of glycerol and silk powder before extrusion). As shown in FIG. 15, the 10% silk extrudate appeared to be undissolved based on comparison to the morphological reference, but the 17% silk extrudate and 25% silk extrudate appeared to be dissolved based on comparison to a morphological reference developed using known standard for undissolved powder.

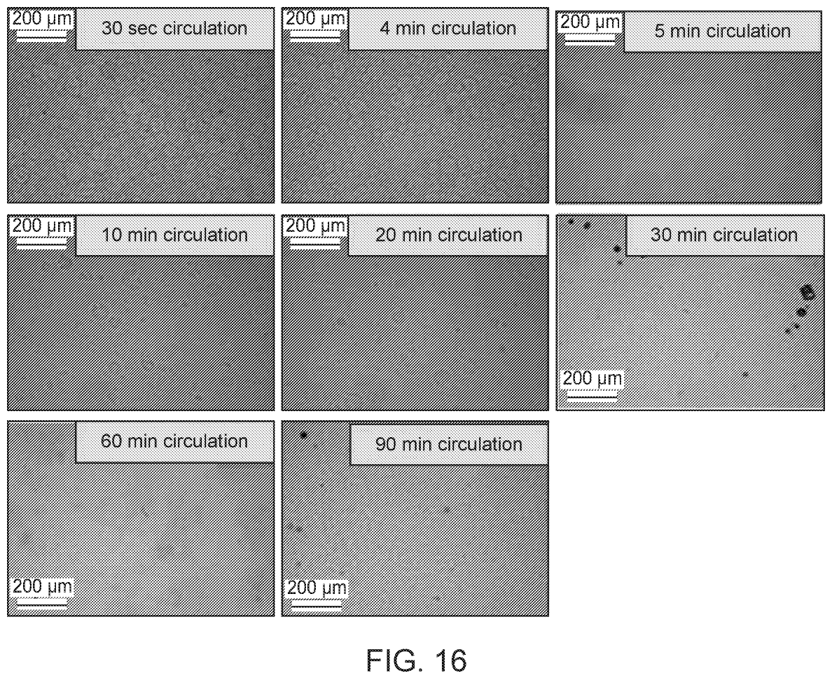

The 25% silk formulation was also circulated in the XPlore TSE at 90° C. for 30 sec, 4 min, 5 min, 10 min, 20 min, 0.5 hours, 1 hour and 1.5 hours and extruded. The extrudate from the various circulation were examined using the Leica 2700M light microscope for any morphological changes due to prolonged circulation. Images from light microscopy examination of each extrudate is shown in FIG. 16. No difference in morphology based on prolonged circulation was observed based on light microscopy.

Various 25% silk formulations using different recombinant spider silk protein powder lots similar in composition to that of Example 1 were analyzed after extrusion for protein degradation using the methods outline above with respect to Example 3. These results are tabulated below in Table 15. As shown in Table 15, minimal degradation was observed with circulation at 90° C. but degradation increased with the duration of circulation.

TABLE 15
18B Aggregates and Monomers in Powder and Extrudates

| | | 18B | 18B | | |
|---|---|---|---|---|---|
| | HMWI Avg | Aggregate | Monomer | IMWI | LMWI |
| Sample | Area % | Avg Area % | Avg Area % | Avg Area % | Avg Area % |
| MB1 153 Powder | 2.77 | 7.383 | 54.16 | 29.78 | 5.81 |
| MBI 153 | 4.53 | 8.64 | 39.77 | 33.31 | 13.76 |
| TSE 2 hour | | | | | |
| extrudate 25% silk | | | | | |
| in glycerin | | | | | |
| MBI 140 Powder | 3.27 | 6.09 | 50.65 | 30.99 | 2.52 |
| MBI 140 | 4.83 | 7.21 | 37.78 | 41.10 | 9.09 |
| TSE 30 min | | | | | |
| extrudate 25% silk | | | | | |
| in glycerin | | | | | |
| MBI 140 | 4.85 | 7.47 | 41.56 | 39.26 | 6.87 |
| TSE 15 min | | | | | |
| extrudate 25% silk | | | | | |
| in glycerin | | | | | |

Solubility of spider silk protein extrudate was assessed as a function of time of circulation during extrusion. Specifically. a mixture of 25% recombinant spider silk polypeptide powder and 75% glycerol was circulated in the above-described twin screw extruder for 30 seconds, 4 minutes, 5 minutes, 10 minutes, 20 minutes, 30 minutes, 60 minutes, and 90 minutes. Each extrudate produced was re-suspended in a mixture of 80% water and 20% extrudate and examined using light microscopy. As shown in FIG. 16, solubility increased over time but did not significantly increase after 30 minutes.

### Example 9: Phase Separation of Recombinant Spider Silk Extrudates

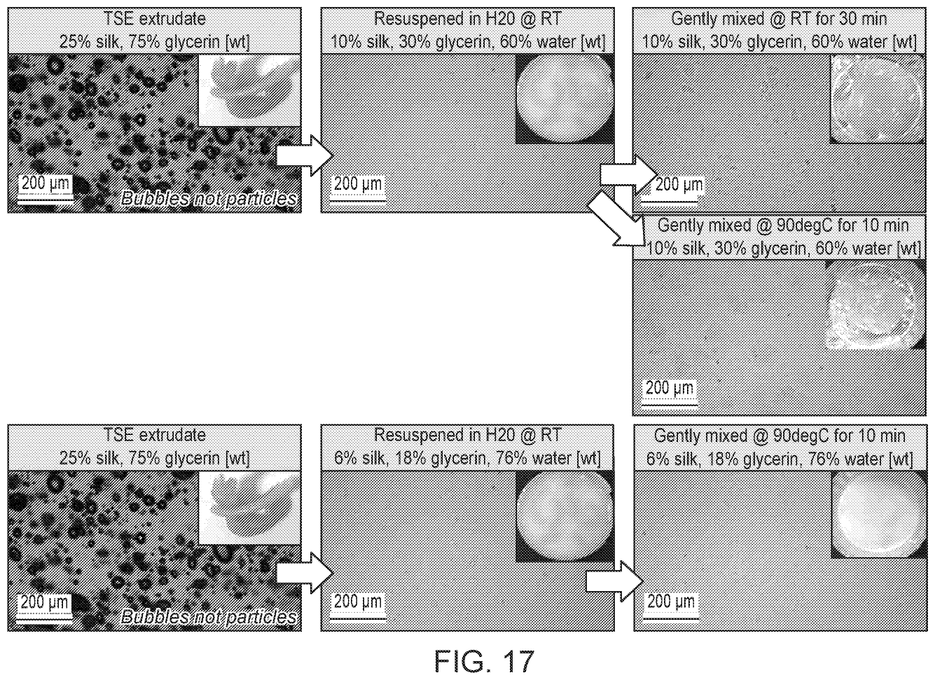

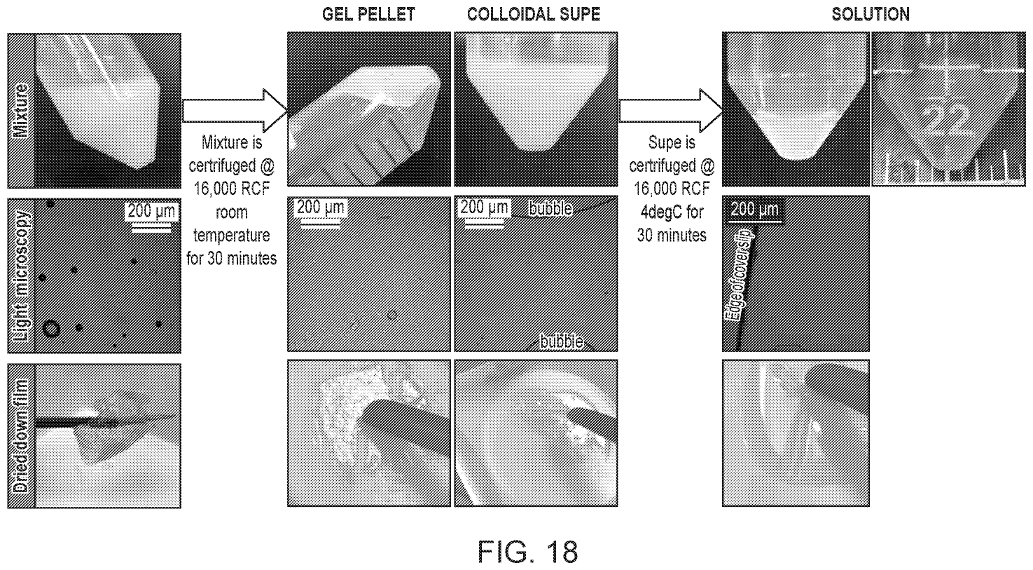

To investigate the properties of the recombinant spider silk extrudate in an aqueous solution, the 25% recombinant spider silk powder and 75% glycerol extrudate described in Example 8 that was circulated for 30 minutes@250 RPM was suspended in various volumes of deionized water and gently broken up by agitation in the water at room temperature (21° C.). In some instances, the extrudate and water suspension was then heated at 90° C. for 10 minutes while agitating the extrudate in the water. FIG. 17 shows morphology analysis using light microscopy as described in Example 8 of an extrudate, an extrudate resuspended in water, and after agitation at room temperature or at 90° C. Specifically, a weight by volume suspension of 60% water and 40% silk extrudate generated a suspension of 10% recombinant silk protein powder, 30% glycerol and 60% water. This suspension was gently agitated for 30 minutes at room temperature or for 10 minutes at 90° C. A weight by volume suspension of 76% water and 24% silk extrudate generated a suspension of 6% recombinant spider silk protein powder, 18% glycerin and 76% water. This suspension was agitated for 10 minutes at 90° C.

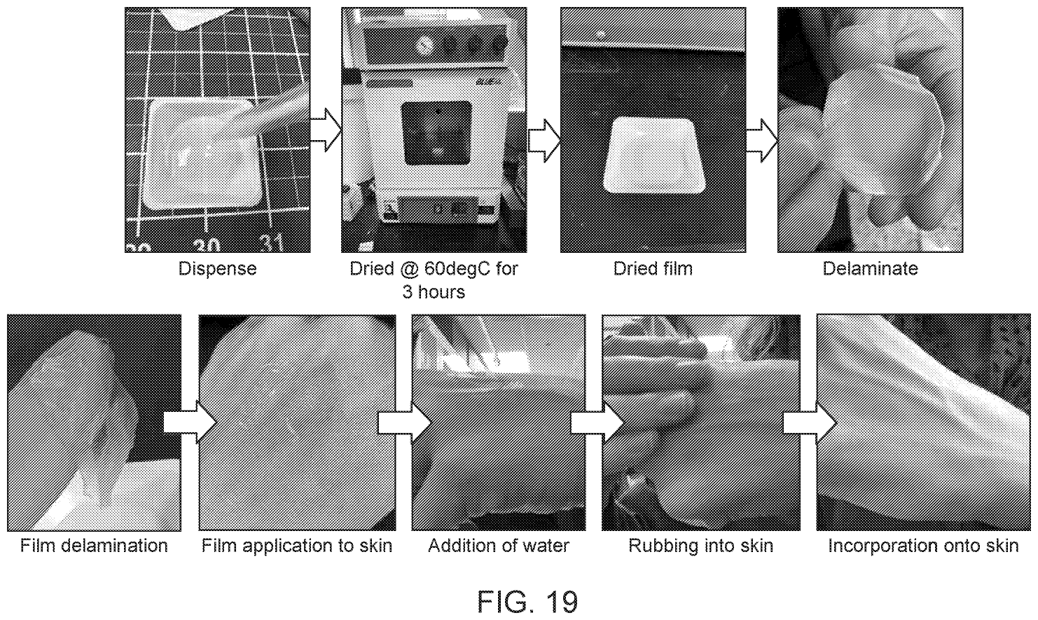

Following heating, the aqueous extrudate suspension comprising the 6% spider silk protein powder, 18% glycerin and 76% water was centrifuged to induce phase separation into 3 distinct phases: a gel phase, a colloid phase, and a solution phase. First, as shown in FIG. 18, centrifugation of the extrudate and water suspension at 16,000 RCF at room temperature for 30 minutes yielded a viscous gel phase that formed a pellet at the bottom of the tube, and a colloidal supernatant phase (comprising a solution phase and a colloid phase) that formed an opaque supernatant that did not settle out over time. A solution phase was obtained by centrifuging the colloidal supernatant phase at 16,000 RCF and 4° C. for 30 minutes to obtain a clear supernatant. The aqueous extrudate suspension, the gel phase, the colloid supernatant phase, and the solution phase were each imaged using light microscopy. A dried down film was formed from each of these phases. The macro view of each phase, an image under light microscopy, and an image of the dried down film generated from each phase is shown in FIG. 18.

### Example 10: Film Formation

To explore the film formation properties of the recombinant spider silk extrudate, the aqueous suspended extrudate was used to make various films.

A “silk-glycerol film” was formed using a recombinant spider silk extrudate made using the process described in Example 8. Specifically a mixture of 25% by weight recombinant spider silk polypeptide powder and 75% by weight glycerol was circulated in a twin-screw extruder for 30 minutes at 90° C. and 250 RPM to generate a recombinant spider silk extrudate. Next, an aqueous suspended extrudate was made by generating a suspension of 20% by weight recombinant spider silk extrudate in 80% by weight deionized water. The suspension of extrudate was gently agitated at 21° C. The aqueous suspended extrudate was then exposed to up to 90° C. for 15 minutes. The heated aqueous suspended extrudate was then cast onto a flat surface and dried at 60° C. under a vacuum of 15 inHg.

A “silk-glycerol emulsion film” was formed using a recombinant spider silk extrudate made using the process described in Example 8. Specifically a mixture of 25% by weight recombinant spider silk polypeptide powder and 75% by weight glycerol was circulated in a twin-screw extruder for 30 minutes at 90° C. and 250 RPM to generate a recombinant spider silk extrudate. The recombinant spider silk extrudate was resuspended in water, agitated, and incorporated into an emulsion with the following ingredients: water, glycerin, pentylene, glycol, silk protein, ceramide AP, ceramide EOP, ceramide NP, sodium hydraluronate, sodium lauroyl lactylate (SLL), cholesterol, xanthan gum, sclerotium gum, lecithin, pullulan, carbomer, hexylene, glycol, ethylhexylglycerin, caprylyl glycol, disodium EDTA, and phenoxyethanol.

The emulsion was then cast onto a flat surface and dried at 60° C. for 4 hours.

A “silk-glycerol emulsion lyophilized film” was formed using a recombinant spider silk extrudate made using the process described in Example 8. Specifically a mixture of 25% by weight recombinant spider silk polypeptide powder and 75% by weight glycerol was circulated in a twin-screw extruder for 30 minutes at 90° C. and 250 RPM to generate a recombinant spider silk extrudate. The recombinant spider silk extrudate was incorporated into an emulsion with the following ingredients: water, glycerin, pentylene, glycol, silk protein, ceramide AP, ceramide EOP, ceramide NP, sodium hydraluronat, sodium lauroyl lactylate (SLL), cholesterol, xantham gum, sclerotium gum, lecithin, pullulan, carbomer, hexylene, glycol, ethylhexylglycerin, caprylyl, glycol disodium EDTA, and phenoxyethanol. The emulsion was then cast onto a flat surface and placed in a Labconco freeze-drier and subjected to −106° C. at 0.008 mBar for 4 hours until the water was sublimated off. The lyophilization resulted in a “spongy” or porous mixture.

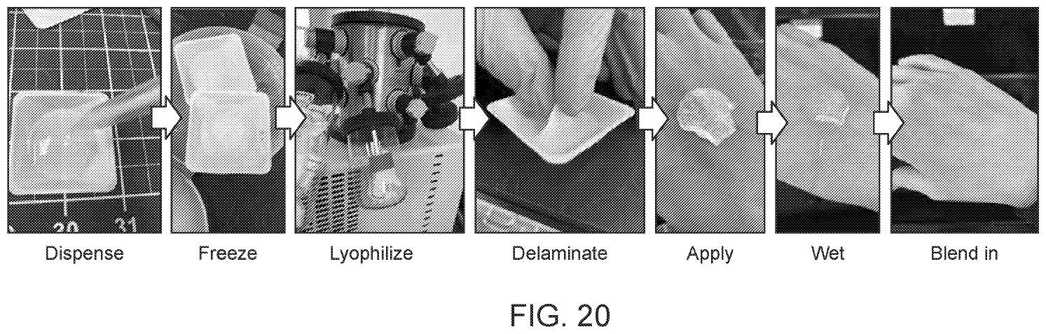

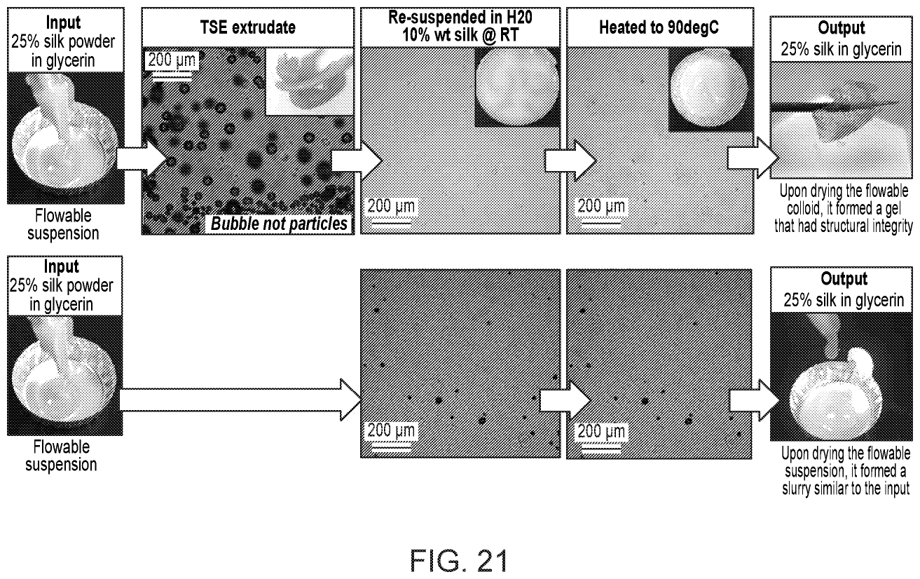

Each of the silk-glycerol film, the silk-glycerol emulsion film and the silk-glycerol emulsion lyophilized film were tested by application to the skin of a test subject. Upon skin contact and application of water, the films formed a dispersible liquid which was adsorbed onto the skin. FIG. 19 shows the above-described process of making the dried silk-glycerol emulsion film and application to the skin of a test subject. FIG. 20 shows steps involved in making the silk-glycerol emulsion lyophilized film and application to the skin of a test subject.

### Example 11: Comparison of Recombinant Spider Silk Extrudate to Non-Extruded Silk-Glycerol Mixtures

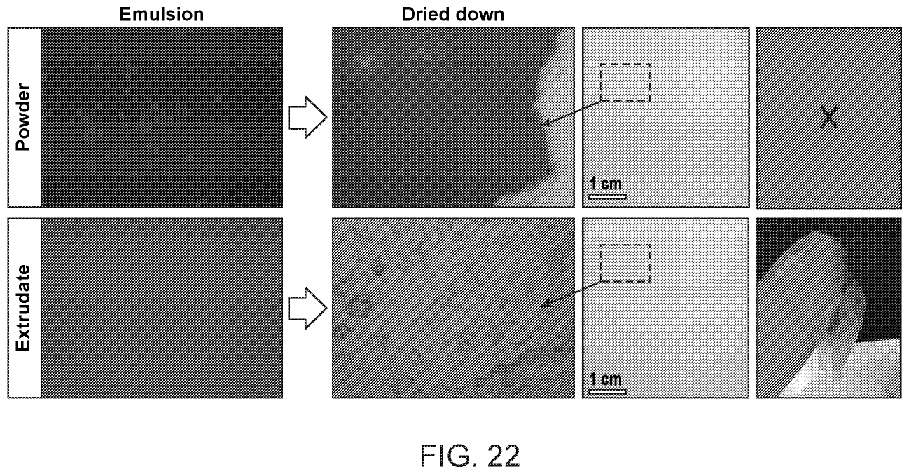

The film-forming capabilities of the recombinant spider silk extrudate was investigated in comparison with non-extruded silk and glycerol mixtures. As a first step, a recombinant spider silk extrudate comprising 25% by weight recombinant spider silk polypeptide powder and 75% by weight glycerol using the method described above in Example 8 was made. Specifically a mixture of 25% by weight recombinant spider silk polypeptide powder and 75% by weight glycerol was circulated in a twin-screw extruder for 30 minutes at 90° C. and 250 RPM to generate a recombinant spider silk extrudate. An aqueous suspended extrudate was made by forming a composition of 10% by weight of the recombinant spider silk extrudate in 90% by weight deionized water. The aqueous suspended extrudate was heated to 90° C., then allowed to dry on a flat surface. As shown in FIG. 21, the dried mixture formed a solid film which could be delaminated from the surface it was cast on.

For comparison, a “slurry” mixture comprising 25% by weight recombinant spider silk polypeptide powder and 75% by weight glycerol was made by mixing the recombinant spider silk polypeptide powder in glycerol to form a viscous slurry. The slurry mixture was suspended in an aqueous solution comprising 90% deionized water and 10% slurry mixture. The suspension of the slurry mixture was then was heated to 90° C., then allowed to dry on a flat surface to investigate whether the slurry mixture would form a film. As shown in FIG. 21, upon drying the aqueous suspension of the slurry mixture, a viscous slurry similar to the slurry mixture before aqueous suspension was observed. Thus, the step of forming the extrudate is beneficial to the film-forming properties of the mixture.

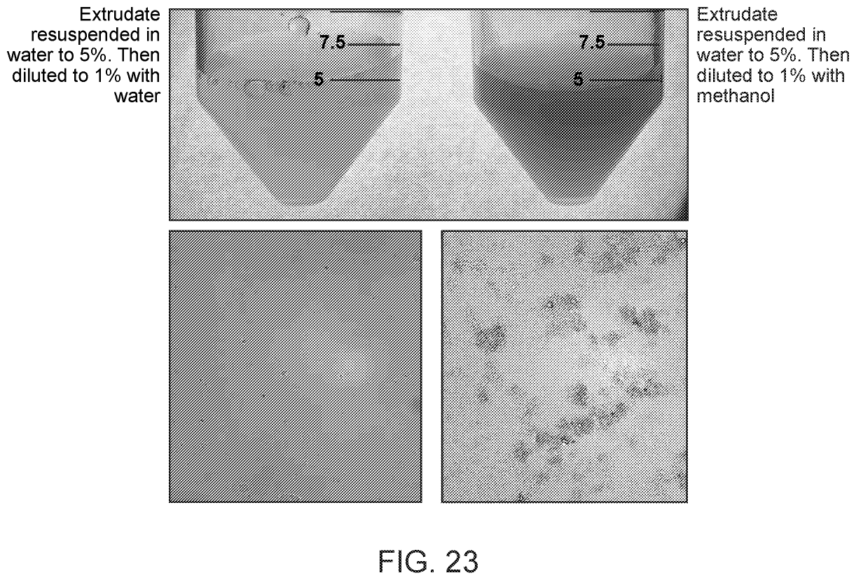

In order to further investigate film forming properties of the mixture, the 25% silk/75% glycerol extrudate and the 25% silk/75% glycerol slurry (non-extrudate) were each added to an emulsion comprising the following ingredients: water, glycerin, pentylene, glycol, silk protein, ceramide AP, ceramide EOP, ceramide NP, sodium hydraluronat, sodium lauroyl lactylate (SLL), cholesterol, xantham gum, sclerotium gum, lecithin, pullulan, carbomer, hexylene, glycol, ethylhexylglycerin, caprylyl, glycol disodium EDTA, and phenoxyethanol. Both formulations were then dried for 4 hours at 60° C. on a flat surface to observe whether film formation could be observed upon drying. As shown in FIG. 22 the formulation comprising an emulsion and the recombinant spider silk extrudate formed a film upon drying. However, no film was observed upon drying the formulation comprising an emulsion and the recombinant spider silk polypeptide powder. Therefore, extrudate formation is beneficial to the film-forming properties of the emulsion mixture.

### Example 12: Silk Extrudate in Methanol

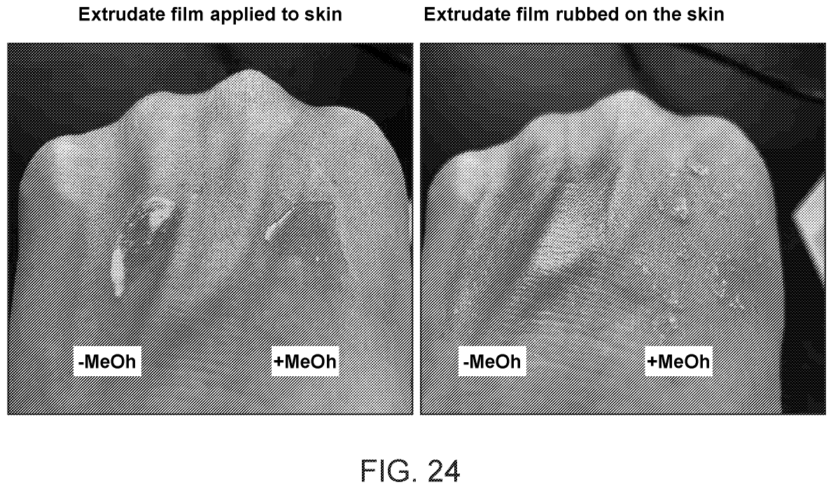

In this experiment, extrudate was prepared by mixing 25% wt powder with 75% wt glycerin and processed through a twin screw extruder at 90° C. at 250 rpm for 30 minutes. The extrudate was then resuspended in water at 5× dilution by gentle mixing at room temperature. This mixture was split in two aliquots. One aliquot was diluted 5× further with water and the second aliquot was diluted 5× with methanol. As shown in FIG. 23, the sample diluted with water did not experience a phase change as determined by visual and microscopic inspection. The sample diluted with methanol experienced a phase change—the mixture became visually more opaque and aggregation was observed microscopically. This result highlights that while the extrudate material and powder have similar FTIR spectroscopic profiles (including similar b-sheet content before and after methanol treatment), the extrudate is unique in that methanol treatment induces aggregation.

Extrudate suspended in water was again prepared and separated into two aliquots as described above. Each aliquot was cast on a flat surface weight boat and dried overnight at ambient room temperature and humidity. This resulted in a thin film material from each aliquot. One film was left untreated and the other was exposed to methanol vapor in a closed chamber overnight. The films were then delaminated and placed on the skin. The untreated film easily rubbed into the skin upon light pressure and shear. The methanol treated film was more mechanically intact. Upon application of pressure and shear force to the methanol treated film, this film fractured and rolled on the skin. With continuous pressure and shear force, the fractured film bits eventually rubbed into the skin. The FTIR spectra shows no difference in the beta sheet content between these two films, however, there is a slight decrease in relative ratio of β-sheet content to glycerin. This suggests that the methanol displaces the glycerin binding to the silk protein and enables more inter-molecular entanglement. Higher inter-molecular entanglement explains the difference in film texture.

### Example 13: FTIR Analysis of the Extrudate and Non-Extrudate Silk Compositions

FTIR analysis was used to investigate the properties of the recombinant spider in the following conditions:
glycerin: 100% glycerin sample
powder: 100% powder sample
powder+glycerin: powder was suspended in glycerin at 25% wt powder, 75% wt glycerin)
powder+glycerin>methanol annealed: powder was suspended in glycerin at 25% wt powder, 75% wt glycerin) and then submerged in methanol for three hours. After three hours the methanol was allowed to dry off
extrudate: 25% wt powder, 75% wt glycerin were mixed and processed through a twin screw extruder at 90 deg C at 250 rpm for 30 minutes
extrudate>resuspended dried: The extrudate material was resuspended in water at 5× dilution by gentle mixing at room temperature. The resuspended extrudate was then dried overnight at ambient temperature and humidity
extrudate>methanol annealed: The extrudate material was submerged in methanol for three hours. After three hours the methanol was allowed to dry off
extrudate>resuspended dried>methanol annealed: The extrudate-resuspended material was submerged in methanol for three hours. After three hours the methanol was allowed to dry off

The FTIR spectra of each were analyzed for β-sheet content and reported as the relative amount of beta sheet content at 1620-1625 cm⁻¹ to total protein content at 1637-1700 cm⁻¹.

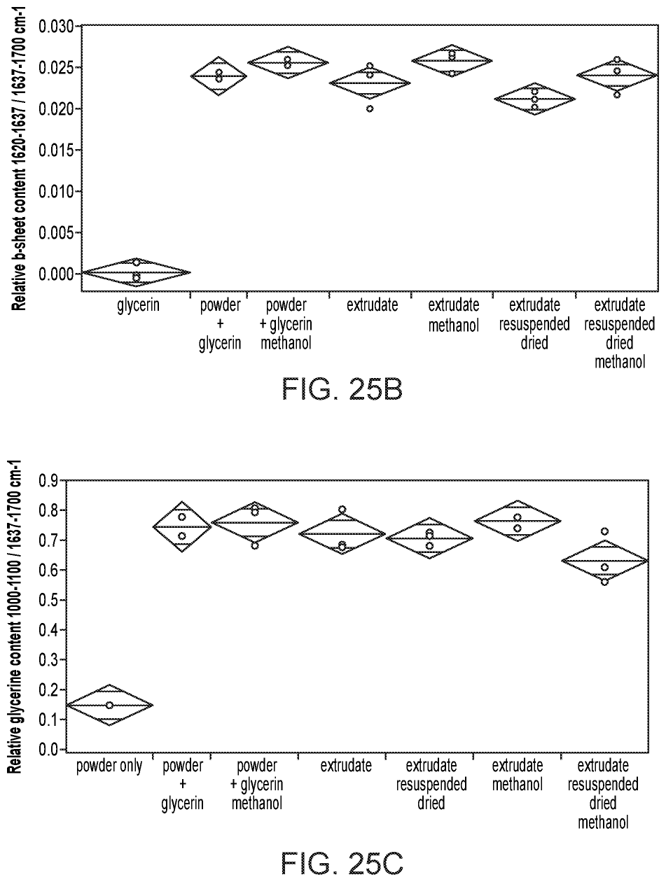

The spectra are shown in FIG. 25A and the quantitation of the relative beta-sheet content is shown in FIG. 25B, including statistical analysis. For the statistical analysis, the top and bottom of the green diamonds represents the 95% confidence interval. The diamond overlap marks appear as lines above and below the group mean and are computed as group mean±(\sqrt{2})/2×CI/2. Overlap marks in one diamond that are closer to the mean of another diamond than that diamond's overlap marks indicate that those two groups are not different at the given confidence level.

In this analysis, the extrudate samples (extrudate, extrudate>resuspended dried, and extrudate>resuspended dried>methanol annealed) are not statistically different than the powder+glycerin sample. This indicates that the process by which powder is turned into extrudate does not affect the beta sheet motif. Rather some other mechanism is needed to explain the phase change between powder and extrudate. This is further underscored by comparing the methanol treated samples. Methanol is a common coagulant for silk-transitioning silk crystalline regions from amorphous configuration to beta-sheet configuration. No difference in beta-sheet content was measured between untreated and methanol treated samples, which further underscores that the mechanism by which the extrusion process transforms powder into extrudate is not governed by beta sheet disruption. While the FTIR spectra show no difference in the beta sheet content between these two films, there is a slight decrease in relative ratio of amino acid content (i.e. amide I band) to glycerin (FIG. 25C).

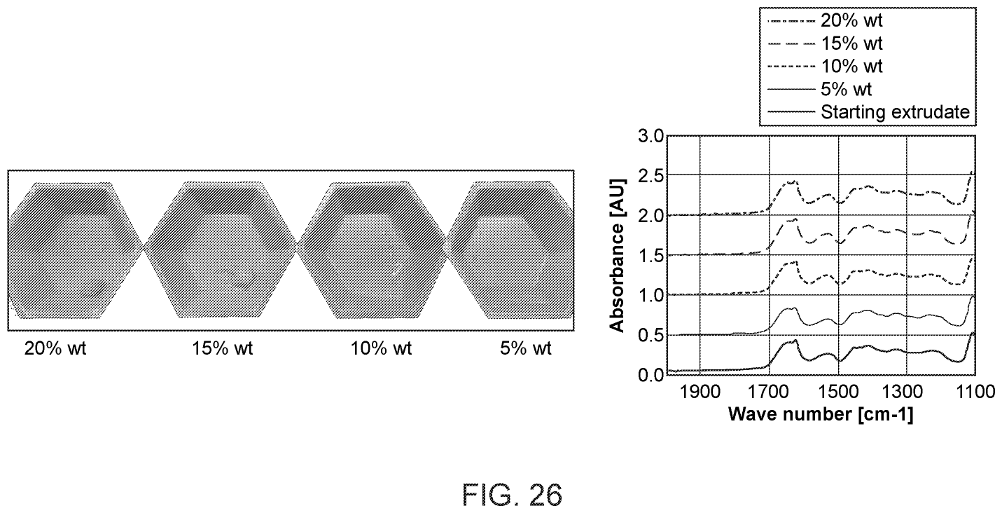

FTIR analysis was also used to investigate the properties of the recombinant spider silk extrudate concentrations in aqueous suspensions after drying to determine whether the amount of water in the aqueous suspended extrudate affected solubility. A spider silk extrudate of 25% recombinant spider silk polypeptide powder and 75% glycerol was suspended in various aqueous solutions to achieve a final amount by weight of recombinant spider silk polypeptide of 5%, 10%, 15% and 20%. The aqueous suspended extrudate was then dried and the FTIR was assessed using the method described above. FIG. 26 depicts the viscosity of the dried down aqueous suspended extrudate and the FTIR peaks. As shown in FIG. 26, notable differences in the viscosity of the dried down aqueous suspended extrudate were observed. However, the FTIR peaks corresponding to beta sheet content were similar across the different aqueous suspended extrudates indicating that the amount of beta sheet formation did not vary with the amount of water in the aqueous suspended extrudate.

### Example 14: Recombinant Silk Colloid Suspension in Extrudate Supernatant

The intention of this example is to quantitatively describe the material properties of the invention and how they are different from the recombinant silk in powder form. When suspending extrudate in an aqueous solvent, the extrudate becomes a colloidal suspension, as determined by particle sizing. This is completely distinct from powder, which when suspended in an aqueous solvent does not significantly partition into the aqueous phase, as evidenced by size exclusion chromatography (SEC).

A colloidal suspension of recombinant silk was prepared by mixing an extrudate in water and centrifuging the mixture to generate a supernatant comprising the colloidal suspension. The protein content in the extrudate supernatant was analyzed by size exclusion chromatography (SEC) and compared with protein content in the silk powder, silk powder supernatant, and extrudate. The size distribution of particles in the colloidal suspension were measured and compared with a 200 nm size standard, a glycerin control, and silk solubilized using LiBr. Details of preparation of the samples and assay results are provided below.

Silk Extrudate Supernatant

Extrudate supernatant was prepared by suspending extrudate (75% glycerin and 25% silk) in water at 20% wt (15% glycerin and 5% silk) with alternate hand shaking and vortexing for —5 min until the solid completely dissipated. This mixture was incubated at RT for 30 mins. The mixture was centrifuged at 16,000 RCF, 30 mins to remove the solids. The supernatant was collected and referred to as the extrudate supernatant.

Silk Powder Supernatant

Powder supernatant was prepared by suspending silk powder as prepared in Example 1 in water at 5% wt and incubating for 30 mins at RT. The mixture was centrifuged at 16,000 RCF, 30 mins to remove the silk powder solids. The supernatant was collected and referred to as the powder supernatant.

LiBr Silk Solubilization

In order to provide a highly solubilized silk sample as a control, 18B silk powder was suspended in 9.3 M LiBr aqueous solution at 1 g of powder to 4 mL of LiBr solution. This solution was incubated at 60° C. for four hours. The dissolved silk was loaded into a dialysis cassette with a 3,500 MW cutoff and dialyzed against 4 L DI water for 48 hours with 6 water changes. The dialyzed silk solution was then centrifuged at 16,000 RCF, 30 mins to remove any precipitated silk. The remaining highly solubilized silk sample was then assayed for particle sizing as provided below.

Protein Profiles

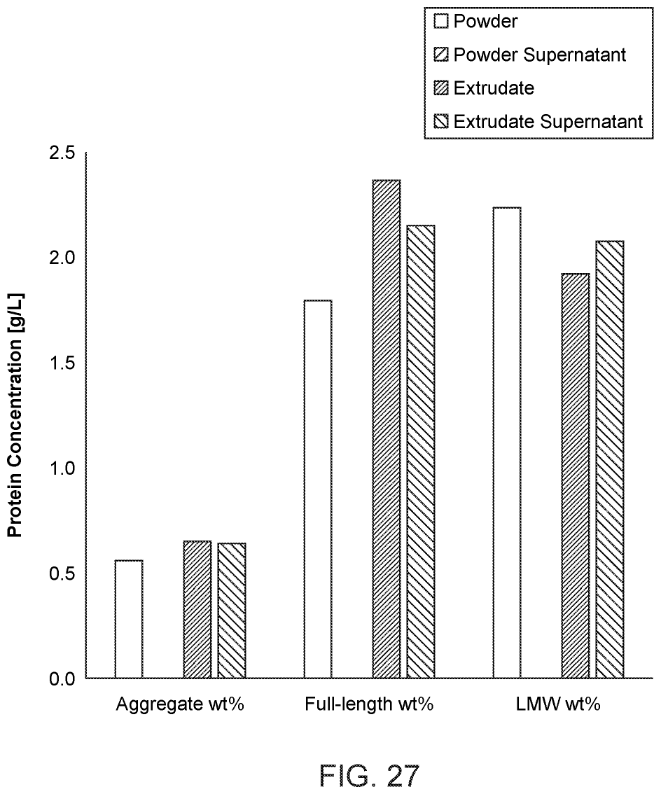

Protein profiles of the silk powder, silk powder supernatant, silk extrudate, and silk extrudate supernatant were measured by SEC. The results are shown in FIG. 27 and provided in Table 16. The powder, extrudate, and extrudate supernatant samples have similar protein profiles. Thus, the silk material that goes into the extrudate supernatant fraction is similar in protein composition as the powder and the extrudate. In other words, there is not a specific aggregate, full-length molecule, or low molecular weight (LMW) fraction that preferentially goes into the extrudate supernatant. Also, the powder supernatant contains undetectable levels of protein, indicating that no protein from the powder solubilizes into the supernatant. Moreover, the protein weight percent content in the extrudate supernatant was 5%, which is the same as the extrudate and the powder, indicating that portion of silk that forms the colloid is not diminished compared to the starting mixture concentration.

TABLE 16
Protein profiles of silk compositions as measured by SEC

| | Aggregate | Full-length | | |
|---|---|---|---|---|
| | wt % | wt % | LMW wt % | Total wt % |
| Powder | 0.56 | 1.795 | 2.235 | 4.59 |
| Powder | 0.00 | 0 | 0 | 0.00 |
| Supernatant | | | | |
| Extrudate | 0.65 | 2.365 | 1.92 | 4.94 |
| Supernatant | 0.64 | 2.15 | 2.075 | 4.87 |

Particle Sizing

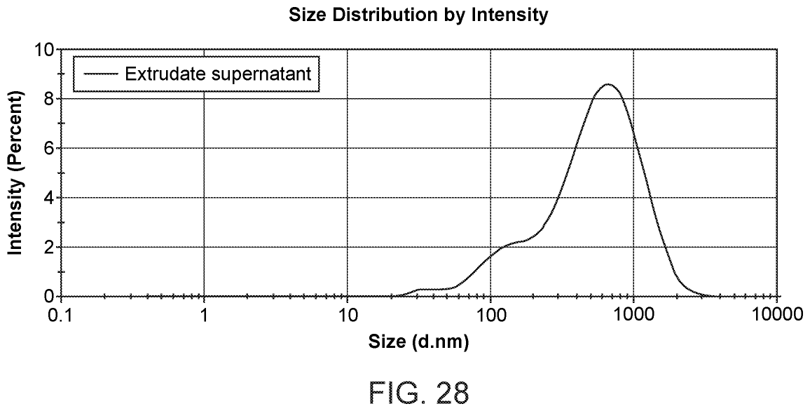

Particle sizing was performed in a Malvern instrument Zetasizer Nano. A polystyrene polymer 200 nm standard was dissolved 250× in water. All samples were diluted in DI water 250× from starting solution (silk starting content was 5% wt, glycerin starting content was 15% wt). Samples were run in single and measured in triplicate. The data reported is the z-average in units of nanometers, accompanied by the polydispersity index (PdI) (FIG. 28 and Table 17). Polydispersity index values closer to zero mean particle sizes are of a single population and values closer to 1 mean particle sizes are of a multitude of populations.

Since SEC does not distinguish between solubilized and unsolubilized silk, particle sizing was used to elucidate the nature of the molecular assembly in the extrudate supernatant (i.e. to determine whether the molecules aggregate into particles and the size of the particles)

As expected, the glycerin and LiBr controls exhibited no peaks. These were fully soluble solutions and should not exhibit peaks. The extrudate supernatant exhibited two peaks and a shoulder region (FIG. 28). The peak values correspond to 38 nm and 642 nm diameter, with a shoulder around 150 nm diameter. Given that colloids are defined as “a mixture that has particles ranging between 1 and 1000 nanometers in diameter, yet are still able to remain evenly distributed throughout the solution,” this data indicates that the resuspended extrudate in water formed a colloidal phase in addition to an undissolved gel phase.

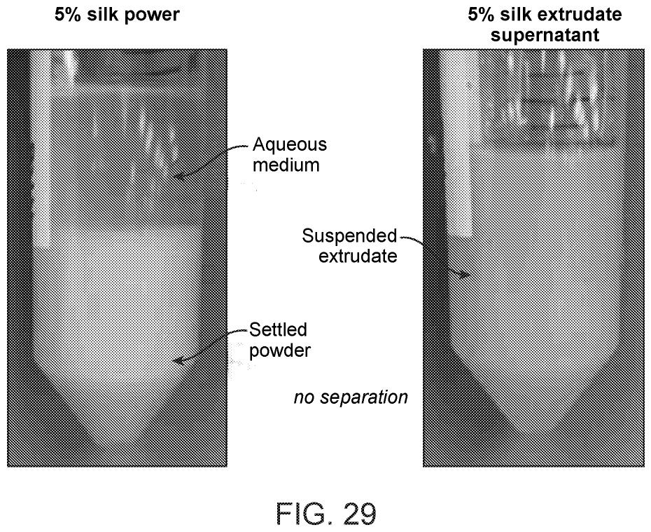

This results is further supported by visual inspection of the powder and extrudate mixtures (FIG. 29). The 5% silk powder mixture settled after 24 hours of incubation at 4° C. The 5% silk extrudate supernatant (i.e. the gel phase has been centrifuged out, this mixture was 5% wt silk and 15% wt glycerin), did not settle after 30 days of incubation at 4° C.

TABLE 17
Particle size distributions in silk solutions
as measured by a Zetasizer Nano

| | | Average Z- | |
|---|---|---|---|
| Sample Name | n | Ave (d · nm) | Average PdI |
| 200 nm Size | 3 | 225.4 | 0.031 |
| Standard | | | |
| Glycerin Control | 1 | 0 | 0 |
| LiBr Control | 3 | 0 | 0 |
| 18B Extrudate | 3 | 350.4 | 0.503 |
| Supernatant | | | |

### Example 15: Barrier Repair Assay

Six subjects (Age 38.2 Years ; 5 females-1 Male) were tested for skin barrier repair using 5% silk protein extrudate mixtures (the extrudate going into the mixture was 75% glycerin and 25% silk protein, the resulting mixture was 15% glycerin and 5% silk protein). The volar forearm was delineated into three sections. Trans epidermal water loss (TEWL) was measured by a vapometer. The skin was tape stripped with packing tape until the TEWL values reached between 20 and 25. Product was applied and TEWL was measured again at 30 minutes and 2 hours. A vehicle control (15% glycerin in water) and an untreatment site were included in the study.

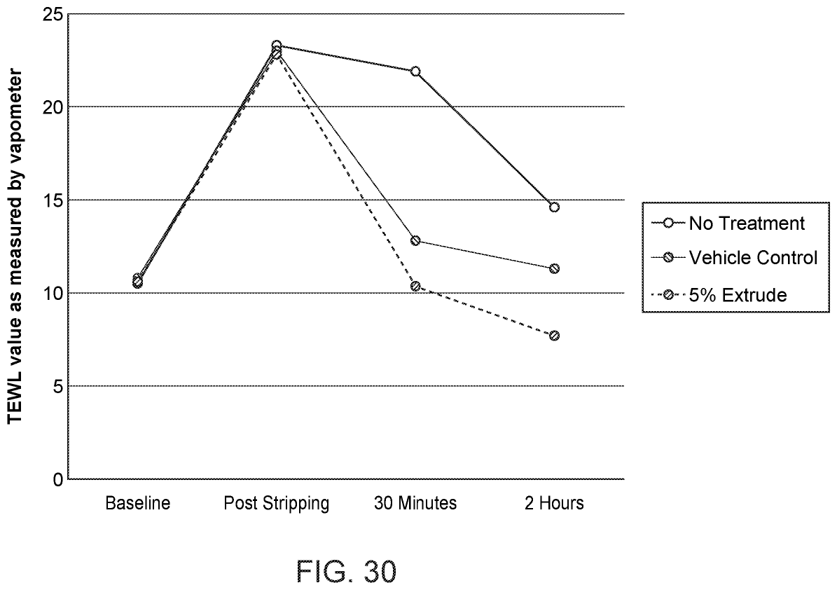

The 5% extrudate sample exhibited the most rapid repair compared to the controls as well as returning to baseline faster than the controls (FIG. 30).

What is claimed is:

1. A method of making a silk-based emulsion, comprising:
mixing a composition comprising a recombinant spider silk polypeptide powder and glycerol by applying pressure and shear force to the composition, thereby transforming the composition to an extrudate;
suspending at least a portion of said extrudate in an aqueous solvent to form an aqueous extrudate suspension; and
mixing said aqueous extrudate suspension into an emulsion to form said silk-based emulsion.

2. The method of claim 1, wherein said extrudate is substantially homogenous.

3. The method of claim 1, wherein said silk-based emulsion is a cosmetic or skincare formulation.

4. A method of making a silk-based solid or gel, comprising:
mixing a composition comprising a recombinant spider silk polypeptide powder and glycerol by applying pressure and shear force to the composition, thereby transforming the composition to an extrudate;
suspending said extrudate in an aqueous solvent to form an aqueous extrudate suspension; and
drying said aqueous extrudate suspension to form a silk-based solid or gel.

5. The method of claim 4, further comprising coagulating said aqueous extrudate suspension to form aggregated silk in said suspension.

6. The method of claim 4, wherein said silk-based solid or gel is a film.

7. The method of claim 6, wherein said silk-based solid is a cosmetic or skincare formulation.

8. A method of making a silk-based formulation, comprising:
providing a composition comprising a silk protein and a plasticizer;
applying pressure and shear force to the composition, thereby transforming the composition to an extrudate; and
suspending said extrudate in an aqueous solvent to form an aqueous extrudate suspension.

9. The method of claim 8, further comprising drying said aqueous extrudate suspension to form a silk-based solid or gel.

10. The method of claim 8, further comprising mixing said aqueous extrudate suspension into an emulsion to form said silk-based emulsion.

11. The method of claim 10, further comprising drying said silk-based emulsion to form a silk-based solid or gel.

12. The method of claim 9 or 11, further comprising adding a coagulant or an additive to said silk-based solid or gel to form a more solid gel or solid.

13. The method of any one of claims 8-12, further comprising coagulating said aqueous extrudate suspension to form aggregated silk in said suspension.

14. The method of claim 8, wherein said aqueous extrudate suspension comprises a gel phase, a colloidal phase, and a solution phase.

15. The method of claim 14, further comprising separating said gel phase, said colloidal phase, or said solution phase from said aqueous extrudate suspension.

16. The method of claim 15, further comprising drying said gel phase, said colloidal phase, or said solution phase to form a silk-based solid or gel.

17. The method of claim 14, further comprising separating a mixture of said colloidal phase and said solution phase from said aqueous extrudate suspension.

18. The method of claim 17, further comprising drying said mixture of said colloidal phase and said solution phase to form a silk-based solid or gel.

19. The method of claim 14, wherein the silk is recombinant spider silk.

20. The method of claim 19, wherein said recombinant spider silk comprises full length proteins.

21. The method of claim 9 or 11, wherein the silk-based solid or gel is a skincare or cosmetic formulation.

22. The method of claim 10, wherein the silk-based emulsion is a skincare or cosmetic formulation.

23. The method of claim 8, wherein said plasticizer is glycerin.

24. The method of claim 8, wherein the extrudate is in a flowable state.

25. The method of claim 8, wherein said aqueous solution is water.

26. The method of claim 12, wherein said coagulant is methanol.

27. The method of claim 9 or 11, wherein said silk-based solid or gel is non-toxic.

28. The method of claim 10, wherein the silk-based emulsion is non-toxic.

29. The method of claim 8, wherein said applied shear force is at least 1.5 Newton meters.

30. The method of claim 8, wherein said applied pressure is at least 1 MPa.

31. The method of claim 8, further comprising agitating said aqueous extrudate suspension.

32. The method of claim 8, further comprising applying heat to said aqueous extrudate suspension.

33. The method of claim 9 or 11, wherein the silk-based solid or gel is a film.

34. The method of claim 33, wherein said film disperses upon contact with skin or water or gentle rubbing.

35. The method of claim 33, wherein said film disperses into a liquid at a temperature of less than 37° C., but more than 23° C.

36. A method of making a silk-based gel, colloid or solution, comprising:
mixing a composition comprising a silk protein and a plasticizer by applying pressure and shear force to the composition, thereby transforming the composition to an extrudate;
suspending said extrudate in an aqueous solvent to form an aqueous suspended extrudate;
heating and/or agitating said aqueous suspended extrudate to form a gel phase, a colloidal phase, and solution phase; and
separating said phases to generate a silk-based gel, colloid or solution.

37. A composition comprising:
an extrudate comprising a recombinant silk protein and a plasticizer, wherein said extrudate is suspended in an aqueous solution.

38. The composition of claim 37, wherein said extrudate is evenly dispersed as particles in said aqueous solution.

39. The composition of claim 38, wherein said particles in said aqueous solution have a polydispersity index from 0.1 to 0.9.

40. The composition of claim 38, wherein said particles in said aqueous solution have a z-average of about 600 to 1,000 nm.

41. The composition of claim 37, wherein said extrudate suspended in said aqueous solution forms a colloid solution.

42. The composition of claim 37, wherein said composition further comprises a coagulant.

43. The composition of claim 37, wherein said plasticizer is glycerol.

44. The composition of claim 37, wherein said composition is a film.

45. The composition of claim 37, wherein said film is stable at room temperature and disperses upon contact with skin or water.

46. The composition of claim 37, wherein said recombinant silk protein is substantially full length protein.

47. The composition of claim 37, wherein said recombinant silk protein is not substantially aggregated in said composition.

48. The composition of claim 37, wherein said recombinant silk protein has a decreased, similar, or increased crystallinity as compared to the recombinant silk protein in powder form.

49. A spider silk cosmetic or skincare product comprising an extrudate comprising a silk protein and a plasticizer, wherein said extrudate is dispersed in an aqueous solvent or coagulant in a gel, colloid, or solution phase.

50. The composition of claim 49, wherein said extrudate is dispersed in said aqueous solvent and said coagulant.

51. The composition of claim 49, wherein said spider silk cosmetic or skincare product is an emulsion or an aqueous solution.

52. A spider silk cosmetic or skincare product comprising a solid or semi-solid, wherein said solid or semi-solid comprises dispersed non-aggregated recombinant silk protein and a plasticizer.

53. The composition of claim 52, wherein said solid or semi-solid dissolves upon contact with skin.

54. The composition of claim 53, wherein said solid or semi-solid is a film.


SEQUENCE LISTING (38 sequences)

SEQ ID NO: 1 - protein, 945 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 315 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 3 - protein, 1600 aa, Artificial Sequence
  Description of Artificial Sequence Synthetic polypeptide
  MISC_FEATURE (7)..(11): This region may encompass “SGGQQ,” “GAGQQ,” “GQGPY,” “AGQQ” or “SQ,” wherein some positions may be absent
  MISC_FEATURE (15)..(19): This region may encompass “SGGQQ,” “GAGQQ,” “GQGPY,” “AGQQ” or “SQ,” wherein some positions may be absent
  MISC_FEATURE (23)..(27): This region may encompass “SGGQQ,” “GAGQQ,” “GQGPY,” “AGQQ” or “SQ,” wherein some positions may be absent
  MISC_FEATURE (31)..(35): This region may encompass “SGGQQ,” “GAGQQ,” “GQGPY,” “AGQQ” or “SQ,” wherein some positions may be absent
  MISC_FEATURE (39)..(43): This region may encompass “SGGQQ,” “GAGQQ,” “GQGPY,” “AGQQ” or “SQ,” wherein some positions may be absent
  MISC_FEATURE (47)..(51): This region may encompass “SGGQQ,” “GAGQQ,” “GQGPY,” “AGQQ” or “SQ,” wherein some positions may be absent
  MISC_FEATURE (55)..(59): This region may encompass “SGGQQ,” “GAGQQ,” “GQGPY,” “AGQQ” or “SQ,” wherein some positions may be absent
  MISC_FEATURE (63)..(67): This region may encompass “SGGQQ,” “GAGQQ,” “GQGPY,” “AGQQ” or “SQ,” wherein some positions may be absent
  MISC_FEATURE (4)..(67): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (71)..(80): This region may encompass 6-10 residues
  MISC_FEATURE (87)..(91): This region may encompass “SGGQQ,” “GAGQQ,” “GQGPY,” “AGQQ” or “SQ,” wherein some positions may be absent
  MISC_FEATURE (95)..(99): This region may encompass “SGGQQ,” “GAGQQ,” “GQGPY,” “AGQQ” or “SQ,” wherein some positions may be absent
  MISC_FEATURE (103)..(107): This region may encompass “SGGQQ,” “GAGQQ,” “GQGPY,” “AGQQ” or “SQ,” wherein some positions may be absent
  MISC_FEATURE (111)..(115): This region may encompass “SGGQQ,” “GAGQQ,” “GQGPY,” “AGQQ” or “SQ,” wherein some positions may be absent
  MISC_FEATURE (119)..(123): This region may encompass “SGGQQ,” “GAGQQ,” “GQGPY,” “AGQQ” or “SQ,” wherein some positions may be absent
  MISC_FEATURE (127)..(131): This region may encompass “SGGQQ,” “GAGQQ,” “GQGPY,” “AGQQ” or “SQ,” wherein some positions may be absent
  MISC_FEATURE (135)..(139): This region may encompass “SGGQQ,” “GAGQQ,” “GQGPY,” “AGQQ” or “SQ,” wherein some positions may be absent
  MISC_FEATURE (143)..(147): This region may encompass “SGGQQ,” “GAGQQ,” “GQGPY,” “AGQQ” or “SQ,” wherein some positions may be absent
  MISC_FEATURE (84)..(147): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (151)..(160): This region may encompass 6-10 residues
  MISC_FEATURE (167)..(171): This region may encompass “SGGQQ,” “GAGQQ,” “GQGPY,” “AGQQ” or “SQ,” wherein some positions may be absent
  MISC_FEATURE (175)..(179): This region may encompass “SGGQQ,” “GAGQQ,” “GQGPY,” “AGQQ” or “SQ,” wherein some positions may be absent
  MISC_FEATURE (183)..(187): This region may encompass “SGGQQ,” “GAGQQ,” “GQGPY,” “AGQQ” or “SQ,” wherein some positions may be absent
  MISC_FEATURE (191)..(195): This region may encompass “SGGQQ,” “GAGQQ,” “GQGPY,” “AGQQ” or “SQ,” wherein some positions may be absent
  MISC_FEATURE (199)..(203): This region may encompass “SGGQQ,” “GAGQQ,” “GQGPY,” “AGQQ” or “SQ,” wherein some positions may be absent
  MISC_FEATURE (207)..(211): This region may encompass “SGGQQ,” “GAGQQ,” “GQGPY,” “AGQQ” or “SQ,” wherein some positions may be absent
  MISC_FEATURE (215)..(219): This region may encompass “SGGQQ,” “GAGQQ,” “GQGPY,” “AGQQ” or “SQ,” wherein some positions may be absent
  MISC_FEATURE (223)..(227): This region may encompass “SGGQQ,” “GAGQQ,” “GQGPY,” “AGQQ” or “SQ,” wherein some positions may be absent
  MISC_FEATURE (164)..(227): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (231)..(240): This region may encompass 6-10 residues
  MISC_FEATURE (247)..(251): This region may encompass “SGGQQ,” “GAGQQ,” “GQGPY,” “AGQQ” or “SQ,” wherein some positions may be absent
  MISC_FEATURE (255)..(259): This region may encompass “SGGQQ,” “GAGQQ,” “GQGPY,” “AGQQ” or “SQ,” wherein some positions may be absent
  MISC_FEATURE (263)..(267): This region may encompass “SGGQQ,” “GAGQQ,” “GQGPY,” “AGQQ” or “SQ,” wherein some positions may be absent
  MISC_FEATURE (271)..(275): This region may encompass “SGGQQ,” “GAGQQ,” “GQGPY,” “AGQQ” or “SQ,” wherein some positions may be absent
  MISC_FEATURE (279)..(283): This region may encompass “SGGQQ,” “GAGQQ,” “GQGPY,” “AGQQ” or “SQ,” wherein some positions may be absent
  MISC_FEATURE (287)..(291): This region may encompass “SGGQQ,” “GAGQQ,” “GQGPY,” “AGQQ” or “SQ,” wherein some positions may be absent
  MISC_FEATURE (295)..(299): This region may encompass “SGGQQ,” “GAGQQ,” “GQGPY,” “AGQQ” or “SQ,” wherein some positions may be absent
  MISC_FEATURE (303)..(307): This region may encompass “SGGQQ,” “GAGQQ,” “GQGPY,” “AGQQ” or “SQ,” wherein some positions may be absent
  MISC_FEATURE (244)..(307): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (311)..(320): This region may encompass 6-10 residues
  MISC_FEATURE (327)..(331): This region may encompass “SGGQQ,” “GAGQQ,” “GQGPY,” “AGQQ” or “SQ,” wherein some positions may be absent
  MISC_FEATURE (335)..(339): This region may encompass “SGGQQ,” “GAGQQ,” “GQGPY,” “AGQQ” or “SQ,” wherein some positions may be absent
  MISC_FEATURE (343)..(347): This region may encompass “SGGQQ,” “GAGQQ,” “GQGPY,” “AGQQ” or “SQ,” wherein some positions may be absent
  MISC_FEATURE (351)..(355): This region may encompass “SGGQQ,” “GAGQQ,” “GQGPY,” “AGQQ” or “SQ,” wherein some positions may be absent
  MISC_FEATURE (359)..(363): This region may encompass “SGGQQ,” “GAGQQ,” “GQGPY,” “AGQQ” or “SQ,” wherein some positions may be absent
  MISC_FEATURE (367)..(371): This region may encompass “SGGQQ,” “GAGQQ,” “GQGPY,” “AGQQ” or “SQ,” wherein some positions may be absent
  MISC_FEATURE (375)..(379): This region may encompass “SGGQQ,” “GAGQQ,” “GQGPY,” “AGQQ” or “SQ,” wherein some positions may be absent
  MISC_FEATURE (383)..(387): This region may encompass “SGGQQ,” “GAGQQ,” “GQGPY,” “AGQQ” or “SQ,” wherein some positions may be absent
  MISC_FEATURE (324)..(387): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (391)..(400): This region may encompass 6-10 residues
  MISC_FEATURE (407)..(411): This region may encompass “SGGQQ,” “GAGQQ,” “GQGPY,” “AGQQ” or “SQ,” wherein some positions may be absent
  MISC_FEATURE (415)..(419): This region may encompass “SGGQQ,” “GAGQQ,” “GQGPY,” “AGQQ” or “SQ,” wherein some positions may be absent
  MISC_FEATURE (423)..(427): This region may encompass “SGGQQ,” “GAGQQ,” “GQGPY,” “AGQQ” or “SQ,” wherein some positions may be absent
  MISC_FEATURE (431)..(435): This region may encompass “SGGQQ,” “GAGQQ,” “GQGPY,” “AGQQ” or “SQ,” wherein some positions may be absent
  MISC_FEATURE (439)..(443): This region may encompass “SGGQQ,” “GAGQQ,” “GQGPY,” “AGQQ” or “SQ,” wherein some positions may be absent
  MISC_FEATURE (447)..(451): This region may encompass “SGGQQ,” “GAGQQ,” “GQGPY,” “AGQQ” or “SQ,” wherein some positions may be absent
  MISC_FEATURE (455)..(459): This region may encompass “SGGQQ,” “GAGQQ,” “GQGPY,” “AGQQ” or “SQ,” wherein some positions may be absent
  MISC_FEATURE (463)..(467): This region may encompass “SGGQQ,” “GAGQQ,” “GQGPY,” “AGQQ” or “SQ,” wherein some positions may be absent
  MISC_FEATURE (404)..(467): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (471)..(480): This region may encompass 6-10 residues
  MISC_FEATURE (487)..(491): This region may encompass “SGGQQ,” “GAGQQ,” “GQGPY,” “AGQQ” or “SQ,” wherein some positions may be absent
  MISC_FEATURE (495)..(499): This region may encompass “SGGQQ,” “GAGQQ,” “GQGPY,” “AGQQ” or “SQ,” wherein some positions may be absent
  MISC_FEATURE (503)..(507): This region may encompass “SGGQQ,” “GAGQQ,” “GQGPY,” “AGQQ” or “SQ,” wherein some positions may be absent
  MISC_FEATURE (511)..(515): This region may encompass “SGGQQ,” “GAGQQ,” “GQGPY,” “AGQQ” or “SQ,” wherein some positions may be absent
  MISC_FEATURE (519)..(523): This region may encompass “SGGQQ,” “GAGQQ,” “GQGPY,” “AGQQ” or “SQ,” wherein some positions may be absent
  MISC_FEATURE (527)..(531): This region may encompass “SGGQQ,” “GAGQQ,” “GQGPY,” “AGQQ” or “SQ,” wherein some positions may be absent
  MISC_FEATURE (535)..(539): This region may encompass “SGGQQ,” “GAGQQ,” “GQGPY,” “AGQQ” or “SQ,” wherein some positions may be absent
  MISC_FEATURE (543)..(547): This region may encompass “SGGQQ,” “GAGQQ,” “GQGPY,” “AGQQ” or “SQ,” wherein some positions may be absent
  MISC_FEATURE (484)..(547): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (551)..(560): This region may encompass 6-10 residues
  MISC_FEATURE (567)..(571): This region may encompass “SGGQQ,” “GAGQQ,” “GQGPY,” “AGQQ” or “SQ,” wherein some positions may be absent
  MISC_FEATURE (575)..(579): This region may encompass “SGGQQ,” “GAGQQ,” “GQGPY,” “AGQQ” or “SQ,” wherein some positions may be absent
  MISC_FEATURE (583)..(587): This region may encompass “SGGQQ,” “GAGQQ,” “GQGPY,” “AGQQ” or “SQ,” wherein some positions may be absent
  MISC_FEATURE (591)..(595): This region may encompass “SGGQQ,” “GAGQQ,” “GQGPY,” “AGQQ” or “SQ,” wherein some positions may be absent
  MISC_FEATURE (599)..(603): This region may encompass “SGGQQ,” “GAGQQ,” “GQGPY,” “AGQQ” or “SQ,” wherein some positions may be absent
  MISC_FEATURE (607)..(611): This region may encompass “SGGQQ,” “GAGQQ,” “GQGPY,” “AGQQ” or “SQ,” wherein some positions may be absent
  MISC_FEATURE (615)..(619): This region may encompass “SGGQQ,” “GAGQQ,” “GQGPY,” “AGQQ” or “SQ,” wherein some positions may be absent
  MISC_FEATURE (623)..(627): This region may encompass “SGGQQ,” “GAGQQ,” “GQGPY,” “AGQQ” or “SQ,” wherein some positions may be absent
  MISC_FEATURE (564)..(627): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (631)..(640): This region may encompass 6-10 residues
  MISC_FEATURE (647)..(651): This region may encompass “SGGQQ,” “GAGQQ,” “GQGPY,” “AGQQ” or “SQ,” wherein some positions may be absent
  MISC_FEATURE (655)..(659): This region may encompass “SGGQQ,” “GAGQQ,” “GQGPY,” “AGQQ” or “SQ,” wherein some positions may be absent
  MISC_FEATURE (663)..(667): This region may encompass “SGGQQ,” “GAGQQ,” “GQGPY,” “AGQQ” or “SQ,” wherein some positions may be absent
  MISC_FEATURE (671)..(675): This region may encompass “SGGQQ,” “GAGQQ,” “GQGPY,” “AGQQ” or “SQ,” wherein some positions may be absent
  MISC_FEATURE (679)..(683): This region may encompass “SGGQQ,” “GAGQQ,” “GQGPY,” “AGQQ” or “SQ,” wherein some positions may be absent
  MISC_FEATURE (687)..(691): This region may encompass “SGGQQ,” “GAGQQ,” “GQGPY,” “AGQQ” or “SQ,” wherein some positions may be absent
  MISC_FEATURE (695)..(699): This region may encompass “SGGQQ,” “GAGQQ,” “GQGPY,” “AGQQ” or “SQ,” wherein some positions may be absent
  MISC_FEATURE (703)..(707): This region may encompass “SGGQQ,” “GAGQQ,” “GQGPY,” “AGQQ” or “SQ,” wherein some positions may be absent
  MISC_FEATURE (644)..(707): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (711)..(720): This region may encompass 6-10 residues
  MISC_FEATURE (727)..(731): This region may encompass “SGGQQ,” “GAGQQ,” “GQGPY,” “AGQQ” or “SQ,” wherein some positions may be absent
  MISC_FEATURE (735)..(739): This region may encompass “SGGQQ,” “GAGQQ,” “GQGPY,” “AGQQ” or “SQ,” wherein some positions may be absent
  MISC_FEATURE (743)..(747): This region may encompass “SGGQQ,” “GAGQQ,” “GQGPY,” “AGQQ” or “SQ,” wherein some positions may be absent
  MISC_FEATURE (751)..(755): This region may encompass “SGGQQ,” “GAGQQ,” “GQGPY,” “AGQQ” or “SQ,” wherein some positions may be absent
  MISC_FEATURE (759)..(763): This region may encompass “SGGQQ,” “GAGQQ,” “GQGPY,” “AGQQ” or “SQ,” wherein some positions may be absent
  MISC_FEATURE (767)..(771): This region may encompass “SGGQQ,” “GAGQQ,” “GQGPY,” “AGQQ” or “SQ,” wherein some positions may be absent
  MISC_FEATURE (775)..(779): This region may encompass “SGGQQ,” “GAGQQ,” “GQGPY,” “AGQQ” or “SQ,” wherein some positions may be absent
  MISC_FEATURE (783)..(787): This region may encompass “SGGQQ,” “GAGQQ,” “GQGPY,” “AGQQ” or “SQ,” wherein some positions may be absent
  MISC_FEATURE (724)..(787): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (791)..(800): This region may encompass 6-10 residues
  MISC_FEATURE (807)..(811): This region may encompass “SGGQQ,” “GAGQQ,” “GQGPY,” “AGQQ” or “SQ,” wherein some positions may be absent
  MISC_FEATURE (815)..(819): This region may encompass “SGGQQ,” “GAGQQ,” “GQGPY,” “AGQQ” or “SQ,” wherein some positions may be absent
  MISC_FEATURE (823)..(827): This region may encompass “SGGQQ,” “GAGQQ,” “GQGPY,” “AGQQ” or “SQ,” wherein some positions may be absent
  MISC_FEATURE (831)..(835): This region may encompass “SGGQQ,” “GAGQQ,” “GQGPY,” “AGQQ” or “SQ,” wherein some positions may be absent
  MISC_FEATURE (839)..(843): This region may encompass “SGGQQ,” “GAGQQ,” “GQGPY,” “AGQQ” or “SQ,” wherein some positions may be absent
  MISC_FEATURE (847)..(851): This region may encompass “SGGQQ,” “GAGQQ,” “GQGPY,” “AGQQ” or “SQ,” wherein some positions may be absent
  MISC_FEATURE (855)..(859): This region may encompass “SGGQQ,” “GAGQQ,” “GQGPY,” “AGQQ” or “SQ,” wherein some positions may be absent
  MISC_FEATURE (863)..(867): This region may encompass “SGGQQ,” “GAGQQ,” “GQGPY,” “AGQQ” or “SQ,” wherein some positions may be absent
  MISC_FEATURE (804)..(867): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (871)..(880): This region may encompass 6-10 residues
  MISC_FEATURE (887)..(891): This region may encompass “SGGQQ,” “GAGQQ,” “GQGPY,” “AGQQ” or “SQ,” wherein some positions may be absent
  MISC_FEATURE (895)..(899): This region may encompass “SGGQQ,” “GAGQQ,” “GQGPY,” “AGQQ” or “SQ,” wherein some positions may be absent
  MISC_FEATURE (903)..(907): This region may encompass “SGGQQ,” “GAGQQ,” “GQGPY,” “AGQQ” or “SQ,” wherein some positions may be absent
  MISC_FEATURE (911)..(915): This region may encompass “SGGQQ,” “GAGQQ,” “GQGPY,” “AGQQ” or “SQ,” wherein some positions may be absent
  MISC_FEATURE (919)..(923): This region may encompass “SGGQQ,” “GAGQQ,” “GQGPY,” “AGQQ” or “SQ,” wherein some positions may be absent
  MISC_FEATURE (927)..(931): This region may encompass “SGGQQ,” “GAGQQ,” “GQGPY,” “AGQQ” or “SQ,” wherein some positions may be absent
  MISC_FEATURE (935)..(939): This region may encompass “SGGQQ,” “GAGQQ,” “GQGPY,” “AGQQ” or “SQ,” wherein some positions may be absent
  MISC_FEATURE (943)..(947): This region may encompass “SGGQQ,” “GAGQQ,” “GQGPY,” “AGQQ” or “SQ,” wherein some positions may be absent
  MISC_FEATURE (884)..(947): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (951)..(960): This region may encompass 6-10 residues
  MISC_FEATURE (967)..(971): This region may encompass “SGGQQ,” “GAGQQ,” “GQGPY,” “AGQQ” or “SQ,” wherein some positions may be absent
  MISC_FEATURE (975)..(979): This region may encompass “SGGQQ,” “GAGQQ,” “GQGPY,” “AGQQ” or “SQ,” wherein some positions may be absent
  MISC_FEATURE (983)..(987): This region may encompass “SGGQQ,” “GAGQQ,” “GQGPY,” “AGQQ” or “SQ,” wherein some positions may be absent
  MISC_FEATURE (991)..(995): This region may encompass “SGGQQ,” “GAGQQ,” “GQGPY,” “AGQQ” or “SQ,” wherein some positions may be absent
  MISC_FEATURE (999)..(1003): This region may encompass “SGGQQ,” “GAGQQ,” “GQGPY,” “AGQQ” or “SQ,” wherein some positions may be absent
  MISC_FEATURE (1007)..(1011): This region may encompass “SGGQQ,” “GAGQQ,” “GQGPY,” “AGQQ” or “SQ,” wherein some positions may be absent
  MISC_FEATURE (1015)..(1019): This region may encompass “SGGQQ,” “GAGQQ,” “GQGPY,” “AGQQ” or “SQ,” wherein some positions may be absent
  MISC_FEATURE (1023)..(1027): This region may encompass “SGGQQ,” “GAGQQ,” “GQGPY,” “AGQQ” or “SQ,” wherein some positions may be absent
  MISC_FEATURE (964)..(1027): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1031)..(1040): This region may encompass 6-10 residues
  MISC_FEATURE (1047)..(1051): This region may encompass “SGGQQ,” “GAGQQ,” “GQGPY,” “AGQQ” or “SQ,” wherein some positions may be absent
  MISC_FEATURE (1055)..(1059): This region may encompass “SGGQQ,” “GAGQQ,” “GQGPY,” “AGQQ” or “SQ,” wherein some positions may be absent
  MISC_FEATURE (1063)..(1067): This region may encompass “SGGQQ,” “GAGQQ,” “GQGPY,” “AGQQ” or “SQ,” wherein some positions may be absent
  MISC_FEATURE (1071)..(1075): This region may encompass “SGGQQ,” “GAGQQ,” “GQGPY,” “AGQQ” or “SQ,” wherein some positions may be absent
  MISC_FEATURE (1079)..(1083): This region may encompass “SGGQQ,” “GAGQQ,” “GQGPY,” “AGQQ” or “SQ,” wherein some positions may be absent
  MISC_FEATURE (1087)..(1091): This region may encompass “SGGQQ,” “GAGQQ,” “GQGPY,” “AGQQ” or “SQ,” wherein some positions may be absent
  MISC_FEATURE (1095)..(1099): This region may encompass “SGGQQ,” “GAGQQ,” “GQGPY,” “AGQQ” or “SQ,” wherein some positions may be absent
  MISC_FEATURE (1103)..(1107): This region may encompass “SGGQQ,” “GAGQQ,” “GQGPY,” “AGQQ” or “SQ,” wherein some positions may be absent
  MISC_FEATURE (1044)..(1107): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1111)..(1120): This region may encompass 6-10 residues
  MISC_FEATURE (1127)..(1131): This region may encompass “SGGQQ,” “GAGQQ,” “GQGPY,” “AGQQ” or “SQ,” wherein some positions may be absent
  MISC_FEATURE (1135)..(1139): This region may encompass “SGGQQ,” “GAGQQ,” “GQGPY,” “AGQQ” or “SQ,” wherein some positions may be absent
  MISC_FEATURE (1143)..(1147): This region may encompass “SGGQQ,” “GAGQQ,” “GQGPY,” “AGQQ” or “SQ,” wherein some positions may be absent
  MISC_FEATURE (1151)..(1155): This region may encompass “SGGQQ,” “GAGQQ,” “GQGPY,” “AGQQ” or “SQ,” wherein some positions may be absent
  MISC_FEATURE (1159)..(1163): This region may encompass “SGGQQ,” “GAGQQ,” “GQGPY,” “AGQQ” or “SQ,” wherein some positions may be absent
  MISC_FEATURE (1167)..(1171): This region may encompass “SGGQQ,” “GAGQQ,” “GQGPY,” “AGQQ” or “SQ,” wherein some positions may be absent
  MISC_FEATURE (1175)..(1179): This region may encompass “SGGQQ,” “GAGQQ,” “GQGPY,” “AGQQ” or “SQ,” wherein some positions may be absent
  MISC_FEATURE (1183)..(1187): This region may encompass “SGGQQ,” “GAGQQ,” “GQGPY,” “AGQQ” or “SQ,” wherein some positions may be absent
  MISC_FEATURE (1124)..(1187): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1191)..(1200): This region may encompass 6-10 residues
  MISC_FEATURE (1207)..(1211): This region may encompass “SGGQQ,” “GAGQQ,” “GQGPY,” “AGQQ” or “SQ,” wherein some positions may be absent
  MISC_FEATURE (1215)..(1219): This region may encompass “SGGQQ,” “GAGQQ,” “GQGPY,” “AGQQ” or “SQ,” wherein some positions may be absent
  MISC_FEATURE (1223)..(1227): This region may encompass “SGGQQ,” “GAGQQ,” “GQGPY,” “AGQQ” or “SQ,” wherein some positions may be absent
  MISC_FEATURE (1231)..(1235): This region may encompass “SGGQQ,” “GAGQQ,” “GQGPY,” “AGQQ” or “SQ,” wherein some positions may be absent
  MISC_FEATURE (1239)..(1243): This region may encompass “SGGQQ,” “GAGQQ,” “GQGPY,” “AGQQ” or “SQ,” wherein some positions may be absent
  MISC_FEATURE (1247)..(1251): This region may encompass “SGGQQ,” “GAGQQ,” “GQGPY,” “AGQQ” or “SQ,” wherein some positions may be absent
  MISC_FEATURE (1255)..(1259): This region may encompass “SGGQQ,” “GAGQQ,” “GQGPY,” “AGQQ” or “SQ,” wherein some positions may be absent
  MISC_FEATURE (1263)..(1267): This region may encompass “SGGQQ,” “GAGQQ,” “GQGPY,” “AGQQ” or “SQ,” wherein some positions may be absent
  MISC_FEATURE (1204)..(1267): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1271)..(1280): This region may encompass 6-10 residues
  MISC_FEATURE (1287)..(1291): This region may encompass “SGGQQ,” “GAGQQ,” “GQGPY,” “AGQQ” or “SQ,” wherein some positions may be absent
  MISC_FEATURE (1295)..(1299): This region may encompass “SGGQQ,” “GAGQQ,” “GQGPY,” “AGQQ” or “SQ,” wherein some positions may be absent
  MISC_FEATURE (1303)..(1307): This region may encompass “SGGQQ,” “GAGQQ,” “GQGPY,” “AGQQ” or “SQ,” wherein some positions may be absent
  MISC_FEATURE (1311)..(1315): This region may encompass “SGGQQ,” “GAGQQ,” “GQGPY,” “AGQQ” or “SQ,” wherein some positions may be absent
  MISC_FEATURE (1319)..(1323): This region may encompass “SGGQQ,” “GAGQQ,” “GQGPY,” “AGQQ” or “SQ,” wherein some positions may be absent
  MISC_FEATURE (1327)..(1331): This region may encompass “SGGQQ,” “GAGQQ,” “GQGPY,” “AGQQ” or “SQ,” wherein some positions may be absent
  MISC_FEATURE (1335)..(1339): This region may encompass “SGGQQ,” “GAGQQ,” “GQGPY,” “AGQQ” or “SQ,” wherein some positions may be absent
  MISC_FEATURE (1343)..(1347): This region may encompass “SGGQQ,” “GAGQQ,” “GQGPY,” “AGQQ” or “SQ,” wherein some positions may be absent
  MISC_FEATURE (1284)..(1347): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1351)..(1360): This region may encompass 6-10 residues
  MISC_FEATURE (1367)..(1371): This region may encompass “SGGQQ,” “GAGQQ,” “GQGPY,” “AGQQ” or “SQ,” wherein some positions may be absent
  MISC_FEATURE (1375)..(1379): This region may encompass “SGGQQ,” “GAGQQ,” “GQGPY,” “AGQQ” or “SQ,” wherein some positions may be absent
  MISC_FEATURE (1383)..(1387): This region may encompass “SGGQQ,” “GAGQQ,” “GQGPY,” “AGQQ” or “SQ,” wherein some positions may be absent
  MISC_FEATURE (1391)..(1395): This region may encompass “SGGQQ,” “GAGQQ,” “GQGPY,” “AGQQ” or “SQ,” wherein some positions may be absent
  MISC_FEATURE (1399)..(1403): This region may encompass “SGGQQ,” “GAGQQ,” “GQGPY,” “AGQQ” or “SQ,” wherein some positions may be absent
  MISC_FEATURE (1407)..(1411): This region may encompass “SGGQQ,” “GAGQQ,” “GQGPY,” “AGQQ” or “SQ,” wherein some positions may be absent
  MISC_FEATURE (1415)..(1419): This region may encompass “SGGQQ,” “GAGQQ,” “GQGPY,” “AGQQ” or “SQ,” wherein some positions may be absent
  MISC_FEATURE (1423)..(1427): This region may encompass “SGGQQ,” “GAGQQ,” “GQGPY,” “AGQQ” or “SQ,” wherein some positions may be absent
  MISC_FEATURE (1364)..(1427): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1431)..(1440): This region may encompass 6-10 residues
  MISC_FEATURE (1447)..(1451): This region may encompass “SGGQQ,” “GAGQQ,” “GQGPY,” “AGQQ” or “SQ,” wherein some positions may be absent
  MISC_FEATURE (1455)..(1459): This region may encompass “SGGQQ,” “GAGQQ,” “GQGPY,” “AGQQ” or “SQ,” wherein some positions may be absent
  MISC_FEATURE (1463)..(1467): This region may encompass “SGGQQ,” “GAGQQ,” “GQGPY,” “AGQQ” or “SQ,” wherein some positions may be absent
  MISC_FEATURE (1471)..(1475): This region may encompass “SGGQQ,” “GAGQQ,” “GQGPY,” “AGQQ” or “SQ,” wherein some positions may be absent
  MISC_FEATURE (1479)..(1483): This region may encompass “SGGQQ,” “GAGQQ,” “GQGPY,” “AGQQ” or “SQ,” wherein some positions may be absent
  MISC_FEATURE (1487)..(1491): This region may encompass “SGGQQ,” “GAGQQ,” “GQGPY,” “AGQQ” or “SQ,” wherein some positions may be absent
  MISC_FEATURE (1495)..(1499): This region may encompass “SGGQQ,” “GAGQQ,” “GQGPY,” “AGQQ” or “SQ,” wherein some positions may be absent
  MISC_FEATURE (1503)..(1507): This region may encompass “SGGQQ,” “GAGQQ,” “GQGPY,” “AGQQ” or “SQ,” wherein some positions may be absent
  MISC_FEATURE (1444)..(1507): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1511)..(1520): This region may encompass 6-10 residues
  MISC_FEATURE (1527)..(1531): This region may encompass “SGGQQ,” “GAGQQ,” “GQGPY,” “AGQQ” or “SQ,” wherein some positions may be absent
  MISC_FEATURE (1535)..(1539): This region may encompass “SGGQQ,” “GAGQQ,” “GQGPY,” “AGQQ” or “SQ,” wherein some positions may be absent
  MISC_FEATURE (1543)..(1547): This region may encompass “SGGQQ,” “GAGQQ,” “GQGPY,” “AGQQ” or “SQ,” wherein some positions may be absent
  MISC_FEATURE (1551)..(1555): This region may encompass “SGGQQ,” “GAGQQ,” “GQGPY,” “AGQQ” or “SQ,” wherein some positions may be absent
  MISC_FEATURE (1559)..(1563): This region may encompass “SGGQQ,” “GAGQQ,” “GQGPY,” “AGQQ” or “SQ,” wherein some positions may be absent
  MISC_FEATURE (1567)..(1571): This region may encompass “SGGQQ,” “GAGQQ,” “GQGPY,” “AGQQ” or “SQ,” wherein some positions may be absent
  MISC_FEATURE (1575)..(1579): This region may encompass “SGGQQ,” “GAGQQ,” “GQGPY,” “AGQQ” or “SQ,” wherein some positions may be absent
  MISC_FEATURE (1583)..(1587): This region may encompass “SGGQQ,” “GAGQQ,” “GQGPY,” “AGQQ” or “SQ,” wherein some positions may be absent
  MISC_FEATURE (1524)..(1587): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1591)..(1600): This region may encompass 6-10 residues
  MISC_FEATURE (1)..(1600): This sequence may encompass 2-20 “GGY-[GPG-X1]n1-GPS-(A)n2” repeating units, wherein X1 is “SGGQQ,” “GAGQQ,” “GQGPY,” “AGQQ” or “SQ,” n1 is 4-8 and n2 is 6-10 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAA

SEQ ID NO: 4 - protein, 5 aa, Artificial Sequence
  Description of Artificial Sequence Synthetic peptide
SGGQQ

SEQ ID NO: 5 - protein, 5 aa, Artificial Sequence
  Description of Artificial Sequence Synthetic peptide
GAGQQ

SEQ ID NO: 6 - protein, 5 aa, Artificial Sequence
  Description of Artificial Sequence Synthetic peptide
GQGPY

SEQ ID NO: 7 - protein, 4 aa, Artificial Sequence
  Description of Artificial Sequence Synthetic peptide
AGQQ

SEQ ID NO: 8 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 9 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 10 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 11 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 12 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 13 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 14 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 15 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 16 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 17 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 18 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 19 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 20 - protein, 32 aa, Euprosthenops australis
GGQGGQGQGRYGQGAGSSAAAAAAAAAAAAAA

SEQ ID NO: 21 - protein, 42 aa, Tetragnatha kauaiensis
GGLGGGQGAGQGGQQGAGQGGYGSGLGGAGQGASAAAAAAAA

SEQ ID NO: 22 - protein, 42 aa, Argiope aurantia
GGYGPGAGQQGPGSQGPGSGGQQGPGGLGPYGPSAAAAAAAA

SEQ ID NO: 23 - protein, 46 aa, Deinopis spinosa
GPGGYGGPGQQGPGQGQYGPGTGQQGQGPSGQQGPAGAAAAAAAAA

SEQ ID NO: 24 - protein, 42 aa, Nephila clavata
GPGGYGLGQQGPGQQGPGQQGPAGYGPSGLSGPGGAAAAAAA

SEQ ID NO: 25 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 26 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 27 - protein, 161 aa, Nephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 28 - protein, 186 aa, Nephilengys cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 29 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 30 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 31 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 32 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 33 - protein, 177 aa, Nephilengys cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 34 - protein, 174 aa, Nephilengys cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 35 - protein, 5 aa, Unknown
  Description of Unknown Silk polypeptide block sequence
SGAGG

SEQ ID NO: 36 - protein, 5 aa, Unknown
  Description of Unknown Silk polypeptide block sequence
GSGAG

SEQ ID NO: 37 - protein, 5 aa, Unknown
  Description of Unknown Silk polypeptide block sequence
GGSGA

SEQ ID NO: 38 - protein, 8 aa, Artificial Sequence
  Description of Artificial Sequence Synthetic peptide
  MISC_FEATURE (1)..(8): This sequence may encompass 6-8 residues
HHHHHHHH